(19) United States
(12) Patent Application Publication
(10) Pub. No.: US 2004/0152105 A1
(43) Pub. Date: Aug. 5, 2004

(54) Immune modulatory compounds and methods

(75) Inventors: Lorenz Vogt, Wetzikon (CH); Martin Bachmann, Seuzach (CH)

Correspondence Address: STERNE, KESSLER, GOLDSTEIN & FOX PLLC, 1100 NEW YORK AVENUE, N.W., WASHINGTON, DC, 20005, US

(73) Assignee: Cytos Biotechnology AG.

(21) Appl. No.: 10/656,269

(22) Filed: Sep. 8, 2003

Related U.S. Application Data
(60) Provisional application No. 60/449,583, filed on Feb. 26, 2003.
(60) Provisional application No. 60/408,233, filed on Sep. 6, 2002.

(51) Int. Cl. C12Q 1/68; C07H 21/04; C07K 14/74

(52) U.S. Cl. 435/6; 435/69.1; 435/320.1; 435/325; 530/350; 536/23.5

(57) ABSTRACT

The present invention relates to nucleic acids encoding novel polypeptides that modulate immune responses as well as corresponding recombinant vectors and host cells comprising said vectors. The invention also encompasses the above mentioned polypeptides, derivatives thereof, antibodies directed against said polypeptides and corresponding hybridoma cell lines. Furthermore, the invention is directed at pharmaceutical compositions comprising the above mentioned nucleic acids, vectors, polypeptides and/or antibodies. In addition, the present invention is directed to a method of identifying a compound that modulates a cell response, and a method of treating and/or preventing a disease in a mammal, wherein said disease benefits from an enhanced or reduced immune response. A further aspect provides a method of producing a polypeptide, nucleic acid, vector or antibody according to the invention.

### CROSS-REFERENCE TO RELATED APPLICATIONS

This application claims the benefit of U.S. Provisional Patent Application No. 60/449,583, filed Feb. 26, 2003, and U.S. Provisional Patent Application No. 60/408,233, filed Sep. 6, 2002, the contents of which are relied upon and incorporated by reference in their entireties.

### TECHNICAL FIELD OF THE INVENTION

The present invention relates to nucleic acids encoding novel polypeptides that modulate immune responses as well as corresponding recombinant vectors and host cells comprising said vectors. The invention also encompasses the above mentioned polypeptides, derivatives thereof, antibodies directed against said polypeptides and corresponding hybridoma cell lines. Furthermore, the invention is directed at pharmaceutical compositions comprising the above mentioned nucleic acids, vectors, polypeptides and/or antibodies. In addition, the present invention is directed to a method of identifying a compound that modulates a cell response, and a method of treating and/or preventing a disease in a mammal, wherein said disease benefits from an enhanced or reduced immune response. A further aspect provides a method of producing a polypeptide, nucleic acid, vector or antibody according to the invention

### BACKGROUND OF THE INVENTION

T cell lymphocytes (T cells) and B cell lymphocytes (B cells) are the primary cells of the specific immune system. Both are involved in acquired immunity and the complex interaction of these cell types is required for the expression of the full range of immune responses. T cells are specific for foreign antigens and the number of specific T cells must increase enormously in response for specific host defense.

The T cell response depends on two discrete receptor-ligand recognition events. The major event is the interaction of T cell receptors (TCRs) on the surface of the T cells with peptide-major histocompatibility complexes (pMHC) that are displayed on the surface of the antigen-presenting cell (APC) such as macrophages and dendritic cells. However, in the absence of a further costimulatory signal, the TCR-pMHC interaction alone is insufficient for producing complete T cell activation and may result in either apoptotic death or prolonged unresponsiveness of the responding T cell (Lenschow D. J. et al., (1996) Immunity 5, 285-93).

It is the interaction of a family of related costimulatory receptors with their respective ligands that furnishes the second costimulatory signals which are required for efficient T cell activation. Moreover, a second, complementary set of costimulatory receptors also provide negative signals that reduce the immune response and as such function to maintain the peripheral T cell tolerance to protect against autoimmunity (Nishimura H. et al., (1999) Immunity 11, 141-151; Nishimura H. et al., (2001) Science 291, 319-322; Greenwald R. J. et al., (2001) Immunity 14, 145-155).

Well known costimulatory ligands are the B7-1 (CD80) and B7-2 (CD86) molecules. Both belong to the immunoglobulin (Ig) superfamily, their extracellular regions being composed of a membrane distal Ig variable (IgV) domain and a membrane proximal Ig constant (IgC) domain. Said ligands bind CD28 and CTLA-4 that are expressed on T lymphocytes and are the best characterized costimulatory receptors (Linsley, P. S. et al., (1990) Proc. Natl. Acad. Sci. USA 87, 5031-5035; Linsley P. S. et al., (1991) J. Exp. Med. 174, 561-569).

CD28 is constitutively expressed on T cells and induces IL-2 secretion and T cell proliferation after binding by a costimulatory ligand (June, C. H. et al. (1990) Immunol. Today 11, 211-216). CTLA-4 is homologous to CD28 and occurs on T cells following activation (Freemann G. J. et al. (1992) J. Immunol. 149, 3795-3801). CTLA-4 has a significantly higher affinity for B7-1 than CD28 has and appears to inhibit rather than enhance T cell responses.

The B7 independence of some antigen-induced T-cell responses indicates the presence of additional B7-like co-stimulators. A number of further B7-like molecules have been identified.

B7-H1 (B7 homolog 1) shares about 25% amino acid identity and a similar overall structure with B7-1 and B7-2 (Dong H. et al. (1999) Nature Med. 5, 1365-1369). B7-H1-Ig fusion protein costimulates T cell growth and enhances mixed lymphocyte responses to alloantigens. Interaction of B7-H1 with a putative receptor on T cells preferentially induces secretion of interleukin 10 (IL-10) and interferon γ (IFN-γ) in the presence of an antigenic signal. In vitro binding assay indicate that B7-H1 does not bind to the receptors CD28 or CTLA-4 or the inducible costimulator (ICOS) (Hutloff A. et al. (1999) Nature 397, 263-266). A recent study suggested that PD-1 (Ishida Y. et al. (1992) EMBO J. 11, 3887-3895), a CTLA-4-like molecule, is a receptor for B7-H1 (Freeman G. J. et al. (2000) J. Exp. Med. 192, 1027-1034).

Another B7-like molecule of mouse origin is B7h being induced by tumor necrosis factor α (TNF-α) (Swallow M. M. et al. (1999) Immunity 11, 423-432). A number of authors demonstrated that B7h is a ligand for mouse ICOS (Yoshinaga S. K. et al. (1999) Nature 402, 827-832; Ling V. et al. (2000) J. Immunol. 164, 1653-1657; Mages H. W. et al. (2000) Eur. J. Immunol. 30, 1040-1047; Brodie D. et al. (2000) Curr. Biol. 10, 333-336). The human ortholog of mouse B7h is also known as B7-H2 (Wang S. et al. (2000) Blood 96, 2808-2813), GL50 (Ling V. et al. (2000) J. Immunol. 164, 1653-1657) or B7RP-1 (Yoshinaga S. K. et al. (2000) Int. Immunol. 12, 1439-1447) and its costimulatory function for T cell growth and cytokine production was confirmed (Wang S. et al. (2000) Blood 96, 2808-2813). Blocking the interaction of ICOS and its ligand with an ICOS-Ig fusion protein inhibits dendritic cell (DC)-mediated allogeneic responses (Aicher A. et al. (2000) J. Immunol. 164, 4689-4696).

A further member of the B7 family is B7-H3, which was identified by bioinformatical analysis (Chapoval A. I. et al. (2001) Nature Immunol. 2, 269-274; WO 02/10187 A1). B7-H3 binds a putative counter-receptor on activated T cells that is distinct form CD28, CTLA-4, ICOS and PD-1. Interaction of B7-H3 and its T cell counter-receptor induces proliferation of both CD4+ and CD8+T cells and enhances the induction of cytotoxic T cells (CTLs). Additionally B7-H3-Ig fusion protein selectively increases production of IFN-γ.

Another member of the B7 superfamily recently described is B7-H4 (Sica G. L. et al. (2003) Immunity 18, 849-861; also known as B7S1 (Durbaka V. R. (2003) Immunity 18, 863-873; B7x (Watanabe N. (2003) Nat. Immunol. 7, 670-679) which has been described as being a negative regulator of T cell activation. The putative counter receptor is BTLA, an immunoglobulin domain-containing glycoprotein expressed during activation of T cell and on T helper cell.

Although CD28-B7-mediated costimulation is essential for the activation of naïve T cells, it is usually not required for memory and effector T cell responses (Schweitzer A. N. et al. (1998) J. Immunol. 161, 2762-2771), suggesting that more complex regulatory pathways exist that involve additional receptor-ligand interactions. This idea was supported by the identification of additional costimulatory receptor-ligand pairs, such as inducible costimulator (ICOS)-B7-H2 (Hutloff A. et al. (1999) Nature 397, 263-266; Swallow M. M. et al. (1999) Immunity 11, 423-432; Yoshinaga S. K. et al. (1999) Nature 402, 827-832) and PD-1-PD-L (Ishida Y. et al. (1992) EMBO J. 11, 3887-3895; Freeman G. J. et al. (2000) J. Exp. Med. 192, 1027-1034; Latchman Y. et al. (2001) Nature Immunol. 2, 261-268; Tseng S. Y. et al. (2001) J. Exp. Med. 193, 839-846). The interaction between ICOS, a CD28 and CTLA-4 homolog (24% and 17% identity, respectively), and B7-H2, a B7 homolog (about 20% sequence identity with B7-1 and B7-2), stimulates both CD4+ and CD8+T cell responses. In contrast to the positive signal that ICOS-B7-H2 interaction delivers to T cells, the engagement of PD-1 on T cells by its PD-L ligands present on APCs and other nonlymoid cells is responsible for the delivery of inhibitory signals to the responding T cell. These inhibitory signals are important for both, the maintenance of self-tolerance and the down-regulation of T cell activity at sites of immune activation. Using ICOS-deficient mice it was demonstrated that ICOS is required for humoral immune responses after immunization with several antigens (Dong C. et al. (2001) Nature 409, 97-101; Dong C. et al. (2001) J. Immunol. 166, 3659-3662). Moreover, ICOS-deficient mice show greatly enhanced susceptibility to experimental autoimmune encephalomyelitis, thus suggesting that ICOS plays a protective role in inflammatory autoimmune diseases. Thus, members of the B7 costimulator family are important regulators in the immune response.

B lymphocytes (also referred to as B cells) mature within the bone marrow and leave the marrow expressing a unique antigen-binding membrane receptor. The B-cell receptor is a membrane-bound immunoglobulin glycoprotein. When a B cell encounters the antigen for which its membrane-bound antibody is specific, the cell begins to divide very rapidly; its progeny differentiate into memory B cells and effector cells called plasma cells. Memory B cells have a longer lifespan and continue to express membrane-bound antibody with the same specificity as the original parent cell. Plasma cells do not produce membrane-bound antibody but instead produce the antibody in a form that can be secreted. In the adult mouse, T and B lymphocytes are produced continuously either in the primary lymphoid organs or by peripheral cell division, the total number of T and B cells however remains constant. The mechanisms that determine the number of peripheral lymphocytes are poorly understood, but it is likely that population sizes are conditioned by multiple influences. The ensemble of stimulatory or inhibitory cellular interactions, growth factors, antigen etc. that condition cell survival and/or cell growth are referred to as resources (Freitas A. A. et al. (1995) Eur. J. Immunol. 25, 1729-38), cells sharing common resources belonging to the same “niche”. The homeostatic control of cell numbers suggests that resources are present in limited amounts, and that lymphocyte populations must compete for survival signals (Freitas A. A. et al. (1995) Eur. J. Immunol. 25, 1729-38; Freitas A. A. et al. (1996) Eur. J. Immmunol. 26, 2640-49). Evaluation of cell populations in different lines of mutant mice indicates that B- and T-cell numbers are independently regulated. The number of mature B-cells is similar in normal mice of in mice which lack T cells (TcR ko) (Mombaerts P. et al. (1992) Nature 360, 225-231), and the number of T cells is similar in normal mice and in mice that lack B cells (μMT ko) (Kitamura D et al. (1991) Nature 350, 423-426). It is believed that survival of newly produced B cell is dertermined not only by the direct interactions between each B cell and its ligand, but is also conditioned by the presence of other B lymphocytes, that compete for limited resources (Agenes F. et al. (1997) Eur. J. Immunol. 27, 1801-07). In chimeras reconstituted with mixtures of bone marrow (BM) cells from nomal and B-cell deficient donors, the number of pre-B cells produced was strictly dependent on the size of the immature stem-cell compartment. Moreover, the per-cell rates of pre-B cell division and of B-cell production were constant and independent of the number of peripheral mature B cells, suggesting the absence of regulatory feedback loops between the central and the peripheral B-cell compartments (Agenes F. et al. (1997) Eur. J. Immunol. 27, 1801-07). The size of peripheral B-cell pool was not determined by the number of immediate precursor cells or the rate of B-cell production. Mice with diminished numbers of pre-B cells and reduced rate of bone marrow B-cell production could generate full sized peripheral B-cell compartment (Tanchot C. et al. (1997) Immunology 9, 331-337). In B-cell deficient chimeras generated by injecting variable ratios of BM cells from B-cell deficient μMT donors and competent BM cells from normal mice, it was found that the number of activated IgM-secreting B cells was constant and independent of the number of pre-B and mature B-cells (Agenes F. et al. (1997) Eur. J. Immunol. 27, 1801-07). These results indicate that the number of activated B cells is not a constant fraction of the number of resting B cells, but must represent an autonomous B-cell compartment with different homeostatic controls. The independent homeostatic regulation of the resting and activated B-cell compartements allow the immune system to favour as a first priority, the maintenance of normal serum IgM and IgG levels.

In summary, B cell and T cell responses depend on multiple and complex interdependent events. Because of its key role in immunity, B cell and T cell regulation is a major target for treating and/or preventing a large variety of diseases that require or benefit from an enhanced or reduced immunity, e.g. autoimmune diseases including type I diabetes and multiple sclerosis, asthma, arthritis, myasthenia gravis, lupus erythematosus, pemhigus, psoriasis, colitis or rejection of transplanted organs, such as xenotransplants, immuno deficiency diseases, and cancer. Therefore, there is a strong need for compounds capable of modulating the complex B cell and T-cell responses for the purpose of treating and preventing numerous disorders in mammals. The present invention provides new compounds and methods for such a medical treatment. This and other objects of the present invention, as well as additional inventive features, will be apparent from the detailed description provided herein.

### SUMMARY OF THE INVENTION

The present invention provides isolated, and preferably purified, nucleic acids encoding polypeptides that modulate immune responses. Moreover, the present invention relates to nucleic acid operably linked to a promoter, recombinant vectors comprising said nucleic acids, and host cell comprising said vectors.

The invention also encompasses polpeptides encoded by said nucleic acids and functional derivatives thereof, antibodies directed against said polypeptides and hybridoma cell lines for producing said antibodies. The invention further encompasses cell lines transfected to express said antibodies.

Furthermore, the invention is directed at pharmaceutical compositions comprising the above mentioned nucleic acids, vectors, polypeptides and/or antibodies.

In addition, one aspect of the invention is directed at the above mentioned nucleic acids, vectors, peptides and/or antibodies for use as a medicament as well as for the preparation of a medicament for modulating the immune system, preferably for treating and/or preventing autoimmune diseases including type I diabetes and multiple sclerosis, asthma, arthritis, psoriasis, myasthenia gravis, lupus erythematosus, pemhigus, colitis or rejection of transplanted organs such as xenotransplants, immuno deficiency diseases, and cancer.

Another aspect of the present invention is directed at a method for identifying a compound that modulates an immune response, which method comprises: (i) contacting either B cells and/or T cells with a polypeptide according to the invention in the absence or presence of a compound of interest; and (ii) comparing the B cell and/or T cell response in the absence of said compound of interest with the B cell and/or T cell response in the presence of said compound of interest.

Still further provided by the present invention is a method of treating and/or preventing a disease in a mammal, wherein said disease is selected from autoimmune diseases and diseases that benefit from an enhanced or reduced immune response, preferably type I diabetes and multiple sclerosis, asthma, arthritis, myasthenia gravis, lupus erythematosus, pemhigus, psoriasis, colitis or rejection of transplanted organs such as xenotransplants, immuno deficiency diseases, and cancer, which method comprises administering to the mammal a therapeutically effective amount of a nucleotide, vector, polypeptide or antibody according to the invention. Furthermore, since the present invention is also preferably related to modulation of antibody and B cell responses in vivo, a method of treating and/or preventing a disease in a mammal is provided, wherein said disease is selected from autoimmune diseases mediated by antibodies including, preferably consisting of, myasthenia gravis, lupus erythematosus, pemhigus, and rejection of xenotransplants, which method comprises administering to the mammal a therapeutically effective amount of a nucleotide, vector, polypeptide or antibody according to the invention. Moreover, since the present invention is also preferably related to modulation of T cell responses in vivo, a method of treating and/or preventing a disease in a mammal is provided, wherein said disease is selected from autoimmune diseases including, and preferably consisting of, type I diabetes and multiple sclerosis, asthma, arthritis, psoriasis, colitis or rejection of transplanted organs such as xenotransplants, immuno deficiency diseases, and cancer, which method comprises administering to the mammal a therapeutically effective amount of a nucleotide, vector, polypeptide or antibody according to the invention.

In view of the foregoing, the present invention also provides a method of producing a polypeptide according to the invention, wherein a host cell of the present invention is cultured to produce said polypeptides.

Similarly provided is a method of producing an antibody according to the present invention, wherein a hybridoma cell line of the present invention is cultured to produce said antibodies or wherein a cell line transfected to express said antibodies is cultured.

### BRIEF DESCRIPTION OF THE FIGURES AND SEQUENCES

FIG. 1A is a line graph showing the proliferative response of purified murine B cells activated by different concentration of mB7-H5-Fc fusion protein in the absence or presence of different concentration of goat anti-mouse IgM antibody (coated onto tissue culture well plates).

FIG. 1B is a line graph showing the proliferative response of purified murine B cells activated by different concentration of mouse γ-globuline in the absence or presence of different concentration of goat anti-mouse IgM antibody (coated onto tissue culture well plates).

FIG. 2A is a bar graph showing the negative regulation of the proliferative response of purified murine CD4+ and CD8+T cells activated by anti-CD3 monoclonal antibody (coated onto tissue culture well bottoms using concentration of 0.5 μg/ml) and co-coated by either control mouse γ-globuline, mB7-H6-Fc fusion protein, or mB7-H5-Fc fusion protein. Proliferation was measured after 72 hours. Thesa data are representative of more than three independent experiments.

FIG. 2B is a bar graph showing the negative regulation of the proliferative response of purified murine CD4+ and CD8+T cells activated by 0.5 μg/ml anti-CD3 monoclonal antibody, different concentration of anti-CD28 monoclonal antibody and of mB7-H6-Fc fusion protein, mPD-L1-Fc fusion protein, or mPD-L2-Fc fusion protein, each coated onto tissue culture well bottoms using a concentration of 5 μg/ml. As control mouse γ-globuline was used. Proliferation was measured after 72 hours.

FIG. 3A depicts the disequilibrated homeostatic control of the isotype switched B cells following mB7-H5-Fc fusion protein administration. The bar graph shows the percentage of isotype switched B cells of CD19 positive cells. The experimental groups, that obtained mB7-H5-Fc fusion protein showed a fivefold upregulation compared to the control group.

FIG. 3B depicts the disequilibrated homeostatic control of the lymphocytes following mB7-H5-Fc fusion protein administration. The bar graph shows the percentage of the following groups, isotype switched B cells, naïve mature B cells and T cell, macrophages, granulocytes and the rest. The analysis was performed by staining of lymphocyte surface markers and FACS.

FIG. 4A depicts the disequilibrated homeostatic control of the lymphocytes following mB7-H5-Fc fusion protein administration. The bar graph shows the percentage of the following groups, isotype switched B cells, naïve mature B cells and T cell, macrophages, granulocytes and the rest. The analysis was performed by staining of lymphocyte surface markers and FACS.

FIG. 4B depicts the downregulation of the Qβ specific B cells evoked by the administration of mB7-H5-Fc fusion protein in vivo. The bar graph shows the percentage of the Qβ specific B cells of isotype switched B cells for the different experimental groups.

FIG. 5A depicts the downregulation of the Qβ specific isotype switched B cells evoked by the administration of mB7-H6-Fc fusion protein. The bar graph shows the percentage of the Qβ specific B cells of isotype switched B cells for the different experimental groups.

FIG. 5B depicts the downregulation of the number of Qβ specific antibody forming cells (AFC) evoked by the administration of mB7-H6-Fc fusion protein. The bar graph shows the numbers of Qβ specific AFC per 10⁶ splenocytes.

### DETAILED DESCRIPTION OF THE INVENTION

1. Definitions

Animal: As used herein, the term “animal” is meant to include, for example, humans, sheep, elks, deer, mule deer, minks, mammals, monkeys, horses, cattle, pigs, goats, dogs, cats, rats, mice, birds, chicken, reptiles, fish, insects and arachnids.

Antibody: As used herein, the term “antibody” refers to molecules which are capable of binding an epitope or antigenic determinant. The term is meant to include whole antibodies and antigen-binding fragments thereof, including single-chain antibodies. Most preferably the antibodies are human antigen binding antibody fragments and include, but are not limited to, Fab, Fab′ and F(ab′)₂, Fd, single-chain Fvs (scFv), single-chain antibodies, disulfide-linked Fvs (sdFv) and fragments comprising either a V_{L} or V_{H} domain. The antibodies can be from any animal origin including birds and mammals. Preferably, the antibodies are human, murine, rabbit, goat, guinea pig, camel, horse or chicken. As used herein, “human” antibodies include antibodies having the amino acid sequence of a human immunoglobulin and include antibodies isolated from human immunoglobulin libraries or from animals transgenic for one or more human immunoglobulins and that do not express endogenous immunoglobulins, as described, for example, in U.S. Pat. No. 5,939,598 by Kucherlapati et al. The term “antibody” may futher include humanized antibodies wherein the antigen-binding parts of the humanized antibody are derived from a non-human species and the remaining parts of the humanized antibody display a human amino acid sequence.

Derivative: The term “derivative”, as used herein, means that the amino acid sequence of any of the polypeptides encompassed by the present invention is preferably at least 50%, more preferably at least 80%, and even more preferably at least 90%, and most preferably at least 95% identical to the polypeptide sequence encoded by any of the nucleic acids according to the invention, preferably at least 50%, more preferably at least 80%, and even more preferably at least 90%, and most preferably at least 95% identical to the polypeptide sequence of hsB7-H4LV (SEQ ID NO:2), hsB7-H4LV(ECD) (SEQ ID NO:4), hsB7-H5 (SEQ ID NO:6), hsB7-H5(ECD) (SEQ ID NO:8), mB7-H5 (SEQ ID NO:10), mB7-H5(ECD) (SEQ ID NO: 12), mB7-H6 (SEQ ID NO:14), mB7-H6(ECD) (SEQ ID NO: 16), hsB7-H6 (SEQ ID NO: 42), or hsB7-H6(ECD) (SEQ ID NO: 44). ECD means extracellular domain of the polypeptides of the invention.

The term “functional derivative” refers to polypeptide derivatives that are fully functional in comparison to any of the polypeptide sequences (i) hsB7-H4LV (SEQ ID NO:2), (ii) hsB7-H4LV(ECD) (SEQ ID NO:4), (iii) hsB7-H5 (SEQ ID NO:6), (iv) hsB7-H5(ECD) (SEQ ID NO:8), (v) mB7-H5 (SEQ ID NO:10), (vi) mB7-H5(ECD) (SEQ ID NO: 12), (vii) mB7-H6 (SEQ ID NO: 14), (viii) mB7-H6(ECD) (SEQ ID NO: 16), (ix) hsB7-H6 (SEQ ID NO: 42), or (x) hsB7-H6(ECD) (SEQ ID NO: 44) or which retain at least some, preferably at least 20%, more preferably at least 50%, and most preferably at least 90% of the biological activity of any of (i) to (x). Moreover, the term functional derivative preferably encompasses a functional fragment, variant (e.g., structurally and functionally similar to any of the proteins of (i) to (x) and has at least one functionally equivalent domain), analog (e.g., a protein or fragment thereof substantially similar in function to any one of the proteins of (i) to (x) or fragment thereof), chemical derivative (e.g., contains additional chemical moieties, such as polyethyleneglycol and derivatives thereof), or peptidomimetic (e.g., a low molecular weight compound that mimics a polypeptide in structure and/or function (see, e.g., Abell, Advances in Amino Acid Mimetics and Peptidomimetics, London: JAI Press (1997); Gante, Peptidmimetica—massgeschneiderte Enzyminhibitoren Angew. Chem. 106: 1780-1802 (1994); and Olson et al., J. Med. Chem. 36: 3039-3049 (1993) ) of any of the above mentioned polypeptides (i), (ii), (iii), (iv), (v), (vi), (vii), (viii), (ix) or (x). In a further preferred embodiment of the present invention, said functional derivative of (i), (ii), (iii), (iv), (v), (vi), (vii), (viii), (ix) or (x) is a fusion molecule or fusion protein thereof. It is understood that polypeptides, fusion proteins, fusion molecules and protein complexes coupled with the polypeptides or functional polypeptide derivatives are also preferably encompassed by the term “functional polypeptide derivative”. Preferably, a functional polypeptide of the invention or a derivative thereof is capable of modulating an immune response, preferably B cell and/or T cell activation.

Effective Amount: As used herein, the term “effective amount” refers to an amount necessary or sufficient to realize a desired biologic effect. An effective amount of the composition would be the amount that achieves this selected result, and such an amount could be determined as a matter of routine by a person skilled in the art. For example, an effective amount for treating an immune system deficiency could be that amount necessary to cause activation of the immune system, resulting in the development of an antigen specific immune response upon exposure to antigen. The term is also synonymous with “sufficient amount.”

The effective amount for any particular application can vary depending on such factors as the disease or condition being treated, the particular composition being administered, the size of the subject, and/or the severity of the disease or condition. One of ordinary skill in the art can empirically determine the effective amount of a particular composition of the present invention without necessitating undue experimentation.

Functional: The term “functional”, as used herein, relates to the ability of the nucleic acids and/or polypeptides of the invention to modulate immune response, in particular T cell and B cell response. “Non-functional polypeptides do not modulate T or B cell response but may also be useful, e.g. in that they may be used to produce antibodies that bind functional and/or non-functional polypeptides according to the invention.

Fusion: As used herein, the term “fusion” refers to the combination of amino acid sequences of different origin in one polypeptide chain by in-frame combination of their coding nucleotide sequences. The term “fusion” explicitly encompasses internal fusions, i.e., insertion of sequences of different origin within a polypeptide chain, in addition to fusion to one of its termini.

Isolated and purified nucleic acid: The term “isolated and purified nucleic acid” as used herein means a nucleic acid free of the genes that flank the gene of interest in the genome of an organism in which the gene of interest naturally occurs. The term therefore includes a recombinant nucleic acid incorporated into a vector, into an autonomously replicating plasmid or virus, or into the genomic nucleic acid sequence of a prokaryote or eukaryote. It also includes a separate nucleic acid molecule such as a cDNA; a genomic fragment; a fragment produced by polymerase chain reaction (PCR); a restriction fragment; a DNA, RNA, or PNA encoding a non-naturally occurring protein, fusion protein, or fragment of a given protein; or a nucleic acid which is a degenerate variant of a naturally occurring nucleic acid. In addition, it includes a recombinant nucleotide sequence that is part of a hybrid gene, i.e. a gene encoding a fusion protein. Also included is a recombinant nucleic acid that encodes a polypeptide according to SEQ ID NOs: 2, 6, 10, 14, 42 or a functional derivative thereof, or that encodes the extracellular domain according to SEQ ID NOs: 4, 8, 12, 16, 44 or a functional derivative thereof. From the above it is clear that an isolated and purified nucleic acid does not include a restriction fragment containing all or part of a gene that flanks the gene of interest in the genome of the organism in which the gene of interest naturally occurs. Furthermore, an isolated and purified nucleic acid does not mean a nucleic acid present among hundreds to millions of other nucleic acid molecules within, for example, total cDNA or genomic libraries or genomic DNA or RNA restriction digests in, for example, a restriction digest reaction mixture or an electrophoretic gel slice.

Immune response: As used herein, the term “immune response” refers to a humoral immune response and/or cellular immune response leading to the activation or proliferation of B- (B cell response) and/or T-lymphocytes (T cell response), dendritic cells, macrophages, and/or and antigen presenting cells. “Immunogenic” refers to an agent used to stimulate the immune system of a living organism, so that one or more functions of the immune system are increased and directed towards the immunogenic agent. An “immunogenic polypeptide” is a polypeptide that elicits a cellular and/or humoral immune response, whether alone or linked to a carrier in the presence or absence of an adjuvant. Preferably, antigen presenting cell may be activated.

A substance which “modulates” an immune response refers to a substance in which an immune response is observed that is enhanced, greater or intensified or reduced or weakened or deviated in any way with the addition of the substance when compared to the same immune response measured without the addition of the substance. For example, the lytic activity of cytotoxic T cells can be measured, e.g. using a 51Cr release assay, in samples obtained with and without the use of the substance during immunization. The amount of the substance at which the CTL lytic activity is enhanced as compared to the CTL lytic activity without the substance is said to be an amount sufficient to enhance the immune response of the animal to the antigen. In a preferred embodiment, the immune response is enhanced or reduced by a factor of at least about 2, more preferably by a factor of about 3 or more. The amount or type of cytokines secreted may also be altered. Alternatively, the amount of antibodies induced or their subclasses may be altered.

Nucleic acid: As used herein, the term “nucleic acid” refers to an isolated, and preferably purified, nucleic acid, wherein said nucleic acid is selected from the group consisting of: (i) a nucleic acid comprising at least one of the nucleic acid sequences listed in SEQ ID NOs 1, 3, 5, 7, 9, 11, 13, 15, 41, and 43; (ii) a nucleic acid having a sequence of at least 80% identity, preferably at least 90% identity, more preferred at least 95% identity, most preferred at least 98% identity with any of the nucleic acid sequences listed in SEQ ID NOs 1, 3, 5, 7, 9, 11, 13, 15, 41, or 43; (iii) a nucleic acid that hybridizes to a nucleic acid of (i) or (ii); (iv) a nucleic acid, wherein said nucleic acid is derivable by substitution, addition and/or deletion of, preferably at least one nucleotide, more preferably up to 50 nucleotides, and even more preferably up to 100 nucleotides of, one of the nucleic acids of (i), (ii) or (iii); and (v) a fragment of any of the nucleic acids of (i), (ii), (iii), or (iv), that hybridizes to a nucleic acid of (i).

Hybridization: The term “nucleic acid” or “fragment of a nucleic acid that hybridizes” with one of the other nucleic acids, for example with one of the nucleic acids having a sequence of SEQ ID NOs 1, 3, 5, 7, 9, 11, 13, 15, 41, or 43 or any of the nucleic acids of the invention, indicates a nucleic acid sequence that hybridizes under stringent conditions with a counterpart of a nucleic acid having the features described hereinabove in (i) to (v). For example, hybridizing may be performed at 68° C. in 2×SSC or according to the protocol of the dioxygenine-labeling-kits of the Boehringer (Mannheim) company. A further example of stringent hybridizing conditions is, for example, the incubation at 65° C. overnight in 7% SDS, 1% BSA, 1 mM EDTA, 250 mM sodium phosphate buffer (pH 7.2) and subsequent washing at 65° C. with 2×SSC; 0.1% SDS.

Percent identity: The term “percent identity” as known to the skilled artisan and used herein indicates the degree of relatedness among 2 or more nucleic acid molecules that is determined by agreement among the sequences. The percentage of “identity” is the result of the percentage of identical regions in 2 or more sequences while taking into consideration the gaps and other sequence peculiarities.

The identity of related nucleic acid molecules can be determined with the assistance of known methods. In general, special computer programs are employed that use algorithms adapted to accomodate the specific needs of this task. Preferred methods for determining identity begin with the generation of the largest degree of identity among the sequences to be compared. Computer programs for determining the identity among two sequences comprise, but are not limited to, the GCG-program package, including GAP (Devereux et al., Nucleic Acids Research 12 (12):387 (1984); Genetics Computer Group University of Wisconsin, Madison, (WI)); BLASTP, BLASTN, and FASTA (Altschul et al., J. Molec. Biol 215:403/410 (1990) ). The BLAST X program can be obtained from the National Center for Biotechnology Information (NCBI) and from other sources (BLAST handbook, Altschul et al., NCB NLM NIH Bethesda, Md. 20894). Also, the well-known Smith-Waterman algorithm can be used for determining identity.

Preferred parameters for sequence comparison comprise the following:

| Algorithm | Needleman and Wunsch, J. Mol. Biol. |
|---|---|
| | 48: 443-453 (1970) |
| Comparison matrix | Matches = +10, mismatch 0 |
| Gap penalty: | 50 |
| Gap length penalty: | 3 |

The gap program is also suited to be used with the above-mentioned parameters. The above mentioned parameters are standard parameters (default) for nucleic acid comparisons.

Further exemplary algorithms, gap opening penalties, gap extension penalties, comparison matrix, including those in the program handbook, Wisconsin-package, version 9, September 1997, can also be used. The selection depends on the comparison to be done and further, whether a comparison among sequence pairs, for which GAP or Best Fit is preferred, or whether a comparison among a sequence and a large sequence databank, for which FASTA or BLAST is preferred, is desired.

Polypeptide: As used herein, the term “polypeptide” refers to a molecule composed of monomers (amino acids) linearly linked by amide bonds (also known as peptide bonds). It indicates a molecular chain of amino acids and does not refer to a specific length of the product. Thus, peptides, dipeptides, tripeptides, oligopeptides and proteins are included within the definition of polypeptide. This term is also intended to refer to post-expression modifications of the polypeptide, for example, glycosolations, acetylations, phosphorylations, and the like. A recombinant or derived polypeptide is not necessarily translated from a designated nucleic acid sequence. It may also be generated in any manner, including chemical synthesis.

The term “isolated and purified polypeptide” as used herein refers to a polypeptide or a peptide fragment which either has no naturally-occurring counterpart (e.g., a peptidomimetic), or has been separated or purified from components which naturally accompany it, e.g., in tissue such as pancreas, liver, lung, spleen, ovary, testis, muscle, joint tissue, neural tissue, gastrointestinal tissue, or body fluids such as blood, serum or urine. Preferably, a polypeptide is considered “isolated and purified” when it makes up for at least 60% (w/w) of a dry preparation, thus being free from most naturally-occurring polypeptides and/or organic molecules with which it is naturally associated. Preferably, a polypeptide of the invention makes up for at least 80%, more preferably at 90%, and most preferably at least 99% (w/w) of a dry preparation. Chemically synthesized polypeptides are by nature “isolated and purified” within the above context.

An isolated polypeptide of the invention may be obtained, for example, by extraction from a natural source (e.g., from human tissues or body fluids); by expression of a recombinant nucleic acid encoding the peptide; or by chemical synthesis. A polypeptide that is produced in a cellular system being different from the source from which it naturally originates is “isolated and purified”, because it is separated from components which naturally accompany it. The extent of isolation and/or purity can be measured by any appropriate method, e.g., column chromatography, polyacrylamide gel electrophoresis, HPLC analysis, NMR spectroscopy, gas liquid chromatography, or mass spectrometry. Preferably, polypeptides according to the invention are selected from the group consisting of: (i) hsB7-H4LV (SEQ ID NO:2); (ii) hsB7-H5 (SEQ ID NO:6); (iii) mB7-H5 (SEQ ID NO:10) (iv) mB7-H6 (SEQ ID NO:14); (v) hsB7-H6 (SEQ ID NO: 42) and (vi) a functional derivative of (i), (ii), (iii), (iv) or (v). Further preferred are the above mentioned polypeptides hsB7-H4LV, hsB7-H5, mB7-H5, mB7-H6 and hsB7-H6 that are derived by conservative substitutions. Conservative substitutions typically include substitutions within the following groups: glycine and alanine; valine, isoleucine, and leucine; aspartic acid and glutamic acid; asparagines, glutamine, serine and threonine; lysine histidine and arginine; and phenylalanine and tyrosine.

Immune response: As used herein, “the term immune response” includes T cell-mediated and/or B-cell mediated immune responses that are influenced by modulation of T cell costimulation. Exemplary immune responses include B cell responses (e.g., antibody production) T cell responses (e.g., cytokine production, and cellular cytotoxicity) and activation of cytokine responsive cells, e.g., macrophages.

Modulation: As used herein, the term “modulation” with respect to immune responses includes either down-modulation, i.e. meaning a reduction in any one or more immune responses and up-modulation, i.e. meaning an increase in any one or more immune responses. It will be understood that up-modulation of one type of immune response may lead to a corresponding down-modulation in another type of immune response.

T cell response: As used herein, the term “T cell response” refers to a cellular T cell response leading to the activation or proliferation of T-lymphocytes, e.g. a response by an increase in the number of T cells, by a change in the composition of molecules within or on the surface of T cells, by T cell migration, by a change in the lifespan of a T cell, or by a change of the quality and/or in the quantity of molecules released by T cells. T cells and T-lymphocytes, as used herein, are used interchangeably. Increased IgG responses are also reflecting enhanced T cell responses since IgG responses are dependent on the presence of T help cells.

A substance, e.g a polypeptide, a nucleic acid, or a vector of the invention, which “modulates” a T cell response refers to a substance in which a T cell response is observed that is greater or intensified or reduced or weakened or deviated in any way with the addition of the substance when compared to the same response measured without the addition of the substance. In addition, as used herein, a substance that modulates a T cell response is understood to indicate a substance that causes a T cell to respond to the contact of said substance to said T cell, e.g. respond by an increase in the number of T cells, by a change in the composition of molecules within or on the surface of T cells, or by a change of the quality and/or in the quantity of molecules released by T cells. Preferably, a substance, e.g. a polypeptide according to the invention, “co-stimulates” a T cell upon contacting a cell-surface molecule on a T cell, thereby enhancing a response of said T cell. A T cell response that results from a costimulatory interaction will be greater than said response in the absence of the substance. The response of the T cell in the absence of the co-stimulatory substance can be no response or it can be a response significantly lower than in the presence of the co-stimulatory substance. It is understood that the modulation of a T cell response incudes an effector, helper, or suppressive response. For example, the lytic activity of cytotoxic T cells can be measured, e.g. using a 51Cr release assay, in samples obtained with and without the use of the substance during immunization. The amount of the substance at which the CTL lytic activity is enhanced as compared to the CTL lytic activity without the substance is said to be an amount sufficient to enhance the immune response of the animal to the antigen. The amount or type of cytokines secreted may also be altered. Alternatively, the amount of antibodies induced or their subclasses may be altered.

Treatment: As used herein, the terms “treatment”, “treat”, “treated” or “treating” refer to prophylaxis and/or therapy. When used with respect to an infectious disease, for example, the term refers to a prophylactic or therapeutic treatment which increases the resistance of a subject to infection with a pathogen or, in other words, decreases the likelihood that the subject will become infected with the pathogen or will show signs of illness attributable to the infection, as well as a treatment after the subject has become infected in order to fight the infection, e.g., reduce or eliminate the infection or prevent it from becoming worse. When used with respect to an autoimmune disease, for example, the term refers to a prophylactic or therapeutic treatment which decreases the likelihood that the subject will develop an autoimmune disease or will show signs of illness attributable to the autoimmune disease, as well as a treatment after the subject has developed an autoimmune disease in order to fight the disease, e.g., enhance self-tolerance of the subject and prevent the immune system of the subject from mistakenly attacking and destroying own body-tissue. By “treating” is meant the slowing, interrupting, arresting or stopping of the progression of a disease or condition and does not necessarily require the complete elimination of all disease symptoms and signs. “Preventing” is intended to include the prophylaxis of a disease or condition, wherein “prophylaxis” is understood to be any degree of inhibition of the time of onset or severity of signs or symptoms of the disease or condition, including, but not limited to, the complete prevention of the disease or condition.

One, a, or an: When the terms “one,” “a,” or “an” are used in this disclosure, they mean “at least one” or “one or more,” unless otherwise indicated.

As will be clear to those skilled in the art, certain embodiments of the invention involve the use of recombinant nucleic acid technologies such as cloning, polymerase chain reaction, the purification of DNA and RNA, the expression of recombinant proteins in prokaryotic and eukaryotic cells, etc. Such methodologies are well known to those skilled in the art and can be conveniently found in published laboratory methods manuals (e.g., Sambrook, J. et al., eds., Molecular Cloning, A Laboratory Manual, 2nd edition, Cold Spring Harbor Laboratory Press, Cold Spring Harbor, N.Y. (1989); Ausubel, F. et al., eds., Current Protocols in Molecular Biology, John H. Wiley & Sons, Inc. (1997) ). Fundamental laboratory techniques for working with tissue culture cell lines (Celis, J., ed., Cell Biology, Academic Press, 2^{nd} edition, (1998) ) and antibody-based technologies (Harlow, E. and Lane, D., Antibodies: A Laboratory Manual, Cold Spring Harbor Laboratory, Cold Spring Harbor, N.Y. (1988); Deutscher, M. P., “Guide to Protein Purification,” Meth. Enzymol. 128, Academic Press San Diego (1990); Scopes, R. K., Protein Purification Principles and Practice, 3rd ed., Springer-Verlag, New York (1994) ) are also adequately described in the literature, all of which are incorporated herein by reference.

2. Compositions and Methods for Modulating Immune Response

The present invention is relates to, at least in part, on the surprising and unexpected finding of human and mouse nucleic acid molecules encoding novel polypeptides that modulate immune responses and on the functional characterization of the polypeptides encoded by said nucleic acids.

In view of this finding, the present invention provides an isolated, and preferably purified, nucleic acid, wherein said nucleic acid is selected from the group consisting of: (i) a nucleic acid comprising, or preferably consisting essentially of, or preferably consisting of, at least one of the nucleic acid sequences listed in SEQ ID NOs 1, 3, 5, 7, 9, 11, 13, 15, 41, and 43; (ii) a nucleic acid having a sequence of at least 80% identity, preferably at least 90% identity, more preferred at least 95% identity, most preferred at least 98% identity with any of the nucleic acid sequences listed in SEQ ID NOs 1, 3, 5, 7, 9, 11, 13, 15, 41, or 43; (iii) a nucleic acid that hybridizes to a nucleic acid of (i) or (ii); (iv) a nucleic acid, wherein said nucleic acid is derivable by substitution, addition and/or deletion of, preferably at least one nucleotide, more preferably up to 50 nucleotides, and even more preferably up to 100 nucleotides of, one of the nucleic acids of (i), (ii) or (iii); and (v) a fragment of any of the nucleic acids of (i), (ii), (iii), or (iv), that hybridizes to a nucleic acid of (i).

In a further embodiment, the invention provides an isolated, and preferably purified, polypeptide comprising, or preferably consisting essentially of, or preferably consisting of a polypeptide sequence encoded by a nucleic acid of the invention. The preferred polypeptide sequences encoded by the nucleic acids according to the invention are the hsB7-H4LV (SEQ ID NO:2), hsB7-H4LV(ECD) (SEQ ID NO:4), hsB7-H5 (SEQ ID NO:6), hsB7-H5(ECD) (SEQ ID NO:8), mB7-H5 (SEQ ID NO:10), mB7-H5(ECD) (SEQ ID NO:12), mB7-H6 (SEQ ID NO: 14), mB7-H6(ECD) (SEQ ID NO: 16), hsB7-H6 (SEQ ID NO: 42) and the hsB7-H6(ECD) (SEQ ID NO: 44). These polypeptides are encoded by separate genes. The hsB7-H4LV polypeptide, the hsB7-H5 and hsB7-H6 polypeptide are human paraloges, whereas the mB7-H5 and mB7-H6 polypeptide are the mouse ortholog of the human hsB7-H5 and hsB7-H6 polypeptide, respectively. In a preferred embodiment, the nucleic acid of the invention encodes a protein that is capable of modulating an immune response, preferably a B cell and/or T cell response.

Moreover, in a preferred embodiment, the nucleic acids of the present invention also code for functional and non-functional derivatives of the above mentioned polypeptides. Preferably, the nucleic acid of the invention is a DNA, a RNA or a PNA.

The nucleic acid molecules according to the invention may be prepared synthetically by methods well-known to the skilled person, but also may be isolated from suitable DNA libraries and other publicly-available sources of nucleic acids and subsequently may optionally be mutated. The preparation of such libraries or mutations is well-known to the person skilled in the art.

In a preferred embodiment, the nucleic acid molecules of the invention are cDNA, genomic DNA, synthetic DNA or RNA, either double-stranded or single-stranded (i.e., either a sense or an antisense strand). In certain embodiments at least some of the nucleotide residues of the nucleic acids (sense or antisense) may be made resistant to nuclease degradation and these can be selected from residues such as phophorothioates and/or methylphosphonates. The antisense nucleic acids as hereinbefore described can advantageously be used as pharmaceuticals, preferred pharmaceutical applications being for the manufacture of a medicament for the prophylaxis or treatment of autoimmune diseases including type I diabetes and multiple sclerosis, asthma, arthritis, psoriasis, colitis or rejection of transplanted organs, immuno deficiency diseases, and cancer. Since the present invention is also related to modulation of antibody and B cell responses in vivo, autoimmune diseases mediated by antibodies may be particular attractive targets for therapeutic intervention. Therefore, further preferred pharmaceutical applications being for the manufacture of a medicament for the prophylaxis or treatment of autoimmune diseases mediated by antibodies including myasthenia gravis, which is mediated by antibodies specific for acetylcholine receptor; arthritis typically induced by antibodies specific for collagen and other proteins; lupus erythematosus, being a lethal auto-immune disease, mediated by antibodies specific for DNA; pemhigus where antibodies specific for demsosomes cause blistering of the skin. In all of these disease-conditions, lowering specific antibody titers result in reduced disease. Thus, in particular, modulation of B cell homeostasis by application of soluble B7-H5 or B7-H5 fusion molecules or antibodies directed against B7-H5 is a very preferred embodiment of the invention to reduce disease. Additional antibody mediated diseases include rejection of xenotransplants and. Fragments of these molecules, which are encompassed within the scope of the invention, may be produced by, for example, the polymerase chain reaction (PCR) or generated by treatment with one or more restriction endonucleases. A ribonucleic acid (RNA) molecule may be produced by in vitro transcription.

In a preferred embodiment, a nucleic acid according to the present invention encodes a polypeptide that is capable of modulating an immune response, preferably a B cell and/or T cell response.

As used herein, a polypeptide that modulates an immune response, preferably a B cell and/or a T cell response is understood to indicate a polypeptide that causes a B cell and/or T cell to respond to the contact of said polypeptide to said B cell and/or T cell, e.g. respond by an increase in the number of B cell and/or T cells, by a change in the composition of molecules within or on the surface of B cell and/or T cells, or by a change of the quality and/or in the quantity of molecules released by B cell and/or T cells.

Preferably, a polypeptide according to the invention “co-stimulates” a B cell and/or T cell upon contacting a cell-surface molecule on a B cell and/or T cell, thereby enhancing a response of said B cell and/or T cell. A B cell and/or T cell response that results from a costimulatory interaction will be greater than said response in the absence of the polypeptide. The response of the B cell and/or T cell in the absence of the co-stimulatory polypeptide can be no response or it can be a response significantly lower than in the presence of the co-stimulatory polypeptide. It is understood that the modulation of a immune response incudes an effector, helper, or suppressive response.

Exemplary “co-stimulatory” ligands include B7-1, B7-2, B7-H1, B7-H2, B7-H3, hsB7-H4LV, hsB7-H5, mB7-H5, mB7-H6, hsB7-H6,4-1BB, OX40L, and herpes virus entry mediator (HVEM). “Co-stimulatory” compounds may provide an “activating stimulus” by, e.g. enhancing intracellularly an activating signal received by a T cell through the antigen specific T cell receptor (TCR). An activating stimulus can be sufficient to elicit a detectable response in a T cell. However, a T cell usually requires co-stimulation (e.g., by hsB7-H4LV or hsB7-H5 or mB7-H5 or mB7-H6 polypeptide) in order to respond detectably to the activating stimulus. Examples of activating stimuli include, without being limited to, antibodies that bind to the TCR or to a polypeptide of the CD3 complex that is physically associated with the TCR on the T cell surface, alloantigens, or an antigenic peptide bound to a MHC molecule. Similar co-stimulatory receptors exist in B cells and myeloid cells such as CD21 or FcγRI.

Exemplary “inhibitory” compounds for T cells include B7-1, B7-2, PD-L1, PD-L2, B7-H4, hsB7-H4LV, hsB7-H5, mB7-H5, mB7-H6, and hsB7-H6. “Inhibitory” compounds may provide and “inhibitory signal” by transmitting a signal via an inhibitory receptor (e.g., CTLA-4, PD-1, and/or BTLA) molecule on an immune cell. Such a signal antagonizes a signal via the TCR and can result, e.g., in inhibition of: second messenger generation; proliferation; or effector function in the immune cell, e.g. cellular cytotoxicity, or the failure of the immune cell to produce mediators (such as cytokines (e.g., IL-2) and/or mediators of allergic responses); or development of anergy. Similar inhibitory receptors exist in B cells, NK cells and myeloid cells. Such receptors include CD22, NK-inhibitory receptors, and FcγRIIB.

In a further aspect the present invention provides new polypeptides. Preferably, said polypeptides are encoded by a nucleic acid according to the invention.

Preferably, polypeptides according to the invention are selected from the group consisting of: (i) hsB7-H4LV (SEQ ID NO:2), (ii) hsB7-H4LV(ECD) (SEQ ID NO:4), (iii) hsB7-H5 (SEQ ID NO:6), (iv) hsB7-H5(ECD) (SEQ ID NO:8), (v) mB7-H5 (SEQ ID NO: 10), (vi) mB7-H5(ECD) (SEQ ID NO: 12), (vii) mB7-H6 (SEQ ID NO: 14), (viii) mB7-H6(ECD) (SEQ ID NO: 16), (ix) hsB7-H6 (SEQ ID NO: 42), (x) hsB7-H6(ECD) (SEQ ID NO: 44) and (xi) a functional derivative of (i), (ii), (iii), (iv), (v), (vi), (vii), (viii), (ix) or (x).

In a further preferred embodiment of the present invention, said functional derivative of (i), (ii), (iii), (iv), (v), (vi), (vii), (viii), (ix) or (x) is a fusion molecule or fusion protein thereof. Co-stimulatory ligands are usually membrane bound and activate their counter-receptors by cross-linking. Thus, recombinant monovalent forms of co-stimulatory ligands fail to productively engage their receptors and may function as antagonists. In contrast, multivalent fusion molecules of co-stimulatory ligands (such as e.g. Fc fusion molecules) are therefore usually capable of triggering the respective co-stimulatory receptors. Thus, multivalent fusion molecules of activatory co-stimulatory ligands enhance responses by lymphocytes while multivalent fusion molecules of inhibitory co-stimulatory ligands inhibit responses of lymphocytes.

Since B7-H6 was surprisingly found to be an inhibitory receptor, multivalent fusion molecules (as the Fc fusion molecule used here) of B7-H6 are ideal substances to inhibit T cell response. Such fusion molecules may be used as drugs for therapy of T cell mediated diseases, such as T cell-mediated autoimmunity, including, and preferably, multiple sclerosis, arthritis, colitis, inflammatory bowel disease, Crohn's disease, type I diabetes and psoriasis. Rejection of transplanted organs is another preferred disease preventable by such drugs. In addition, chronic inflammatory disases caused by infection or allergens, such as asthma, are preferred target diseases for such a drug. Recombinant monovalent forms of costimulatory ligands or monovalent fusion molecules antagonize the function of their natural, cell bound counterparts. Since B7-H6 naturally inhibits T cell responses, a monovalent form of B7-H6 or monovaltent fusion molecules will inhibit the inhibition thereby enhancing T cell responses. Treatment with monovalent forms of B7-H6 or monovalent fusion molecules may therefore effectively enhance T cell responses against cancer or during chronic viral infections. Application of monovalent forms of B7-H6 or monovalent fusion molecules may be particularly effective during periods of vaccination, in particular if co-delivered with the vaccine.

B7-H5 was surprisingly found to trigger proliferation of B cells and production of antibodies. Monovalent forms of B7-H5 or monovalent fusion molecules may therefore be useful for the treatment of autoimmune diseases caused by antibodies, including arthritis (arthritis may be caused by T cells, antibodies or both), Myasthenia gravis, pemphigus or lupus erythematosus. Rejection of xenotransplants is also caused in part by antibodies and treatment with monovalent forms of B7-H5 or monovalent fusion molecules may therefore inhibit this rejection. Diseases characterized by excessive proliferation of B cells, such as cancer caused by B cell lymphomas, in particular Hogkin-lymphoma, may also be treatable with monovalent forms of B7-H5 or monovalent fusion molecules.

Further preferred are the above mentioned polypeptides hsB7-H4LV, hsB7-H5, mB7-H5, mB7-H6 and hsB7-H6 that are derived by conservative substitutions. Conservative substitutions typically include substitutions within the following groups: glycine and alanine; valine, isoleucine, and leucine; aspartic acid and glutamic acid; asparagines, glutamine, serine and threonine; lysine histidine and arginine; and phenylalanine and tyrosine.

In a further preferred embodiment, the present invention is directed to a functional polypeptide or a derivative thereof that is capable of modulating an immune response, preferably a B cell and/or T-cell response, more preferably B cell and/or T cell activation.

In a further aspect, the present invention provides nucleic acids, wherein said isolated, and preferably purified, nucleic acid is operably linked to a promoter, preferably linked to a promoter selected from the group consisting of the MCK promoter, the RSV promoter, the CMV promoter, a tetracycline-regulatable promoter, a doxycycline-regulatable promoter, and a promoter capable of being recognized by RNA-dependent RNA polymerase. Said operably linked nucleic acids can be used for, e.g. vaccination.

Preferably, the isolated, and preferably purified, nucleic acid is in the form of a recombinant vector, preferably a viral vector. The selection of a suitable vector and expression control sequences as well as vector construction is within the ordinary skill in the art. Preferably, the viral vector is selected from the group consisting of an adenoviral vector, an adeno-associated viral vector, a retroviral vector, a Herpes simplex viral vector, a lentiviral vector, a Sindbis viral vector, or a Semliki forest viral vector. Preferably, the isolated, and preferably purified, nucleic acid encoding and expressing the protein or polypeptide is operably linked to a promoter selected from the group consisting of the MCK promoter, the CMV promoter, a tetracycline-regulatable promoter, and a doxycycline-regulatable promoter.

Suitable vectors are reviewed in Kay et al., Nature Medicine 7: 33-40 (2001); Somia et al., Nature Reviews 1: 91-99 (2000); and van Deutekom et al., Neuromuscular Disorders 8: 135-148 (1998). Preferably, the viral vector is an adenoviral vector (preferred examples are described in Acsadi et al., Hum. Gene Ther. 7(2): 129-140 (1996); Quantin et al., PNAS USA 89(7): 2581-2584 (1992); and Ragot et al., Nature 361 (6413): 647-650 (1993) ), an adeno-associated viral vector (preferred examples are described in Rabinowitz et al., Curr. Opin. Biotechnol. 9(5): 470-475 (1998) ), a retroviral vector (preferred examples are described in Federico, Curr. Opin. Biotechnol. 10(5): 448-453 (1999) ), a Herpes simplex viral vector (see, e.g., Latchman, Gene 264(1): 1-9 (2001) ), a lentiviral vector, a Sindbis viral vector, or a Semliki forest viral vector. Suitable promoters for operable linkage to the isolated and purified nucleic acid are known in the art. Preferably, the isolated and purified nucleic acid encoding the protein is operably linked to a promoter selected from the group consisting of the muscle creatine kinase (MCK) promoter (Jaynes et al., Mol. Cell Biol. 6: 2855-2864 (1986) ), the cytomegalovirus (CMV) promoter, a tetracycline-regulatable promoter (Gossen et al., PNAS USA 89: 5547-5551 (1992) ), and a doxycycline-regulatable promoter (Gossen et al. (1992), supra). Vector construction, including the operable linkage of a coding sequence with a promoter and other expression control sequences, is within the ordinary skill in the art.

The present invention provides recombinant expression vectors capable of replicating in a host cell, comprising one or more vector sequences and a nucleic acid sequence of the invention. In a preferred embodiment, said recombinant vector is capable of producing a polypeptide according to the invention. The construct for use as a pharmaceutical is also provided, as well as its use for the manufacture of a medicament for the prophylaxis or treatment of autoimmune diseases including, and preferably consisting of, type I diabetes and multiple sclerosis, asthma, arthritis, psoriasis, colitis or rejection of transplanted organs, immuno deficiency diseases, and cancer as well as, preferably, for the prophylaxis or treatment of autoimmune diseases mediated by antibodies including, and preferably consisting of, myasthenia gravis, arthritis, lupus erythematosus, pemhigus, and rejection of xenotransplants.

Therefore, in a further aspect of the present invention, a pharmaceutical composition is provided comprising a recombinant vector in accordance with the present invention and a pharmaceutically acceptable carrier.

An additional aspect of the present invention discloses host cells comprising a nucleic acid according to the invention, preferably transformed to produce polypeptides of the present invention. In a preferred embodiment, the host cell of the invention comprises the recombinant vector of the invention, said vector comprising a nucleic acid according to the invention and said vector being capable of producing a polypeptide of the invention. Preferred host cells are eukaryotic cells, more preferably insect cells or mammalian cells.

Another aspect of the present invention relates to antibodies that specifically bind any of the polypeptide according to the invention. Of particular interest are monoclonal antibodies that block the interaction of the polypeptides according to the intervention with their receptors. Alternatively, a mixture of monoclonal antibodies recognizing non-overlapping epitopes may be used. Such antibodies recognizing non-overlapping epitopes are able to simultaneously bind to the polypeptide according to the invention (i.e. there is no competition for binding). A person skilled in the art may therefore easily be able to identify such antibodies.

Preferably, said antibodies bind to the hsB7-H4LV, hsB7-H5, mB7-H5, mB7-H6, or hsB7-H6 polypeptides of SEQ ID NOs: 2, 6, 10, 14, and/or 42, even more preferably to the extracellular domain of these polypeptides, namely to the amino acid sequences of SEQ ID NOs: 4, 8, 12, 16, and/or 44.

The antibodies may be polyclonal or monoclonal antibody. As used herein, the term “antibody” refers not only to whole antibody molecules, but also to antigen-binding fragments, e.g., Fab, F(ab′)₂, Fv, and single chain Fv fragments. Also included are chimeric antibodies, preferably humanized antibodies.

It is understood that an antibody of the present invention that “binds specifically”to a polypeptide of the present invention does not bind substantially to B7-1, B7-2, B7-H1, B7-H2, B7-H3, PD-L2 or B7S1 (Durbaka V. R. et al. (2003) Immunity 18, 863-873).

In a preferred embodiment said antibody of the invention inhibits the capability of the polypeptides of the present invention to modulate immune responses, preferably B cell responses, T cell responses, or B cell and T cell responses. Co-stimulatory ligands regulate responses of lymphocytes by engaging costimulatory receptors on these lymphocytes. Monoclonal antibodies directs against costimulatory ligands therefore may inhibit the interaction of the costimulatory ligand with it's receptor and thereby antagonizes it's function. Since B7-H6 naturally inhibits T cell responses, a monoclonal antibody directed against B7-H6 will inhibit the inhibition thereby enhancing T cell responses. Treatment with monoclonal antibodies against B7-H6 may therefore effectively enhance T cell responses against cancer or during chronic viral infections. Application of monoclonal antibodies against B7-H6 may be particularly effective during periods of vaccination, in particular if co-delivered with the vaccine. B7-H5 was surprisingly found to trigger proliferation of B cells and production of antibodies. Monoclonal antibodies against B7-H5 and blocking the interaction of B7-H5 with it's receptor(s) may therefore be useful for the treatment of autoimmune diseases caused by antibodies, including arthritis (arthritis may be caused by T cells, antibodies or both), Myasthenia gravis, pemphigus or lupus erythematosus. Rejection of xenotransplants is also caused in part by antibodies and treatment with monoclonal antibodies against B7-H5 may therefore inhibit this rejection. Diseases characterized by excessive proliferation of B cells, such as cancer caused by B cell lymphomas, in particular Hogkin-lymphoma, may also be treatable with monoclonal antibdodies against B7-H5.

Monoclonal antibodies, more preferably humanized antibodies of the present invention are preferred. The preparation of monoclonal antibodies and humanization thereof is within the ordinary skill in the art. An antibody specific for the polypeptide of the invention can be easily obtained by immunizing an animal with an immunogenic amount of the polypeptide. Therefore, an antibody recognizing a particular polypeptide embraces both polyclonal antibodies and antisera which are obtained by immunizing an animal, and which can be confirmed to recognize the polypeptide of this invention by Western blotting, ELISA, immunostaining or other routine procedure known in the art.

It is well known that if a polyclonal antibody can be obtained by sensitization, a monoclonal antibody is secreted by the hybridoma, which may be obtained from the lymphocytes of the sensitized animal (Chapter 6, Antibodies A Laboratory Manual, Cold Spring Harbor Laboratory Press, 1988). Therefore, monoclonal antibodies recognizing the polypeptide of the invention are also provided. Methods of producing polyclonal and monoclonal antibodies are known to those of skill in the art and described in the scientific and patent literature, see, e.g., Coligan, Current Protocols in Immunology, Wiley/Green, NY (1991); Stites (eds.) Basic and Clinical Immunology (7th ed.) Lange Medical Publications, Los Altos, Calif., and references cited therein (Stites); Goding, Monoclonal Antibodies: Principles and Practice (2nd ed.) Academic Press, New York, N.Y. (1986); and Kohler (1975) Nature 256: 495. Such techniques include selection of antibodies from libraries of recombinant antibodies displayed in phage or similar on cells. See, Huse (1989) Science 246: 1275 and Ward (1989) Nature 341: 544. Recombinant antibodies can be expressed by transient or stable expression vectors in mammalian cells, as in Norderhaug (1997) J. Immunol. Methods 204: 77-87.

According to the invention, an “antibody” also embraces an active fragment thereof. An active fragment means a fragment of an antibody having activity of antigen-antibody reaction. Specifically named, these are active fragments, such as F(ab′)₂, Fab′, Fab, and Fv. For example, F(ab′)₂ results if the antibody of this invention is digested with pepsin, and Fab results if digested with papain. Fab′ results if F(ab′)₂ is reduced with a reagent such as 2-mercaptoethanol and alkylated with monoiodoacetic acid. Fv is a mono active fragment where the variable region of heavy chain and the variable region of light chain are connected with a linker. A chimeric antibody is obtained by conserving these active fragments and substituting the fragments of another animal for the fragments other than these active fragments. In particular, humanized antibodies are envisioned.

Thus, in the above respect, hybridoma cell lines expressing antibodies or cell lines transfected to express said antibodies that specifically bind a polypeptide of the invention present a further aspect. Preferably, hybridoma cell lines expressing monoclonal antibodies of the invention are provided.

An additional embodiment of the invention relates to the administration of a pharmaceutical or sterile composition, in conjunction with a pharmaceutically acceptable carrier. In a preferred embodiment such pharmaceutical compositions may consist of at least one of the following: (i) a functional polypeptide, a functional polypeptide derivative, a nucleic acid or recombinant vector encoding/expressing a functional polypeptide or a functional polypeptide derivative, an antibody of the present invention, or mimetics, agonists, antagonists or inhibitors of the functional polypeptide, all of the present invention, and (ii) a pharmaceutically acceptable carrier (or excipient).

In a further aspect of the present invention, a pharmaceutical composition comprising a nucleic acid according to the invention and a pharmaceutically acceptable carrier is provided. In another aspect, the present invention provides for a pharmaceutical composition a vector according to the invention and a pharmaceutically acceptable carrier. Moreover, in again a further aspect, the present invention provides a pharmaceutical composition comprising an antibody according to the invention and a pharmaceutically acceptable carrier.

Suitable carriers or excipients are well-known in the art. A carrier or excipient may be a solid, semi-solid or liquid material which may serve as a vehicle or medium for the active ingredient. One of ordinary skill in the art in the field of preparing compositions can readily select the proper form and mode of administration depending upon the particular characteristics of the product selected, the disease or condition to be treated, the stage of the disease or condition, and other relevant circumstances (Remington's Pharmaceutical Sciences, Mack Publishing Co. (1990) ). The proportion and nature of the pharmaceutically acceptable carrier or excipient are determined by the solubility and chemical properties of the pharmaceutically active compound being selected, the chosen route of administration, and standard pharmaceutical practice. The pharmaceutical preparation may be adapted for oral, parenteral or topical use and may be administered to the patient in the form of tablets, capsules, suppositories, solution, suspensions, or the like. The pharmaceutically active compounds of the present invention, while effective themselves, can be formulated and administered in the form of their pharmaceutically acceptable salts, such as acid addition salts or base addition salts, for purposes of stability, convenience of crystallization, increased solubility, and the like.

Another aspect of the present invention is directed at at least one of the following: a functional polypeptide, a functional polypeptide derivative, a nucleic acid or recombinant vector encoding/expressing a functional polypeptide or a functional polypeptide derivative, or an antibody according to the present invention for use as a medicament. Moreover, in another aspect, the present invention provides for a nucleic acid in accordance with the invention for use as a medicament. Furthermore, in again a further aspect, the present invention provides a recombinant vector in accordance with the present invention for use as a medicament.

With respect to the vectors of the present invention, to ensure effective transfer of the vectors of the present invention, it is preferred that about 1 to about 5,000 copies of the vector according to the invention be employed per cell to be contacted, based on an approximate number of cells to be contacted in view of the given route of administration, and it is even more preferred that about 3 to about 300 pfu enter each cell. However, this is merely a general guideline, which by no means precludes use of a higher or lower amount, as might be warranted in a particular application, either in vitro or in vivo. The actual dose and schedule can vary depending on whether the composition is administered in combination with other compositions, e.g., pharmaceutical compositions, or depending on interindividual differences in pharmacokinetics, drug disposition, and metabolism. Similarly, amounts can vary in in vitro applications depending on the particular type of cell or the means by which the vector is transferred. One skilled in the art easily can make any necessary adjustments in accordance with the necessities of the particular situation. Also in view of the above, the present invention provides an isolated and purified nucleic acid encoding the above-described protein or polypeptide, optionally in the form of a recombinant viral vector.

In a further aspect, the present invention encompasses the use of at least one of the following: a functional polypeptide, a functional polypeptide derivative, a nucleic acid or recombinant vector encoding/expressing a functional polypeptide or a functional polypeptide derivative, or an antibody according to the present invention for the preparation of a medicament for modulating the immune response. Moreover, in another aspect, the present invention provides for a nucleic acid in accordance with the invention for the preparation of a medicament for modulating the immune response. Furthermore, in again a further aspect, the present invention provides a recombinant vector in accordance with the present invention for the preparation of a medicament for modulating the immune response.

Preferably the above mentioned compounds, e.g. a functional polypeptide, a functional polypeptide derivative, a nucleic acid or recombinant vector encoding/expressing a functional polypeptide or a functional polypeptide derivative, or an antibody according to the present invention, a nucleic acid or a recombinant vector in accordance with the invention, are used for the preparation of a medicament for treating and/or preventing autoimmune diseases including, and preferably consisting of, type I diabetes and multiple sclerosis, asthma, arthritis, psoriasis, colitis or rejection of transplanted organs, immuno deficiency diseases, and cancer as well as, preferably, for the prophylaxis or treatment of autoimmune diseases mediated by antibodies including, and preferably consisting of, myasthenia gravis, arthritis, lupus erythematosus, pemhigus, and rejection of xenotransplants.

In a further preferred embodiment, the present invention relates to a method of identifying a compound that inhibits an immune response. The method involves (i) providing a test compound; (ii) culturing the compound, together with one or more functional polypeptides and/or functional polypeptide derivatives according to the invention, and a B cell or a T cell, or a B cell or a T cell activating stimulus together; and (iii) determining whether the test compound inhibits an immune response.

The invention also embodies a method of identifying a compound that enhances an immune response. The method involves: (i) providing a test compound; (ii) culturing the compound, together with one or more functional polypeptides and/or functional polypeptide derivatives according to the invention, and a B cell or a T cell, or a B cell or a T cell activating stimulus together; and (iii) determining whether the test compound enhances the response of the T cell to the stimulus, as an indication that the test compound enhances an immune response.

A “B cell activating stimulus”, as used herein, may, for example, be an antibody that binds to CD40. Alternatively, the stimulus may be an anti-IgM antibody or a CD154 molecule.

A “T cell activating stimulus”, as used herein, may, for example, be an antibody that binds to a T cell receptor or a CD3 polypeptide. Alternatively, the stimulus may be an alloantigen or an antigenic peptide bound to a major histocompatibility complex (MHC) molecule on the surface of an antigen presenting cell (APC). The APC can be transfected or transformed with a nucleic acid encoding one or more functional polypeptides and/or functional polypeptide derivatives according to the invention and the functional polypeptide and/or functional polypeptide derivative according to the invention may be expressed on the surface of the APC.

An additional aspect of the present invention encompasses also an ex vivo method. The method can also be an ex vivo procedure that, for example, involves: (i) providing a recombinant cell which is the progeny of a cell obtained from the mammal and which has been transfected of transformed ex vivo with one or more nucleic acids encoding the first co-stimulatory polypeptide and the one or more additional polypeptides so that the cell expresses the first co-stimulatory polypeptide and the one or more additional co-stimulatory polypeptides; and (ii) administering the cell to the mammal. Alternatively, the ex vivo procedure may involve: (i) providing a first recombinant cell which is the progeny of a cell obtained from the mammal and which has been transfected or transformed ex vivo with a nucleic acid encoding the first co-stimulatory polypeptide; providing one or more additional recombinant cells each of which is the progeny of a cell obtained from the mammal and each of which has been transfected or transformed ex vivo with a nucleic acid encoding one of the additional one or more co-stimulatory polypeptides; and (ii) administering the first cell and the one or more additional cells to mammal. The recombinant cells used in the any of the ex vivo methods may be antigen presenting cells (APC) and they may express the first co-stimulatory polypeptide and/or the one or more additional co-stimulatory polypeptides on their surface. Prior to the administering, APC may be pulsed with an antigen or an antigenic peptide. In addition, the cell obtained from the mammal may be a tumor cell. In any of the above methods of co-stimulating a B cell, a T cell, or a B cell and a T cell, the mammal may be suspected of having, for example, an immunodeficiency disease, an inflammatory condition, or an autoimmune disease.

Another important aspect of the present invention relates to a method of treating and/or preventing a disease in a mammal, wherein said disease is selected from autoimmune diseases and diseases that benefit from an enhanced or reduced immune response, preferably type I diabetes and multiple sclerosis, asthma, arthritis, psoriasis, colitis or rejection of transplanted organs, immuno deficiency diseases, and cancer as well as, preferably, selected from autoimmune diseases mediated by antibodies including, and preferably consisting of, myasthenia gravis, arthritis, lupus erythematosus, pemhigus, and rejection of xenotransplants, which method comprises administering to the mammal a therapeutically effective amount of an inventive polypeptide, a functional polypeptide, a functional derivative of a polypeptide, a nucleic acid and/or recombinant vector encoding/expressing an inventive polypeptide, a functional polypeptide and/or a functional derivative of a polypeptide according to the invention.

An additional embodiment of the invention relates to the administration of a pharmaceutical or sterile composition, in conjunction with a pharmaceutically acceptable carrier, for any of the therapeutic effects discussed above. Such pharmaceutical compositions may consist of an inventive polypeptide, a functional polypeptide, a functional derivative of a polypeptide, a nucleic acid and/or recombinant vector encoding/expressing an inventive polypeptide, a functional polypeptide and/or a functional derivative of a polypeptide according to the invention. The compositions may be administered alone or in combination with at least one other agent, such as a stabilizing compound, which may be administered in any sterile, biocompatible pharmaceutical carrier including, but not limited to, saline, buffered saline, dextrose, and water. The compositions may be administered to a patient alone, or in combination with other agents, drugs, or hormones.

The compounds to be administered may be administered by any suitable route of administration as known in the art, such as orally, e.g., in the form of a tablet or capsule, subcutaneously, transdermally, rectally, intravenously, intramuscularly, intra-arterially, intramedullaryly, intrathecally, intraventricularly, intraperitoneally, intranasally, enterally, topically, sublingually, parenterally, e.g., by injection and the like. Preferably, the compound is administered by intramuscular injection. Alternatively, the polypeptide compounds may be administered by the administration of a nucleic acid encoding and expressing said polypeptide. Suitable routes of administering nucleic acids are also known in the art. One of ordinary skill in the art will readily appreciate that one route may have a more immediate effect than another route.

Pharmaceutical formulations suitable for parenteral administration may be formulated in aqueous solutions, preferably in physiologically compatible buffers such as Hanks's solution, Ringer's solution, or physiologically buffered saline. Aqueous injection suspensions may contain substances which increase the viscosity of the suspension, such as sodium carboxymethyl cellulose, sorbitol, or dextran. Additionally, suspensions of the active compounds may be prepared as appropriate oily injection suspensions. Suitable lipophilic solvents or vehicles include fatty oils, such as sesame oil, or synthetic fatty acid esters, such as ethyl oleate, triglycerides, or liposomes. Non-lipid polycationic amino polymers may also be used for delivery. Optionally, the suspension may also contain suitable stabilizers or agents to increase the solubility of the compounds and allow for the preparation of highly concentrated solutions.

For topical or nasal administration, penetrants appropriate to the particular barrier to be permeated are used in the formulation. Such penetrants are generally known in the art.

The pharmaceutical compositions of the present invention may be manufactured in a manner that is known in the art, e.g., by means of conventional mixing, dissolving, granulating, dragee-making, levigating, emulsifying, encapsulating, entrapping, or yophilizing processes.

The pharmaceutical composition may be provided as a salt and can be formed with many acids, including but not limited to, hydrochloric, sulfuric, acetic, lactic, tartaric, malic, and succinic acid. Salts tend to be more soluble in aqueous or other protonic solvents than are the corresponding free base forms. In other cases, the preferred preparation may be a lyophilized powder which may contain any or all of the following: 1 mM to 50 mM histidine, 0.1% to 2% sucrose, and 2% to 7% mannitol, at a pH range of 4.5 to 5.5, that is combined with buffer prior to use.

Preferably, the above mentioned compounds for therapy are administered by intravenous or local application, e.g into a tumor.

When a recombinant vector is administered said vector is selected from the group consisting of an adenoviral vector, an adeno-associated viral vector, a retroviral vector, a Herpes simplex viral vector, a lentiviral vector, a Sindbis viral vector, or a Semliki forest viral vector.

The determination of a “therapeutically effective amount” is well within the capability of those skilled in the art. For any compound, the therapeutically effective amount can be estimated initially either in cell culture assays or in an appropriate animal model. The animal model is also used to achieve a desirable concentration range and route of administration. Such information can then be used to determine useful doses and routes for administration in humans.

A therapeutically effective amount refers to that amount of active agent which ameliorates the symptoms or condition. Therapeutic efficacy and toxicity of such compounds can be determined by standard pharmaceutical procedures in cell cultures or experimental animals (e.g., ED50, the dose therapeutically effective in 50% of the population; and LD50, the dose lethal to 50% of the population). The dose ratio between therapeutic and toxic effects is the therapeutic index, and it can be expressed as the ratio, LD50/ED50. Pharmaceutical compositions which exhibit large therapeutic indices are preferred. The data obtained from cell culture assays and animal studies is used in formulating a range of dosage for human use. The dosage of such compounds lies preferably within a range of circulating concentrations that include the ED50 with little or no toxicity. The dosage varies within this range depending upon the dosage form employed, sensitivity of the patient, and the route of administration.

The exact dosage may be chosen by the individual physician in view of the patient to be treated. Dosage and administration can be adjusted to provide sufficient levels of the active moiety or to maintain the desired effect. Additional factors which may be taken into account include the severity of the disease state (e.g. tumour size and location); age, weight and gender of the patient; diet; time and frequency of administration; drug combination(s); reaction sensitivities; and tolerance/response to therapy. Long acting pharmaceutical compositions can be administered on a daily basis, every 3 to 4 days, every week, or once every two weeks, depending on half-life and clearance rate of the particular formulation.

The mammal may be a guinea pig, dog, cat, rat, mouse, horse, cow, sheep, monkey or chimpanzee. Preferably, the mammal is a human.

A further aspect of the present invention is directed to a method of producing a polypeptide, nucleic acid, or vector according to the invention, wherein a host cell of the invention is cultured and said polypeptide, nucleic acid, or vector is purified. In particular, said method of producing a polypeptide, nucleic acid, or vector of the invention comprises the steps of: (i) providing a host cell of the invention, (ii) culturing said host cell under conditions suitable for expression of said polypeptide, said nucleic acid, or said vector of the invention; and (iii) isolating said polypeptide, nucleic acid, or vector of the invention from said host cell.

In a further aspect of the present invention, a method is provided for producing an antibody according to the invention, said method comprising the steps of: (i) providing a hybridoma cell of the invention or a cell line transfected to express said antibody, (ii) culturing said hybridoma cell or said cell line transfected to express said antibody under conditions suitable for expression of said antibody of the invention; and (iii) isolating said antibody from said hybridoma cell or said cell line.

Unless otherwise defined, all technical and scientific terms used herein have the same meaning as commonly understood by one or ordinary skill in the art to which this invention pertains. Preferred methods and materials are described below, although methods and materials similar or equivalent to those described herein can be used in the practice or testing of the present invention.

### EXAMPLES

The following examples serve to illustrate further the present invention and are not intended to limits its scope in any way.

Short Summary

Using a novel PCR-based strategy, the inventors have identified four cDNA sequences (SEQ ID NOS: 1, 5, 9, 13, and 41) corresponding to five genes encoding novel B7-related molecules (hsB7-H4LV, hsB7-H5, mB7-H5, mB7-H6 and hsB7-H6) (SEQ ID NOs: 2, 6, 10, 14, 42).

Translation of the cDNA sequences indicated the five polypeptides encoded by the five cDNA molecules are type I transmembrane proteins of 315 amino acids (hsB7-H4LV), 430 amino acids (hsB7-H5), 428 amino acids (mB7-H5), 280 amino acids (mB7-H6) and 399 amino acid (hsB7-H6), each containing two immunoglobulin (Ig) domains except mB7-H6 contains only one, a transmembrane (TM) and a cytoplasmic domain (IC).

### EXAMPLE 1

Database Search for B7-Related Genes

Protein sequences of both human and mouse B7 family members including CD80, CD86, B7-H1, B7-H2 and B7-H3 were used for BLAST® (Basic Local Alignment Search Tool) searches. The standard protein-protein BLAST (blastp) similarity search program was used with default values except for the following options: Matrix: BLOSUM 62, Gap costs: Existence 11 and Extension 1 and no low complexity filter. The BLAST results were further screened for hypothetical proteins, unknown proteins and proteins containing the text “similar to” in the definition of the database entry.

These protein sequences were subjected to a further analysis for the occurrence of a catalogue of different features such as particular domains and specified intrinsic features which are included in the SMART (a Simple Modular Architecture Research Tool) programm (Letunic I. et al (2002) Nucleic Acid Res. 30, 242-244). SMART allows the identification and annotation of genetically mobile domains and the analysis of domain architectures. The sequences were analysized for the following criteria, the existence of a signal peptide at the N-terminus, two tandem Ig-domains, transmembrane domain, a short cytoplasmic domain, the absence of a SPRY domain (after SPIa and the Ryanodine receptor) (also called heptad structure and B30.2 domain) at the C-terminal portion of the cytoplasmic domain. Furthermore, the membrane distal Ig domain must belong to the immunoglobulin V-type whereas the membrane proximal Ig domain should belong to the C-type family or at least be an Ig-like domain. The immunoglobulin V-type domain contributes to the noncovanlent dimer interface (Ikemizu S. et al. (2000) Immunity 12, 51-60). More recently, two independent crystallographic analyses provided the first structural description of the CTLA-4-B7 costimulatory complex (Schwartz J. C. et al. (2001) Nature 410, 604-608; Stamper C. C. et al. (2001) Nature 410, 608-611). The complex showed the involvement of the Ig V-type domain of human B7-2 in receptor-binding. Therefore, the distal Ig domain must belong to the Ig V-type domain.

Five potential hypothetical cDNA sequences were obtained with the above searches which either completely or partially met the criteria for the above described B7 family members.

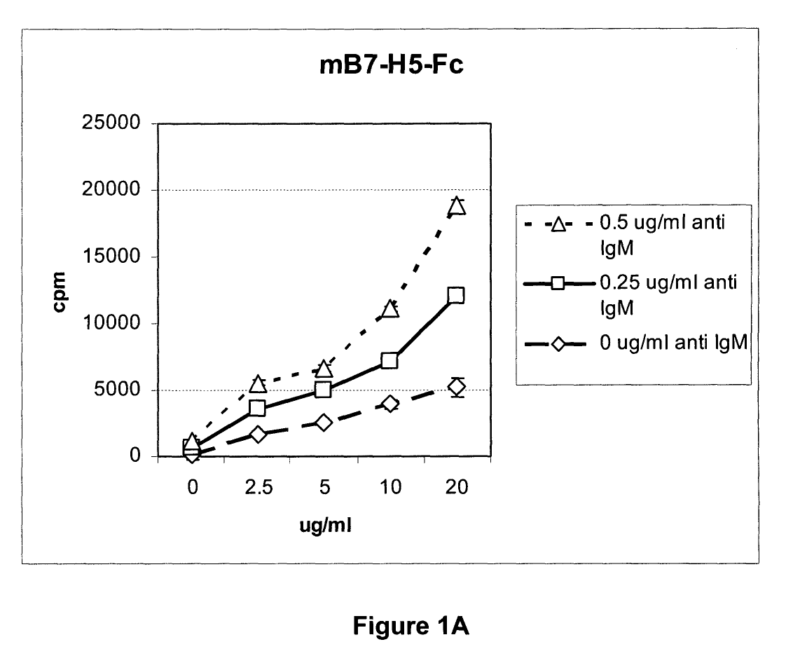

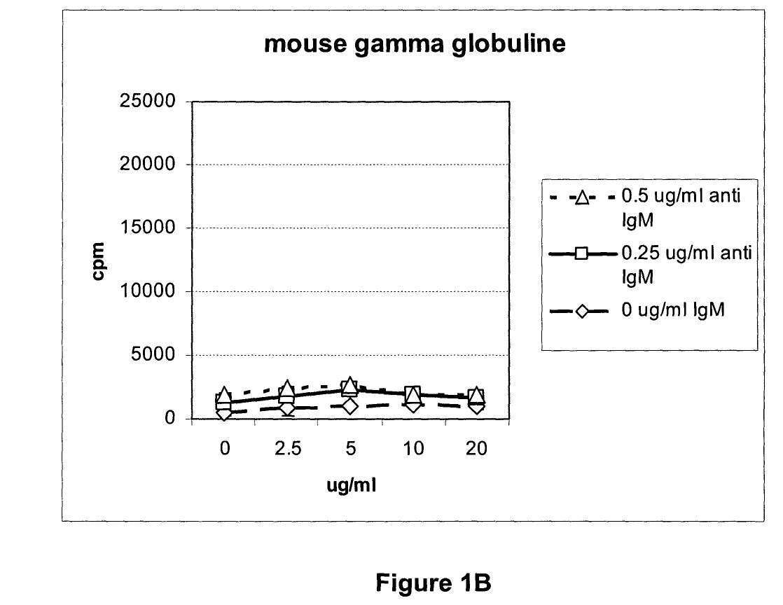

One result of the bioinformatical analysis was a hypothetical protein (Accession number XP_{—}087714) which met all terms. The nucleic acid sequence of said hypothetical protein was confirmed by analysis of independent reverse transcription-polymerase chain reaction (RT-PCR) products from human normal spleen poly(A)+ RNA and also human testis total RNA as described in example 2. This sequence (SEQ ID NO:1) is designated hsB7-H4LV and encodes a putative 315 amino acids (aas) protein and shares identity in its predicted extracellular receptor-binding domains with human CD80 (18%), CD86 (21%), B7-H1 (18%), B7-H2 (18%), B7-H3 (29%) (see FIG. 1).

The putative hsB7-H4LV protein contains a signal peptide in its NH₂-terminus ranging from 1-35 aas, a single extracellular Ig domain (E-value 2.70e-06) ranging form 44-151 aas, a single extracellular Ig-like domain (E-value 3.00e-13) ranging from 159-244 aas, a transmembrane region ranging from 258-277 aas, and a 38-aas cytoplasmic tail (SEQ ID NO: 2).

A second hypothetical protein (Accession number XP_{—}087460) was found which contains the particular Ig domains and a signal peptide. However, the transmembrane domain and cytoplasmic tail is missing. The amino acid sequence of XP_{—}087460 was used for a homology search using an EST database. The obtained homologe EST sequences were aligned and the consensus sequence was used to complete the C-terminus of XP_{—}087460. Thereby a virtual cDNA, designated hsB7-H5 (SEQ ID NO: 5), was designed and its existence was confirmed by RT-PCR (as described in example 4). This sequence (SEQ ID NO: 5) encodes a putative 430 aas protein (SEQ ID NO: 6) and shares an identity in its predicted extracellular receptor-binding domain with human CD80 (18%), CD86 (24%), B7-H1 (18%), B7-H2 (17%), B7-H3 (22%), B7-H4 (19%) (Table 1).

The putative hsB7-H5 protein contains a signal peptide in its NH₂-terminus ranging form 1-15 aas, a single extracellular Ig V-type domain (E-value 6.97e-03) ranging from 28-142 aas, a single extracellular Ig C2-type domain (E-value 2.37e-05) ranging from 155-221 aas, a transmembrane region ranging from 245-267 aas, and a 163-aas cytoplasmic tail (SEQ ID NO: 6)

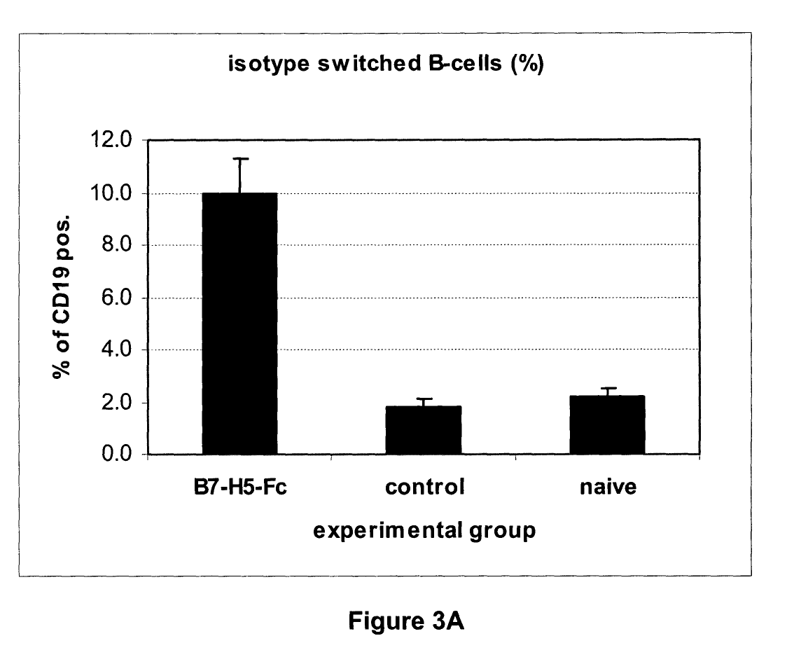

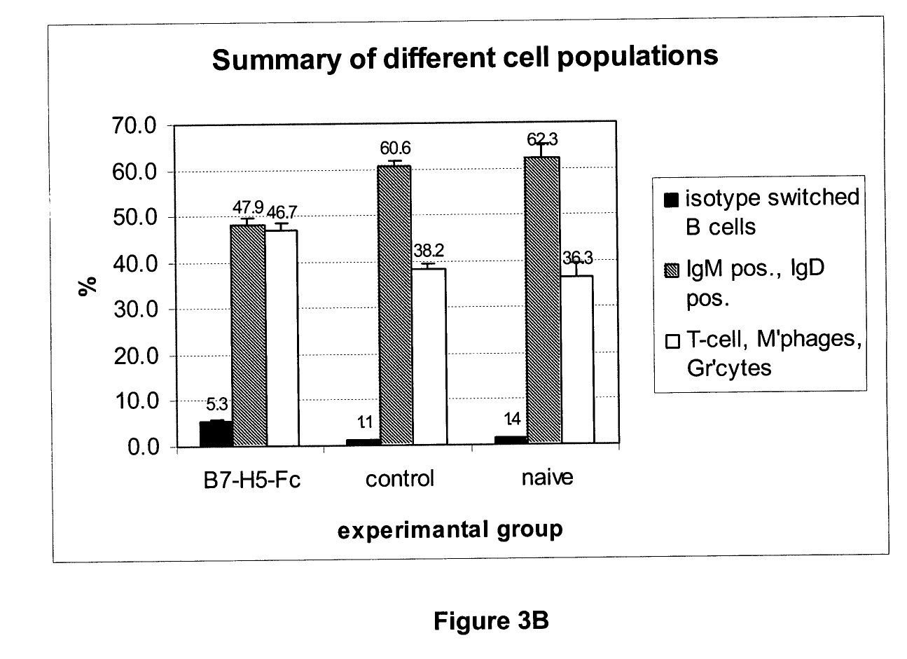

The third hypothetical protein was a putative mouse orthologe (Acc. No XM_{—}156112) of XP_{—}087460 which was found using the standard protein-protein BLAST (blastp) similarity search program and the IgG domains of the XP_{—}087460 as query sequence in the NCBI database. However, this mouse orthologe was a hypothetical protein and the integrity of the 5′ end and 3′ end had to be experimentally confirmed. A search for ESTs (expressed sequence tags) using the derived amino acid sequence of mB7-H5 as query resulted in several identical hits coding for the IgG domain regions whereas the N-terminus and C-terminus showed no similarity to the found ESTs. An alignment of the hsB7-H5 and its mouse orthologe XM_{—}156112 showed a variation within the 5′end and 3′ end. Therefore, with the help of the mouse EST database sequences, mouse genomic database sequences, and hsB7-H5, a virtual mouse orthologe of hsB7-H5 cDNA was designed (FIG. 3). The sequence of this virtual mouse orthologe, designated mB7-H5 (SEQ ID NO: 9), encodes a putative 428 aas protein (SEQ ID NO: 10) and is 89% identical to hsB7-H5. The existence of mB7-H5 was confirmed by RT-PCR and DNA sequencing (as described in example 6).

The putative mB7-H5 protein contains a signal peptide in its NH₂-terminus ranging from 1-23 aas, a single extracellular Ig V-type domain ranging from 39-122 aas, a single extracellular Ig C2-type domain ranging from 156-222 aas, a transmembrane region ranging from 240-262 aas, and a 166-aas cytoplasmic tail (SEQ ID NO: 10).

In a similar approach the sequence encoding mB7-H6 protein was found. The existence of the mB7-H6 was confirmed by RT-PCR and DNA sequencing (as described in example 8). This sequence (SEQ ID NO: 13) encodes a putative 280 aas protein (SEQ ID NO: 14) and shares an identity in its predicted extracellular receptor-binding domain with mouse CD80 (16%), CD86 (14%), B7-H1 (18%), B7-H2 (19%), B7-H3 (20%), B7-H5 (17%) (see FIG. 1). The putative mB7-H6 protein contains a signal peptide in its NH₂-terminus ranging from 1-20 aas, however only a single extracellular Ig V-type domain ranging from 34-115 aas, a transmembrane region ranging from 188-210 aas, and a 70-aas cytomplasmic tail (SEQ ID NO: 14).

The hsB7-H6 protein was found by a standard protein-protein BLAST (blastp) similarity search using the mB7-H6 as query sequence. The existence of the hsB7-H6 was confirmed by RT-PCR and DNA sequencing (as described in example 19). This sequence (SEQ ID NO: 41) encodes a putative 399 aas protein (SEQ ID NO: 42) and shares an identity in its predicted extracellular receptor-binding domain with human CD80 (20%), CD86 (19%), B7-H1 (17%), B7-H2 (20%), B7-H3 (21%), B7-H4 (18%) and B7-H5 (20%) (see FIG. 1). The putative hsB7-H6 protein contains a signal peptide in its NH₂-terminus ranging from 1-19 aas, a single extracellular Ig V-type domain ranging from 36-115 aas, a single extracellular Ig C2-type domain ranging from 157-218 aas, a transmembrane region ranging from 284-303 aas, and a 105-aas cytoplasmic tail (SEQ ID NO: 42).

TABLE 1
Percentage of identity on amino acid level of the ectodomain of different B7-family
members of human (h) and mouse (m) species.

| | | | | hB7- | mB7- | hB7- | mB7- | hB7- | mB7- | hB7- | hB7- | mB7- | hB7- | mB7- |
|---|---|---|---|---|---|---|---|---|---|---|---|---|---|---|
| | mCD80 | hCD86 | mCD86 | H1 | H1 | H2 | H2 | H3 | H3 | H4 | H5 | H5 | H6 | H6 |
| hCD80 | 48 | 26 | 23 | 20 | 20 | 22 | 25 | 25 | 26 | 18 | 18 | 18 | 20 | 14 |
| mCD80 | | 29 | 26 | 23 | 21 | 22 | 24 | 25 | 25 | 19 | 20 | 20 | 19 | 16 |
| hCD86 | | | 56 | 18 | 23 | 20 | 26 | 23 | 24 | 21 | 24 | 22 | 19 | 16 |
| mCD86 | | | | 20 | 20 | 22 | 23 | 24 | 26 | 20 | 21 | 22 | 20 | 14 |
| hB7- | | | | | 70 | 21 | 22 | 29 | 29 | 18 | 17 | 16 | 17 | 15 |
| H1 | | | | | | | | | | | | | | |
| mB7- | | | | | | 22 | 22 | 29 | 29 | 18 | 18 | 20 | 17 | 18 |
| H1 | | | | | | | | | | | | | | |
| hB7- | | | | | | | 48 | 30 | 29 | 19 | 19 | 20 | 19 | 17 |
| H2 | | | | | | | | | | | | | | |
| mB7- | | | | | | | | 28 | 27 | 18 | 20 | 21 | 21 | 19 |
| H2 | | | | | | | | | | | | | | |
| hB7- | | | | | | | | | 92 | 29 | 22 | 23 | 21 | 19 |
| H3 | | | | | | | | | | | | | | |
| mB7- | | | | | | | | | | 27 | 23 | 23 | 21 | 20 |
| H3 | | | | | | | | | | | | | | |
| hB7- | | | | | | | | | | | 19 | 19 | 18 | 14 |
| H4LV | | | | | | | | | | | | | | |
| hB7- | | | | | | | | | | | | 89 | 20 | 17 |
| H5 | | | | | | | | | | | | | | |
| mB7- | | | | | | | | | | | | | 21 | 17 |
| H5 | | | | | | | | | | | | | | |
| hB7- | | | | | | | | | | | | | | 44 |
| H6 | | | | | | | | | | | | | | |

### EXAMPLE 2

Molecular Cloning of the Human hsB7-H4LV

For the cDNA synthesis 5 μg human testis total RNA, purchased from CLONTECH Laboratories, Inc. Palo Alto, Calif. (Cat. No. 64027-1), and 0.5 μg human normal spleen poly(A)+ RNA, purchased from Invitrogen life technologies, USA, (Cat. No. D6117-15), were used. The 1^{st} strand cDNA was synthesized in a reaction containing 50 mM Tris-HCl, pH 8.3, 75 mM KCl, 3 mM MgCl₂, 10 mM dithiothreitol, 500 μM dATP, dCTP, dGTP, dTTP, 25 μg/ml oligo(dT)12-18, 40 Units RNaseOUT (Invitrogen life technologies, Cat. No. 10777-019), and 200 Units SUPERSCRIPT™ II RNase H reverse transcriptase (Invitrogen life technologies, Cat. No. 18064-022) in a total volume of 20 μl at 42° C. for 1 hour. Following the reverse transcription the reaction was terminated by incubation at 85° C. for 5 minutes. To remove the complementary RNA prior to PCR the cDNA was treated with 2 units of RNase H at 37° C. for 30 minutes.

The cDNA sequence of B7-H4LV containing the complete open reading frame was amplified by PCR. The PCR was performed using either the normal spleen cDNA or the testis cDNA as template as well as the High Fidelity PCR System composed of a unique enzyme mix containing thermostable Taq DNA polymerase, a proofreading polymerase (Roche, Cat. No. 1 732 650), and the primers LV43-XM087714f (5′-TGC TGA CGA GAG ATG GTG G-3′) (SEQ ID NO: 25) and LV44-XM087714b (5′-CCA CAG CCT TTA GAT GAC GG-3′) (SEQ ID NO: 26). The PCR product (968 base pairs) of B7-H4LV obtained from the testis cDNA was cloned into pGEM-T plasmid using T4 DNA ligase (Promega, Cat. No. A3600). After ligation the plasmid was used to transform competent E. coli strain XL 1-Blue. The nucleic acid sequence of B7-H4LV (SEQ ID NO: 1) was verified by DNA sequencing of two independent clones.

### EXAMPLE 3

Preparation and Purification of Soluble (Secreted) Form of hsB7-H4LV Protein

Production of Soluble hsB7-H4LV

In order to produce large amount of soluble hsB7-H4LV, a plasmid encoding a secreted form of B7-H4LV fused to the Fc constant region of human IgG1 or a FLAG tagged rat comp pentamerisation domain was introduced into eukaryotic cell and hsB7-H4LV expressing cells were selected using geneticin.

In more detail, a DNA fragment encoding a secreted form of hsB7-H4LV was constructed by polymerase chain reaction (PCR) as follow: The original hsB7-H4LV cDNA clone in pGEM-T (SEQ ID NO: 1) was used as template. The PCR reaction was performed using the High Fidelity PCR System composed of unique enzyme mix containing termostable Taq DNA polymerase and a proofreading polymerase (Roche, Cat. No. 1 732 650), and 10 picomoles each of a sense and an antisense oligonucleotide primer in a final volume of 50 microliters. The sense oligonucleotide primer, designated LV49-XM087714f, had the sequence 5′-GGG GGT ACC TGC TGA CGA GAG ATG GTG-3′ (SEQ ID NO: 27) and contained the recognition site for the restriction enzyme KpnI (GGTACC), the strong translation initiation site (GAGAGATGG), and was identical to the hsB7-H4LV cDNA from nucleotides 2 to 20 (SEQ ID NO: 1). The antisense designated LV48-XM087714b had the sequence 5′-CGG CTA GCC CGG GTA CGA ACA CGT C-3′ (SEQ ID NO: 28) and contained the recognition site for the restriction enzyme NheI (GCTAGC) to fuse to the Fc constant region of human IgG1 and was identical, in an antisense orientation, to the hsB7-H4LV cDNA from nucleotides 750 to 766 (SEQ ID NO: 1).

The PCR reaction was performed on a Hybaid programmable thermal cycler with 5 cycles of 94°, 30 sec, 57°, 45 sec, 68°, 70 sec, and 25 cycles of 94°, 30 sec, 68°, 70 sec and a final cycle of 72°, 7 min. The resulting PCR product extending from hsB7-H4LV nucleotide 2-766 was flanked by restriction sites. In the cell, this DNA encoded a secreted form of the hsB7-H4LV protein from methionine amino acid 1 to glycin amino acids 251 (SEQ ID NO: 1). The PCR product was cloned into pGEM-T and the sequence was confirmed by sequencing both strands.

The plasmid DNA was digested with KpnI and NheI and the insert containing the nucleic acid molecule encoding for the extracellular domain (ECD) of hsB7-H4LV (SEQ ID NO: 3) was ligated into each pCEP-SP-Xa1-Fc* and pCEP-comp-FL-C expression vector. Both vectors were derivatives of the episomal mammalian expression vector pCEP4 (Invitrogen), carrying the Epstein-Barr Virus replication origin (oriP) and nuclear antigen (encoded by the EBNA-1 gene) to permit extrachromosomal replication, and contained a Puromycin selection marker in place of the original Hygromycin B resistance gene.

The pCEP-SP-Xa1-Fc* is an expression vector that contained a KpnI cloning site downstream of the strong cytomegalo virus (CMV) promoter, a NheI cloning site upstream of the Factor X protease recognition site flanking the N-terminus of the Fc constant region of the human IgG1 and a SV40 poly(A) signal necessary for expression in mammalian cells. In addition, the vector contained the EBNA, origin of replication, ampilicin resistance gene, puromycin resistance gene for the selection of cells producing the fusion protein. The resulting plasmid, pCEP-hsB7-H4LV(ECD)-Fc (SEQ ID NO: 17), drove the expression of a B7-H4LV (ECD)-Fc domain fusion protein under the control of a CMV promoter.

The pCEP-comp-FL-C was identical to pCEP-SP-Xa1-Fc* except that the nucleic acid sequence encoding for SP-Xa1-Fc* part was replaced by nucleic acid sequences encoding for the rat pentamerizaion domain containing FLAG (FL) tag at the C terminus. The resulting plasmid pCEP-hsB7-H4LV(ECD)-comp-FL-C (SEQ ID NO: 18) drove the expression of hsB7-H4LV (ECD) fused to the C-terminal FLAG tagged rat comp pentamerisation domain under the control of a CMV promoter.

Expression of the hsB7-H4LV (ECD)-Fc domain and the hsB7-H4LV (ECD)-comp-Flag domain fusion protein was performed in EBNA cells (Invitrogen). One day before transfection, 5×10⁶ EBNA cells were plated onto a 10 cm tissue culture plate. Cells were then transfected with pCEP-hsB7-H4LV(ECD)-Fc (SEQ ID NO: 17)-or pCEP-hsB7-H4LV(ECD)-comp-FL-C (SEQ ID NO: 18) using Lipofectamin Plus (Invitrogen), incubated one day, and subjected to selection in the presence of 1 μg/ml puromycin. After 24 hours of selection, puromycin-resistant cells were transferred to a Poly-L-Lysine coated 15 cm tissue culture plate and grown to confluency. Medium was replaced by serum-free medium and the supernatant containing the hsB7-H4LV(ECD)-Fc fusion protein or hsB7-H4LV(ECD)-comp-FL-C fusion protein, respectively, was collected every 3 days.

Pooled supernatants of hsB7-H4LV(ECD)-Fc fusion protein expressing cells were filtered through a 0.22 μM Millex GV sterile filter (Millipore) and applied to a protein A-sepharose column. The column was washed with 5 column volumes of 20 mM Tris pH 8.0, 150 mM NaCl, and bound protein was eluted with citrate-phosphate buffer pH 3.6. 1 ml fractions were collected in tubes containing 0.1 ml of 0.5 M Na₂HPO₄ for neutralization. Positive fractions were identified by SDS-PAGE and pooled. The buffer was exchanged with phosphate-buffered saline (PBS) by ultrafiltration through Ultrafree Biomax 10k (Millipore). The purified protein in PBS was then filtered through 0.22 μM Millex GV sterile filters (Millipore) and stored at 4° C.

Pooled supernatants of hsB7-H4LV(ECD)-comp-FLAG fusion protein expressing cells were filtered through a 0.22 μM Millex GV sterile filter (Millipore) and applied to an affinity column containing ANTI-FLAG M2-agarose (Sigma, Cat. No A2220). The column was washed with 10 column volumes of phosphate-buffered saline (PBS) and bound FLAG fusion protein was eluted with five one-column volumes of a solution containing 100 μg/ml FLAG peptide (Sigma, Cat No F3290) in TBS. 1 ml fractions were collected and positive fractions were identified by SDS-PAGE and pooled. The buffer and free FLAG peptides were exchanged with phosphate-buffered saline (PBS) by ultrafiltration through Ultrafree Biomax 10k (Millipore). The purified protein in PBS was then filtered through 0.22 μM Millex GV sterile filters (Millipore) and stored at 4° C.

### EXAMPLE 4

Molecular Cloning of the Human hsB7-H5

For the cDNA synthesis 5 μg human testis total RNA purchased from CLONTECH Laboratories, Inc. Palo Alto, Calif. (Cat. No. 64027-1) was used. The 1^{st} strand cDNA was synthesized in a reaction containing 50 mM Tris-HCl, pH 8.3, 75 mM KCl, 3 mM MgCl₂, 10 mM dithiothreitol, 500 μM dATP, dCTP, dGTP, dTTP, 25 μg/ml oligo(dT)12-18, 40 Units RNaseOUT (Invitrogen life technologies, Cat. No. 10777-019), and 200 Units SUPERSCRIPT™ II RNase H⁻ reverse transcriptase (Invitrogen life technologies, Cat. No. 18064-022) in a total volume of 20 μl at 42° C. for 1 hour. Following the reverse transcription the reaction was terminated by incubation at 85° C. for 5 minutes. To remove the complementary RNA prior to PCR the cDNA was treated with 2 units of RNase H at 37° C. for 30 minutes.

The cDNA sequence of hsB7-H5 containing the complete open reading frame was amplified by PCR. The PCR was performed using the testis cDNA as template, High Fidelity PCR System composed of a unique enzyme mix containing termostable Taq DNA polymerase and a proofreading polymerase (Roche, Cat. No. 1 732 650), and the primers LV50-XP087460f (5′-TTT CCA TCT GAG GCA AGA AG-3′) (SEQ ID NO: 29) and LV60-hsB7-H5b (5′-TTC CTC ATG TCC TAT ACC AAG G-3′) (SEQ ID NO: 30). The PCR product of hsB7-H5 obtained from the testis cDNA was cloned into pGEM-T plasmid using T4 DNA ligase (Promega, Cat. No. A3600). No PCR product was detected using brain and spleen derived cDNA. After ligation the plasmid was used to transform competent E. coli strain XL1-Blue. The nucleic acid sequence of hsB7-H5 (SEQ ID NO: 5) was verified by DNA sequencing of two independent clones.

### EXAMPLE 5

Preparation and Purification of Soluble (Secreted) Form of hsB7-H5 Protein

Production of Soluble hsB7-H5

In order to produce large amount of soluble hsB7-H5, a plasmid encoding a secreted form of hsB7-H5 fused to the Fc constant region of human IgG1 or the FLAG tagged rat comp pentamerisation domain was introduced into eukaryotic cell and hsB7-H5 expressing cells were selected using geneticin.

In more detail, a DNA fragment encoding a secreted form of hsB7-H5, designated B7-H5 (ECD), was constructed by polymerase chain reaction (PCR) as follow: The full length hsB7-H5 cDNA clone in pGEM-T (described in example 4) was used as template. The PCR reaction was performed using the High Fidelity PCR System composed of a unique enzyme mix containing termostable Taq DNA polymerase and a proofreading polymerase (Roche, Cat. No. 1 732 650), and 10 picomoles each of a sense and an antisense oligonucleotide primer in a final volume of 50 microliters. The sense oligonucleotide primer, designated LV56-sec-hsB7-H5f, had the sequence 5′-GG GGT ACC ATG TCT CTG GTG GAA CTT TTG C-3′ (SEQ ID NO: 31) and contained the recognition site for the restriction enzyme KpnI (GGTACC), the strong translation initiation site (GTACCATG) and was identical to the hsB7-H5 cDNA from nucleotides 175 to 196 (SEQ ID NO:5). The antisense designated LV57-sec-hsB7-H5b had the sequence 5′-C GGC TAG CCC AAT GTT CCT GGG CTG G-3′ (SEQ ID NO: 32) and contained the recognition site for the restriction enzyme NheI (GCTAGC) to fuse to the Fc constant region of human IgG1 or comp-FLAG domain and is identical, in an antisense orientation, to the B7-H5 cDNA from nucleotides 876 to 893 (SEQ ID NO:5).

The PCR reaction was performed on a Hybaid programmable thermal cycler with 5 cycles of 94°, 30 sec, 58°, 45 sec, 72°, 70 sec, and 25 cycles of 94°, 30 sec, 72°, 70 sec and a final cycle of 72°, 7 min. The resulting PCR product which extended from hsB7-H5 nucleotide 175-893 was flanked by restriction sites. In the cell, this DNA encodes a secreted form of the hsB7-H5 protein from methionine amino acid 1 to glycin amino acid 240 (SEQ ID NO:5). The PCR product was cloned into pGEM-T and the sequence confirmed by sequencing both strands.

The plasmid DNA was digested with KpnI and NheI and the insert, containing the nucleic acid molecule encoding for the extracellular domain (ECD) of hsB7-H5 (SEQ ID NO: 7), was ligated into each pCEP-SP-Xa1-Fc* and pCEP-comp-FL-C expression vector. Both vectors were derivatives of the episomal mammalian expression vector pCEP4 (Invitrogen), carrying the Epstein-Barr Virus replication origin (orip) and nuclear antigen (encoded by the EBNA-1 gene) to permit extrachromosomal replication, and contained a Puromycin selection marker in place of the original Hygromycin B resistance gene.

The pCEP-SP-Xa1-Fc* is an expression vector that contains a KpnI cloning site downstream of the strong cytomegalo virus (CMV) promoter, a NheI cloning site upstream of the Factor X protease recognition site flanking the N-terminus of the Fc constant region of the human IgG1 and a SV40 poly(A) signal necessary for expression in mammalian cells. In addition, the vector contains the EBNA, origin of replication, ampilicin resistance gene, puromycin resistance gene for the selection of cells producing the fusion protein. The resulting plasmid pCEP-hsB7-H5(ECD)-Fc (SEQ ID NO: 19) drove the expression of a hsB7-H5 (ECD)-Fc domain fusion protein under the control of a CMV promoter.

The pCEP-comp-FL-C was identical to pCEP-SP-Xa1-Fc* except that the nucleic acid sequence encoding for SP-Xa1-Fc* part was replaced by nucleic acid sequences encoding for the rat comp pentamerization domain fused with a C-terminal FLAG tag. The resulting plasmid pCEP-hsB7-H5(ECD)-comp-FL-C (SEQ ID NO: 20) drove the expression of a hsB7-H5 (ECD) fused to “comp” pentamerizaion domain containing FLAG (FL) tag at the C terminus under the control of a CMV promoter.

Expression and purification of the hsB7-H5 (ECD)-Fc domain and the hsB7-H5 (ECD)-comp-Flag domain fusion protein were performed according detailed descriptions in example 3.

### EXAMPLE 6

Molecular Cloning of the Mouse B7-H5

For the PCR cDNA libraries of different mouse tissues (e.g. brain, spleen, liver, lung) cloned into the pDEL expression vector were used as template.

The cDNA sequence of mB7-H5 containing the complete open reading frame was amplified by PCR. The PCR was performed using pDEL library containing mouse liver cDNA as template, High Fidelity PCR System composed of a unique enzyme mix containing termostable Taq DNA polymerase and a proofreading polymerase (Roche, Cat. No. 1 732 650), and the primers JS7-mB7-H5f (5′-atg act cgg cgg cgc tc-3′) (SEQ ID NO: 33) and JS8-mB7-H5r (5′-cta tac cag gga ccc tgc tcg-3′) (SEQ ID NO: 34). The PCR product of mB7-H5 obtained from the liver cDNA was cloned into pCR II TOPO plasmid using T4 DNA ligase. No PCR product was detected using brain and spleen derived cDNA. After ligation the plasmid was used to transform competent E. coli strain XL1-Blue. The nucleic acid sequence of mB7-H5 (SEQ ID NO: 9) was verified by DNA sequencing of four independent clones.

### EXAMPLE 7

Preparation and Purification of Soluble (Secreted) Form mB7-H5 Protein

Production of Soluble mB7-H5

In order to produce large amounts of soluble mB7-H5, a plasmid encoding a secreted form of mB7-H5 fused to the Fc constant region of human IgG1 or the FLAG tagged rat comp pentamerisation domain was introduced into eukaryotic cell and hsB7-H5 expressing cells were selected using geneticin.

In more detail, a DNA fragment encoding a secreted form of mB7-H5, designated mB7-H5 (ECD), was constructed by polymerase chain reaction (PCR) as follow: The full length mB7-H5 cDNA clone in pCR II TOPO (described in example 6) was used as template. The PCR reaction was performed using the High Fidelity PCR System composed of a unique enzyme mix containing termostable Taq DNA polymerase and a proofreading polymerase (Roche, Cat. No. 1 732 650), and 10 picomoles each of a sense and an antisense oligonucleotide primer in a final volume of 50 microliters. The sense oligonucleotide primer, designated MSt-1mB7-H5for, had the sequence 5′-GGG GTA CCA TGA CTC GGC GGC GCT CC-3′ (SEQ ID NO: 35) and contained the recognition site for the restriction enzyme KpnI (GGTACC), the strong translation initiation site (GTACCATG) and was identical to the mB7-H5 cDNA from nucleotides 64 to 81 (SEQ ID NO:9). The antisense designated MSt-2 mB7-H5rev had the sequence 5′-GGG CTA GCA CGG GTG AGA TAA CCT GGA G-3′ (SEQ ID NO: 36) and contained the recognition site for the restriction enzyme NheI (GCTAGC) to fuse to the Fc constant region of human IgG1 or comp-FLAG domain and is identical, in an antisense orientation, to the mB7-H5 cDNA from nucleotides 751 to 768 (SEQ ID NO:9).

The PCR reaction was performed on a Hybaid programmable thermal cycler with 5 cycles of 94°, 30 sec, 58°, 45 sec, 72°, 70 sec, and 25 cycles of 94°, 30 sec, 72°, 70 sec and a final cycle of 72°, 7 min. The resulting PCR product which extended from mB7-H5 nucleotide 64-768 was flanked by restriction sites. In the cell; this DNA encodes a secreted form of the mB7-H5 protein from methionine amino acid 1 to prolin amino acid 235 (SEQ ID NO:9). The PCR product was cloned into pGEM-T and the sequence confirmed by sequencing both strands.

The plasmid DNA was digested with KpnI and NheI and the insert, containing the nucleic acid molecule encoding for the extracellular domain (ECD) of mB7-H5 (SEQ ID NO: 11), was ligated into each pCEP-SP-Xa1-Fc* and pCEP-comp-FL-C expression vector. Both vectors were derivatives of the episomal mammalian expression vector pCEP4 (Invitrogen), carrying the Epstein-Barr Virus replication origin (oriP) and nuclear antigen (encoded by the EBNA-1 gene) to permit extrachromosomal replication, and contained a Puromycin selection marker in place of the original Hygromycin B resistance gene.

The pCEP-SP-Xa1-Fc* is an expression vector that contains a KpnI cloning site downstream of the strong cytomegalo virus (CMV) promoter, a NheI cloning site upstream of the Factor X protease recognition site flanking the N-terminus of the Fc constant region of the human IgG1, and a SV40 poly(A) signal necessary for expression in mammalian cells. In addition, the vector contains the EBNA, origin of replication, ampilicin resistance gene, puromycin resistance gene for the selection of cells producing the fusion protein. The resulting plasmid pCEP-mB7-H5(ECD)-Fc (SEQ ID NO: 21) drives expression of the mB7-H5 (ECD)-Fc domain fusion protein under the control of a CMV promoter.

The pCEP-comp-FL-C was identical to pCEP-SP-Xa1-Fc* except that the nucleic acid sequence encoding for SP-Xa1-Fc* part was replaced by nucleic acid sequences encoding for comp pentamerization domains containing a C-terminal Flag tag. The resulting plasmid pCEP-mB7-H5-comp-FL-C (SEQ ID NO: 22) drives expression of mB7-H5 (ECD) fused to rat “comp” pentamerizaion domain containing FLAG (FL) tag at the C terminus under the control of a CMV promoter.

Expression and purification of the mB7-H5 (ECD)-Fc domain and the mB7-H5 (ECD)-comp-Flag domain fusion protein were performed according detailed descriptions in example 3.

### EXAMPLE 8

Molecular Cloning of the Mouse B7-H6

For the cDNA synthesis 4 μg mouse macrophage total RNA was used. The total RNA was obtained by using RNeasy MiniPrep (Qiagen; Cat. No. 74104) and isolated mouse macrophages. The 1^{st} strand cDNA was synthesized in a reaction containing 50 mM Tris-HCl, pH 8.3, 75 mM KCl, 3 mM MgCl₂, 10 mM dithiothreitol, 500 μM DATP, dCTP, dGTP, dTTP, 25 μg/ml oligo(dT)12-18, 40 Units RNaseOUT (Invitrogen life technologies, Cat. No. 10777-019), and 200 Units SUPERSCRIPT™ II RNase H⁻ reverse transcriptase (Invitrogen life technologies, Cat. No. 18064-022) in a total volume of 20 μl at 42° C. for 1 hour. Following the reverse transcription the reaction was terminated by incubation at 85° C. for 5 minutes. To remove the complementary RNA prior to PCR the cDNA was treated with 2 units of RNase H at 37° C. for 30 minutes.

The cDNA sequence of mB7-H6 containing the complete open reading frame was amplified by PCR. The PCR was performed using either the mouse macrophage derived cDNA as template as well as the High Fidelity PCR System composed of a unique enzyme mix containing thermostable Taq DNA polymerase, a proofreading polymerase (Roche, Cat. No. 1 732 650), and the primers LV80-mC18f (5′-GTA GCT TCA AAT AGG ATG GAG-3′) (SEQ ID NO: 37) and LV81-mC18b (5′-AAA CTG TGT TCA GCA GGC AG-3′) (SEQ ID NO: 38). The PCR product (867 base pairs) of mB7-H6 obtained from the mouse macrophage cDNA was cloned into pGEM-T plasmid using T4 DNA ligase (Promega, Cat. No. A3600). After ligation the plasmid was used to transform competent E. coli strain XL 1-Blue. The nucleic acid sequence of mB7-H6 (SEQ ID NO: 13) was verified by DNA sequencing of four independent clones.

### EXAMPLE 9

Preparation and Purification of Soluble (Secreted) Form of mB7-H6 Protein

Production of Soluble mB7-H6

In order to produce large amount of soluble mB7-H6 protein, a plasmid encoding a secreted form of mB7-H6 fused to the Fc constant region of human IgG1 or the FLAG tagged rat comp pentamerisation domain was introduced into eukaryotic cell and mB7-H6 expressing cells were selected using geneticin.

In more detail, a DNA fragment encoding a secreted form of mB7-H6, designated mB7-H6 (ECD) (SEQ ID NO: 15), was constructed by polymerase chain reaction (PCR) as follow: The full length mB7-H6 cDNA clone in pGEM-T easy (described in example 8) was used as template. The PCR reaction was performed using the High Fidelity PCR System composed of a unique enzyme mix containing termostable Taq DNA polymerase and a proofreading polymerase (Roche, Cat. No. 1 732 650), and 10 picomoles each of a sense and an antisense oligonucleotide primer in a final volume of 50 microliters. The sense oligonucleotide primer, designated LV82-mC18f, had the sequence 5′-GGG TAC CAG GAT GGA GAT CTC ATC AG-3′ (SEQ ID NO: 39) and contained the recognition site for the restriction enzyme KpnI (GGTACC), the strong translation initiation site (CCAGGATGG) and was identical to the mouse mB7-H6 cDNA from nucleotides 13 to 31 (SEQ ID NO:7). The antisense designated LV83-mC18b had the sequence 5′-GGC TAG CAG GTT CCT CCC TGA AC-3′ (SEQ ID NO: 40) and contained the recognition site for the restriction enzyme NheI (GCTAGC) to fuse to the Fc constant region of human IgG1 or comp-FLAG domain and is identical, in an antisense orientation, to the mB7-H6 cDNA from nucleotides 557 to 574 (SEQ ID NO: 13).

The PCR reaction was performed on a Hybaid programmable thermal cycler with 5 cycles of 94°, 30 sec, 50°, 45 sec, 72°, 60 sec, and 25 cycles of 94°, 30 sec, 72°, 70 sec and a final cycle of 72°, 7 min. The resulting PCR product which extended from mB7-H6 nucleotide 13-574 was flanked by restriction sites. In the cell, this DNA encodes a secreted form of the mB7-H6 protein from methionine amino acid 1 to leucin amino acid 186 (SEQ ID NO: 15). The PCR product was cloned into pGEM-T easy and the sequence confirmed by sequencing both strands.

The plasmid DNA was digested with KpnI and NheI and the insert, containing the nucleic acid molecule encoding for the extracellular domain (ECD) of mB7-H6 (SEQ ID NO: 15), was ligated into each pCEP-SP-XaI-Fc* and pCEP-comp-FL-C expression vector. Both vectors were derivatives of the episomal mammalian expression vector pCEP4 (Invitrogen), carrying the Epstein-Barr Virus replication origin (oriP) and nuclear antigen (encoded by the EBNA-1 gene) to permit extrachromosomal replication, and contained a Puromycin selection marker in place of the original Hygromycin B resistance gene.

The pCEP-SP-Xa1-Fc* is an expression vector that contains a KpnI cloning site downstream of the strong cytomegalo virus (CMV) promoter, a NheI cloning site upstream of the Factor X protease recognition site flanking the N-terminus of the Fc constant region of the human IgG1 and a SV40 poly(A) signal necessary for expression in mammalian cells. In addition, the vector contains the EBNA, origin of replication, ampilicin resistance gene, puromycin resistance gene for the selection of cells producing the fusion protein. The resulting plasmid pCEP-mB7-H6 (ECD)-Fc (SEQ ID NO:23) drove the expression of a mB7-H6 (ECD)-Fc domain fusion protein under the control of a CMV promoter.

The pCEP-comp-FL-C was identical to pCEP-SP-Xa1-Fc* except that the nucleic acid sequence encoding for SP-Xa1-Fc* part was replaced by nucleic acid sequences encoding for the rat comp pentamerization domain fused with a C-terminal FLAG tag. The resulting plasmid pCEP-mB7-H6 (ECD)-comp-FL-C (SEQ ID NO:24) drove the expression of a mB7-H6 (ECD) fused to “comp” pentamerizaion domain containing FLAG (FL) tag at the C terminus under the control of a CMV promoter.

Expression and purification of the mB7-H6 (ECD)-Fc domain and the mB7-H6 (ECD)-comp-Flag domain fusion protein protein were performed according detailed descriptions in example 3.

### EXAMPLE 10

Molecular Cloning of the Human B7-H6

For the cDNA synthesis 4 μg human spleen polyA⁺ RNA (Cat No. 6542-1, Clontech Laboratories, Inc.) was used. The 1^{st} strand cDNA was synthesized in a reaction containing 50 mM Tris-HCl, pH 8.3, 75 mM KCl, 3 mM MgCl₂, 10 mM dithiothreitol, 500 μM dATP, dCTP, dGTP, dTTP, 25 μg/ml oligo(dT)12-18, 40 Units RNaseOUT (Invitrogen life technologies, Cat. No. 10777-019), and 200 Units SUPERSCRIPT™ II RNase H⁻ reverse transcriptase (Invitrogen life technologies, Cat. No. 18064-022) in a total volume of 20 μl at 42° C. for 1 hour. Following the reverse transcription the reaction was terminated by incubation at 85° C. for 5 minutes. To remove the complementary RNA prior to PCR the cDNA was treated with 2 units of RNase H at 37° C. for 30 minutes.

The cDNA sequence of human B7-H6 containing the complete open reading frame was amplified by PCR. The PCR was performed using spleen derived cDNA as template as well as the High Fidelity PCR System composed of a unique enzyme mix containing thermostable Taq DNA polymerase, a proofreading polymerase (Roche, Cat. No. 1 732 650), and the primers B76-1 (5′-AGG AGG CTG GAA GAA AGG AC-3′) (SEQ ID NO: 47) and B76-2 (5′-CCC CCG GCA GAG ATA CTA-3′) (SEQ ID NO: 48). The PCR product (1466 base pairs) of hsB7-H6 obtained from the mouse spleen cDNA was cloned into pCR II Topo plasmid using T4 DNA ligase (Promega, Cat. No. A3600). After ligation the plasmid was used to transform competent E. coli strain XL1-Blue. The nucleic acid sequence of hsB7-H6 (SEQ ID NO: 41) was verified by DNA sequencing of four independent clones.

### EXAMPLE 11

Preparation and Purification of Soluble (Secreted) Form of Human B7-H6 Protein

Production of Soluble hsB7-H6

In order to produce large amount of soluble mB7-H6 protein, a plasmid encoding a secreted form of hsB7-H6 fused to the Fc constant region of human IgG1 or the FLAG tagged rat comp pentamerisation domain was introduced into eukaryotic cell and hsB7-H6 expressing cells were selected using geneticin.

In more detail, a DNA fragment encoding a secreted form of hsB7-H6, designated hsB7-H6 (ECD) (SEQ ID NO: 43), was constructed by polymerase chain reaction (PCR) as follow: The full length hsB7-H6 cDNA clone (described in example 19) was used as template. The PCR reaction was performed using the High Fidelity PCR System composed of a unique enzyme mix containing termostable Taq DNA polymerase and a proofreading polymerase (Roche, Cat. No. 1 732 650), and 10 picomoles each of a sense and an antisense oligonucleotide primer in a final volume of 50 microliters. The sense oligonucleotide primer, designated B76-3, had the sequence 5′-GGT ACC GCC ACC ATG GGG ATC TTA CTG GGC CT-3′ (SEQ ID NO: 49) and contained the recognition site for the restriction enzyme KpnI (GGTACC), the strong translation initiation site (GCCACCATGG) and was identical to the human hsB7-H6 cDNA from nucleotides 6 to 25 (SEQ ID NO: 41). The antisense designated B76-4 had the sequence 5′-GCT AGC TTT CCT GGC CCA GCA CT-3′ (SEQ ID NO: 50) and contained the recognition site for the restriction enzyme NheI (GCTAGC) to fuse to the Fc constant region of human IgG1 or comp-FLAG domain and is identical, in an antisense orientation, to the hsB7-H6 cDNA from nucleotides 828 to 845 (SEQ ID NO: 41).

The PCR reaction was performed on a Hybaid programmable thermal cycler with 5 cycles of 94°, 30 sec, 50°, 45 sec, 72°, 60 sec, and 25 cycles of 94°, 30 sec, 72°, 70 sec and a final cycle of 72°, 7 min. The resulting PCR product which extended from hsB7-H6 nucleotide 6-845 was flanked by restriction sites. In the cell, this DNA encodes a secreted form of the hsB7-H6 protein from methionine amino acid 1 to lysine amino acid 280 (SEQ ID NO: 42). The PCR product was confirmed by sequencing.

The DNA was digested with KpnI and NheI and the insert, containing the nucleic acid molecule encoding for the extracellular domain (ECD) of hsB7-H6 (SEQ ID NO: 43), was ligated into each pCEP-SP-Xa1-Fc* and pCEP-comp-FL-C expression vector. Both vectors were derivatives of the episomal mammalian expression vector pCEP4 (Invitrogen), carrying the Epstein-Barr Virus replication origin (oriP) and nuclear antigen (encoded by the EBNA-1 gene) to permit extrachromosomal replication, and contained a Puromycin selection marker in place of the original Hygromycin B resistance gene.

The pCEP-SP-Xa1-Fc* is an expression vector that contains a KpnI cloning site downstream of the strong cytomegalo virus (CMV) promoter, a NheI cloning site upstream of the Factor X protease recognition site flanking the N-terminus of the Fc constant region of the human IgG1 and a SV40 poly(A) signal necessary for expression in mammalian cells. In addition, the vector contains the EBNA, origin of replication, ampilicin resistance gene, puromycin resistance gene for the selection of cells producing the fusion protein. The resulting plasmid pCEP-hsB7-H6 (ECD)-Xa1-Fc* (SEQ ID NO: 46) drove the expression of a hsB7-H6 (ECD)-Fc domain fusion protein under the control of a CMV promoter.

The pCEP-comp-FL-C was identical to pCEP-SP-Xa1-Fc* except that the nucleic acid sequence encoding for SP-Xa1-Fc* part was replaced by nucleic acid sequences encoding for the rat comp pentamerization domain fused with a C-terminal FLAG tag. The resulting plasmid pCEP-hsB7-H6 (ECD)-comp-FL-C (SEQ ID NO: 45) drove the expression of a hsB7-H6 (ECD) fused to “comp” pentamerizaion domain containing FLAG (FL) tag at the C terminus under the control of a CMV promoter.

Expression and purification of the hsB7-H6 (ECD)-Fc domain and the hsB7-H6 (ECD)-comp-Flag domain fusion protein protein were performed according detailed descriptions in example 3.

### EXAMPLE 12

Expression of hsB7-H4LV, hsB7-H5, mB7-H5, mB7-H6, hsB7-H6 mRNA.

The tissue distribution of the hsB7-H4LV mRNA was investigated by northern blot analysis and RT-PCR. For the northern blot radiolabeled RNA probes were used. The cDNA of human hsB7-H4LV, cloned into pGEM-T vector (described in example 3), and digested with KpnI restriction enzyme was used as template. KpnI restriction enzyme cuts 415 bp upstream of the stop codon. The in vitro synthesis of the RNA probe for hsB7-H4LV and human β-actin was performed according to the protocol of the instruction manual (Strip-EZ™ RNA SP6 Kit, Ambion; Cat No 1360BI) using SP6 polymerase. Free nucleotides were removed from radiolabeled DNA probes using Microspin G-25 columns (Amersham Pharmacia Biotech Inc.; Cat No 27-5226-01). Radiolabeled probes diluted in ULTRAhyb™ hybridization solution (Ambion; Cat No 8670) were added to the prehybridized blot and incubated 18 hours at 68° C. The hybridization buffer was discarded and the blot was washed twice 5 min in 2×SSC, 0.1% SDS at room temperature and then twice 15 min in 0.1×SSC, 0.1% SDS at 68° C. Northern blot was exposed to Kodak imaging for 1 week at −70° C. and developed using Agfa CP100.

Northern blot analysis using poly(A) enriched RNA from different adult human tissues revealed one hsB7-H4LV mRNA of approximately 3.8 kb. The highest level of hsB7-H4LV mRNA was observed in lung and a band of markedly lower intensity was found with RNA from thymus, kidney, skeletal muscle and placenta. Traces of hsB7-H4LV mRNA were detected in heart, pancreas, liver, and spleen, whereas no transcript was found in brain. To compare integrity and amount of RNA, a radiolabeled probe of β-actin was used for an identical northern blot. Similar conditions persisted for RNA derived from brain, placenta, heart, kidney, lung, spleen, and thymus. A rather low RNA amount was found in skeletal muscle, pancreas and liver.

For the RT-PCR analysis 0,5 ug of mRNA or 5 ug of total RNA of different tissues or cell lines were used as template for the cDNA synthesis. The cDNA synthesis was performed according to the protocol described in example 2 using SUPERSCRIPT™ II RNase H⁻ reverse transcriptase (Invitrogen life technologies, Cat. No. 18064-022). Alternatively Cytos in house pDEL libraries of different tissues and cell types were used as template.

The PCR for hsB7-H4LV was performed according to the protocol described in example 2. The highest amounts of specific PCR product were observed in testis, whereas low amounts were obtained from spleen. No PCR product was observed in brain.

The PCR for hsB7-H5 was performed according to the protocol described in example 4. The highest amounts of specific PCR product were observed in testis. No PCR product was observed in brain and spleen

The PCR for mB7-H5 was performed according to the protocol described in example 6. The highest amounts of specific PCR product were observed in lung, liver, brain, kidney, spinal cord, whereas lower amounts were obtained from naïve spleen, activated spleen, naïve dendritic cells, activated dendritic cells, lymphnodes, stomach, gut, ovaries and heart. No PCR product was observed in skeletal muscle, thymus, A20 cell line and C2C12 cell line.

The PCR for mB7-H6 was performed according to the protocol described in example 8. The highest amounts of specific PCR product were observed in activated dendritic cells, macrophages, lung and liver whereas lower amounts were obtained from naïve dendritic cells. No PCR product was observed from naïve B-cells, activated B-cells, T_{H}1-cells, T_{H}2-cells, EL-4 T-cell line, A20 cell line and C2C12 cell line.

The PCR for hsB7-H6 was performed according to the protocol described in example 10. A specific PCR product was obtained in human spleen.

### EXAMPLE 13

Stimulation of B Cell Proliferation but Not T Cell Proliferation by Mouse B7-H5

To investigate the role of mB7-H5 as a positive regulator of B cell activation a B cell proliferation assay was performed. In this assay purified B cells are stimulated by immobilized mB7-H5-Fc fusion protein in the presence or absence of immobilized anti-IgM antibody. Spleen from naïve mice were taken and passed through 70 μm Nylon cell strainer (Cat No. 352350; Falcon) to obtain splenocytes. The B cells were purified using the antibody against CD45R (B220) MACS beads system (Milteny Biotec, Auburn, Calif.). For proliferation assays, purified B cell (2×10⁵ cells/well in triplicate) were cultured in 96-well flat-bottom plates, that were pre-coated at 4° C. overnight with 75 μl/well with 0, 2.5, 5, 10 or 20 μg/μl of mB7-H5-Fc fusion protein (described in example 7) or mouse gamma globuline (Cat No. 015-000-002, Jackson ImmunoResearch Laboratories, Inc.) in the presence of 0, 0.25 or 0.5 μg/μl of goat anti mouse IgM (Fab′)2 (Cat No. 115-006-075; Jackson ImmunoResearch Laboratories, Inc.) diluted in PBS. For measurement of B cell proliferation, the plates were cultured for 60 to 72 h and [³H]-thymidine (1 μCi/well) was added 8 to 10 h prior to harvesting of the cultures. [³1H]-thymidine incorporation was measured with a MicroBeta Trilux Liquid Scintillation counter (Wallac, Turku, Finland). B cell proliferation was measured by [³H]-thymidine incorporation. Immobilized mB7-H5-Fc fusion protein resulted in a significantly higher B cell proliferation (FIG. 1A) compared to mouse gamma globuline (FIG. 1B). The positive regulatory effect of mB7-H5-Fc fusion protein on B cell proliferation is dose dependent and showed a co-stimulatory effect in combination with immobilized goat anti-mouse IgM antibody (FIG. 1A). These data indicate that mB7-H5 acts as positive regulator of B cell proliferation and shows co-stimulation in combination with other proliferative compounds, e.g. goat anti-mouse IgM. As mB7-H5 can induce B cell proliferation in an antigen independent manner, it may play an important role in the regulation of the B cell homeostasis. Note that B7-H5 did not influence T cell proliferation in vitro.

### EXAMPLE 14

B7-H6 Negatively Modulates T Cell Proliferation but Not B Cell Proliferation

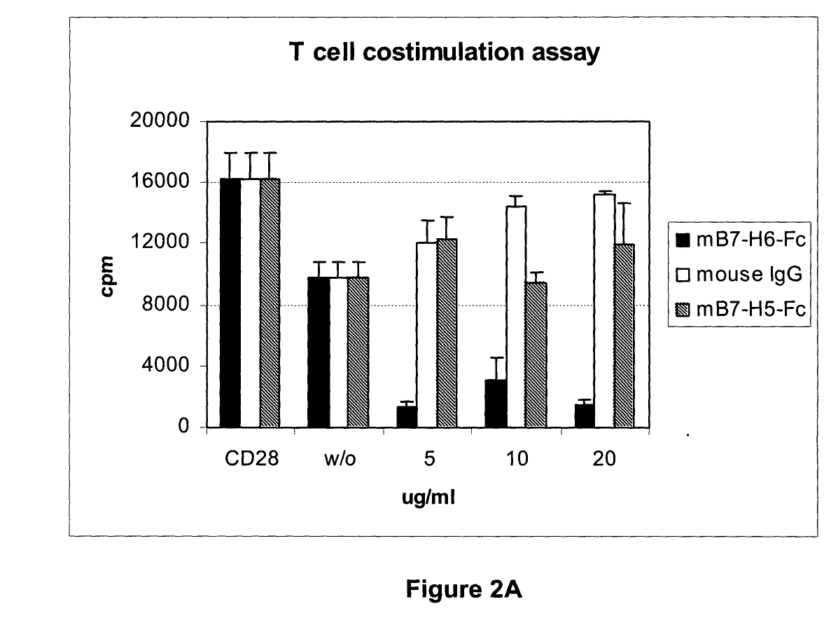

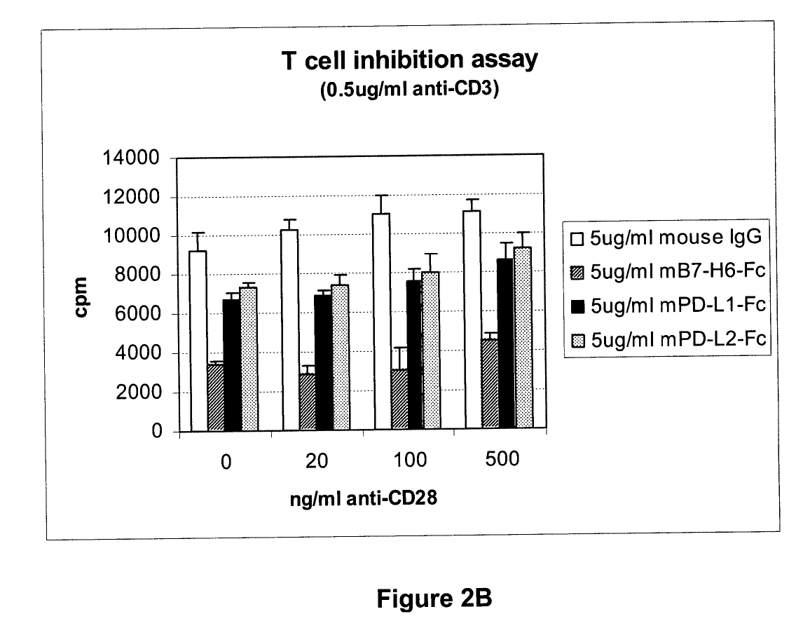

To investigate the role of mB7-H6 in T cell activation, a co-stimulation- and inhibition assays were performed. In these assays purified T cells were stimulated by immobilized anti-CD3 antibody in the presence of immobilized mB7-H6-Fc fusion protein. Spleen from naïve mice were taken and passed through 70 μm Nylon cell strainer (Cat No. 352350; Falcon) to obtain splenocytes. The T cells were purified using the antibody against CD4/8 MACS beads system (Milteny Biotec, Auburn, Calif.). For co-stimulation and inhibition assays, purified T cell (2×10⁵ cells/well in triplicate) were cultured in 96-well flat-bottom plates, that were pre-coated at 4° C. overnight with 75 μl/well with indicated concentration of mouse anti-CD3 epsilon chain antibody NA/LE (145-2C11; BD Bioscience, Pharmigen, San Diego, Calif.) in the presence of indicated concentrations of mB7-H6-Fc fusion protein (described in example 9) or control proteins, such as antibody against mouse CD28 NA/LE (37.51; BD Bioscience, Pharmigen, San Diego, Calif.), recombinant mouse B7-H1/Fc chimera (Cat No. 1019-B7; R&D Systems, Inc.), recombinant mouse PD-L2/Fc chimera (Cat No. 1022-PL; R&D Systems, Inc.) and mouse gamma globuline (Cat No. 015-000-002, Jackson ImmunoResearch Laboratories, Inc.). For measurement of T cell proliferation, the plates were cultured for 60 to 72 h and [³H]-thymidine (1 μCi/well) was added 8 to 10 h prior to harvesting of the cultures. [3H]-thymidine incorporation was measured with a MicroBeta Trilux Liquid Scintillation counter (Wallac, Turku, Finland). T cell proliferation was measured by [³H]-thymidine incorporation. In the co-stimulation assay, immobilized mB7-H6-Fc fusion protein resulted in a fivefold reduction of T cell proliferation compared to anti-CD3 antibody alone or plus mouse IgG and mB7-H5-Fc fusion protein (FIG. 2A). Anti-CD28 antibody as a positive control for T cell co-stimulation, showed a clear co-stimulatory effect. These data show that mB7-H6 can inhibit TCR mediated proliferation. T cells activated via T cell receptor plus CD28 using anti-CD3 and anti-CD28 antibodies show a threefold reduction in their proliferation in the presence of immobilized mB7-H6-Fc fusion protein compared to mouse IgG (FIG. 2B). The effect of PD-L1-Fc or PD-L2-Fc fusion proteins, two known negative regulators of T cell activation, was significantly less compared to mB7-H6-Fc. These results show that mB7-H6 is a strong negative regulatory of T cell activation. Note that B cell proliferation was not affected in vitro by B7-H6.

### EXAMPLE 15

Administration of mB7-H5-Fc Fusion Protein Affected the B Cells Homeostasis In Vivo

The mB7-H5-Fc fusion protein (example 7) was used to inject mice three times. The injection of the mB7-H5-Fc fusion protein resulted in a 5 times increase of isotype switched B cells (CD19+, IgD− & IgM−) compared with control mice obtained human IgG1 κ antibody and a twofold increase of total IgM and IgG serum levels.

The mice used in this experiment were 6-18 weeks old female C57B16. Groups of four mice were injected i.p. with 500 μg of mB7-H5-Fc fusion protein, or alternatively human IgG1κ (Cat No. 1-5154; Sigma-Aldrich Chemie Gmbh, Steinheim, Germany) on days −1, 1 and 3. At day 4 the mice were anesthetized by methoxyflurane inhalation and retrobulbar blood letting was performed to obtain serum for total IgM and IgG determinations. At day 10 the mice were anesthetized by methoxyflurane inhalation and retrobulbar blood letting was performed. The mice were sacrificed by cervical dislocation and spleen was dissected from each animal. Splenocytes were obtained by passing through 70 μm Nylon cell strainer (Cat No. 352350; Falcon). Three color staining of the splenocytes was perfomed to analyse the ratio of isotype switched B cells, naïve mature B cells and T cell, macrophages, granulocytes.

a) Detection of spleen-derived isotype switched B cells (CD19+, IgD− and IgM−), naïve mature B cells (CD19+, IgD+ and IgM+) and T cells, macrophages, granulocytes (CD4+, CD8+ and CD11b+) by a three colour staining using FACS. 2×10⁶ splenocytes from each mouse were used for the analysis. Fc receptors of splenocytes were blocked using rat anti-mouse CD16/CD32 (Fc gamma II/III receptor) monoclonal antibodies (Cat No. 01241A; BD Bioscience, Pharmigen, San Diego, Calif.). Splenocytes were washed and incubated 20 min. at 4° C. in an antibody solution mix containing rat anti-mouse CD19-PE monoclonal antibody (Cat No. 557399; BD Bioscience, Pharmigen, San Diego, Calif.), rat anti-mouse IgD-FITC monoclonal antibody (Cat No. 553439; BD Bioscience, Pharmigen, San Diego, Calif.), goat anti-mouse IgM-FITC 11 chain specific antibody (Cat No. 115-095-020; Jackson ImmunoResearch Laboratories, Inc.), rat anti-mouse CD8a-FITC (Ly-2) monoclonal antibody (Cat No. 553031; BD Bioscience, Pharmigen, San Diego, Calif.), rat anti-mouse CD4-FITC (L3T4) monoclonal antibody (Cat No. 557307; BD Bioscience, Pharmigen, San Diego, Calif.) and rat anti-mouse CD11b-FITC monoclonal antibody (Cat No. 553310; BD Bioscience, Pharmigen, San Diego, Calif.). Splenocytes were washed, resuspended in FACS buffer (2% FCS, 0.05% NaN3 in PBS) containing 1 μg/ml PI and analysed. For the groups of mB7-H5-Fc the percentage of isotype switched B-cells (CD19+, IgD− and IgM−) was fivefold increased compared to control and naïve mice, respectively (FIG. 3A). On the other hand the percentage of naïve mature B cells (CD19+, IgD+ and IgM+) were significantly reduced (p <0.02) (FIG. 3B) and the percentage of T cell, macrophages, and granulocytes were increased. These observations were in accordance with the positive regulatory effect on B cell proliferation (example 13). However it is not clear if mB7-H5 play a role in the differentiation of B cells and/or in the division of B cells. In summary B7-H5 might play an important role in the regulation of B cell homeostasis. This observation is insofar surprising as the B and T lymphocytes are produced continuously either in the primary lymphoid organs or by peripheral cell division, however the total number of T and B cells remain constant. The mechanisms that determine the number of peripheral lymphocytes are poorly understood mB7-H5 might be the first member of a novel family regulating the B cell homeostasis in mice.

b) Measurement of total IgM and IgG serum levels at day 4 and 10 of the different experimental groups. For the measurement 96-well F96 MaxiSorp Nunc-Immunoplates (Cat No. 442404; Nalge Nunc International), that were pre-coated at 4° C. overnight with serum of each mice, diluted 1:600 in 0.1 M NaHCO₃ pH 9.6 (in triplicates) were used. Plates were washed four times with PBS-Tween20 and background was reduced by incubating plates 2 h at 37° C. in blocking buffer (2% BSA (Cat No. A-3803; Sigma) in PBS-Tween20). Plates were washed five times and 1:1000 diluted detection antibody (anti mouse IgM HRPO-coupled (Cat No. A8786; Sigma) and anti mouse IgG HRPO coupled (Cat No. A3673; Sigma), respectively) was incubated for 1 h at room temperature. Plated were washed five times with PBS-Tween20 and detection was performed using OPD substrate solution (0.066 M Na₂HPO₄, 0.035 M citric acid pH5.0 containing 10 mg OPD (Cat No. 78446; Fluka) and 8 μl of 30% H₂O₂ (Cat No. 95302; Fluka) per 25 ml) and 5% H₂SO₄ in H₂O as stop solution. The absorbance was measured using ELISA reader (BioRad Benchmark) at 450 ηm and for calculation of arithmetic means and standard error of the mean (SEM) deviation EXCEL software (MS Office; Microsoft) was used. The serum levels of total IgM and IgG are at least twofold increased for the group of mice obtained mB7-H5-Fc fusion protein compared to the group obtained a control protein or to naïve mice (Table 2). Except at day 4 the total IgG serum levels are for all three groups the same. However this is in accordance to the fact the IgG response is following the IgM response and appears at later time points. This data is in accordance with the positive regulatory effect of mB7-H5-Fc on B cell proliferation observed in vitro. Thus mB7-H5 might be a novel member of a molecule family which is involved in the regulation of the B cell homeostasis.

TABLE 2
Average of total IgM or IgG serum levels
Absorption (OD450 ηm)

| | Total IgM | | Total IgG | |
|---|---|---|---|---|
| Experimental group | Day 4 | Day 10 | Day 4 | Day 10 |
| Control | 0.148 ± 0.001 | 0.156 ± 0.007 | 0.335 ± 0.017 | 0.317 ± 0.014 |
| mB7-H5-Fc | 0.278 ± 0.009 | 0.363 ± 0.014 | 0.414 ± 0.005 | 0.680 ± 0.007 |
| Naïve | 0.157 ± 0.023 | 0.131 ± 0.023 | 0.416 ± 0.001 | 0.319 ± 0.010 |

### EXAMPLE 16

Administration of mB7-H5-Fc Fusion Protein and Additional Qβ Immunization Modulated Qβ Specific B Cell In Vivo

The mB7-H5-Fc fusion protein (example 7) was used to inject mice three times. The injection of the mB7-H5-Fc fusion protein and additional Qβ immunization resulted in a twofold increase of isotype switched B cells (CD19+, IgD− & IgM−) and total IgM and IgG serum levels compared to control mice. In contrast the Qβ-specific humoral immune response was reduced at least twofold mB7-H5 injection affected T cell independent IgM responses similarly as T cell dependent IgG responses. This suggests that mB7-H5 directly acts on B cells (Bachmann M. F and Kundig T. M. (1994) Curr. Opin. Immunol. 6, 320-6), which is consistent with the in vitro results (Example 13)

The mice used in this experiment were 6-18 weeks old female C57B16. Groups of five mice were injected i.p. 500 μg of mB7-H5-Fc fusion protein, or alternatively mouse adiponectin-Fc fusion protein (Acrp16-Fc) on days −1, 1 and 3. On day 0 an additional injection of 50 μg wildtype Qβ s.c. was done. At day 10 the mice were anesthetized by methoxyflurane inhalation and retrobulbar blood letting was performed. The mice were sacrificed by cervical dislocation and spleen was dissected from each animal. Splenocytes were obtained by passing through 70 μm Nylon cell strainer (Cat No. 352350; Falcon). Four color staining of the splenocytes was perfomed to analyse the ratio of Qβ-specific B cells, isotype switched B cells, naïve mature B cells and T cell, macrophages, granulocytes. Further an antibody-forming cell assay (AFC) and ELISA specific for Qβ were performed.

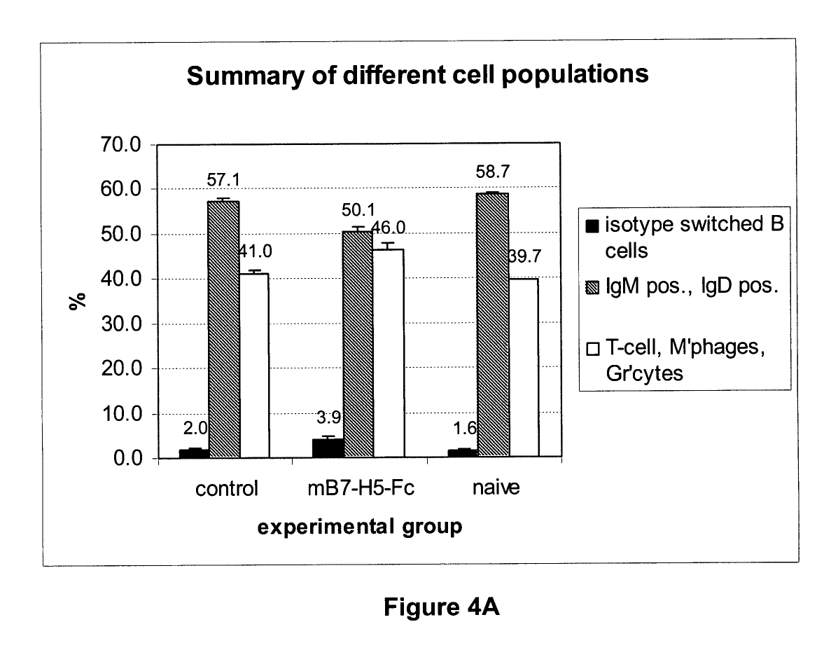

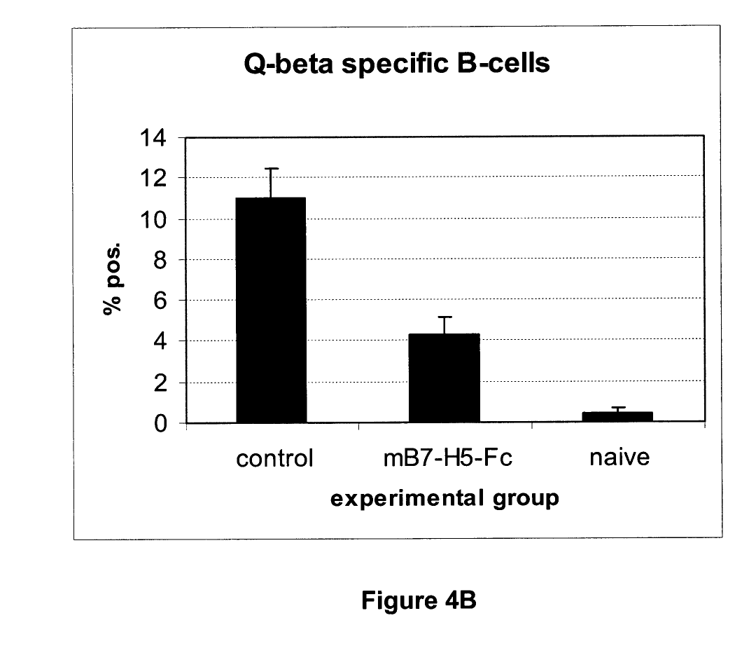

a) Detection of spleen-derived Qβ-specfic B cells, isotype switched B cells (CD19+, IgD− and IgM−), naïve mature B cells (CD19+, IgD+ and IgM+) and T cells, macrophages, granulocytes (CD4+, CD8+ and CD11b+) by a four colour staining using FACS. 2×10⁶ splenocytes from each mouse were used for the analysis. Splenocytes were resuspended with 3 μg/ml wildtype Qβ in FACS buffer (2% FCS, 0.05% NaN3 in PBS) and incubated 30 min at 4° C. Fc receptors of splenocytes were blocked using rat anti-mouse CD16/CD32 (Fc gamma II/III receptor) monoclonal antibodies (Cat No. 01241A; BD Bioscience, Pharmigen, San Diego, Calif.). Splenocytes were washed, resuspended in rabbit anti-Qβ serum ditluted 1:400 in FACS buffer and incubated 30 min at 4° C. After two washing steps the splenocytes were resuspended in an antibody solution mix containing rat anti-mouse CD19-PE monoclonal antibody (Cat No. 557399; BD Bioscience, Pharmigen, San Diego, Calif.), rat anti-mouse IgD-FITC monoclonal antibody (Cat No. 553439; BD Bioscience, Pharmigen, San Diego, Calif.), goat anti-mouse IgM-FITC μ chain specific antibody (Cat No. 115-095-020; Jackson ImmunoResearch Laboratories, Inc.), rat anti-mouse CD8a-FITC (Ly-2) monoclonal antibody (Cat No. 553031; BD Bioscience, Pharmigen, San Diego, Calif.), rat anti-mouse CD4-FITC (L3T4) monoclonal antibody (Cat No. 557307; BD Bioscience, Pharmigen, San Diego, Calif.) and rat anti-mouse CD11b-FITC monoclonal antibody (Cat No. 553310; BD Bioscience, Pharmigen, San Diego, Calif.) and incubated for 20 min at 4° C. Splenocytes were washed, resuspended in FACS buffer containing 1 μg/ml PI and analysed. For the groups of mB7-H5-Fc the percentage of isotype switched B-cells (CD19+, IgD− and IgM−) was increased at least twofold compared to control and naïve mice respectively (FIG. 4A). Further the naïve mature B cells (CD19+, IgD+ and IgM+) were significantly reduced (p<0.02) (FIG. 4A). On the other hand the Qβ-specific B cells were depleted by at least twofold (FIG. 4B). These results were consistently with the observation that InB7-H5 is an upregulator of B-cell proliferation in vitro, made in example 15.

b) mB7-H5-Fc administration reduced the number of Qβ-specific antibody-forming cells. 24-well plates were pre-coated with 25 μg/ml wildtype Qβ in 0.1 M NaHCO₃ pH 9.6 overnight at 4° C. and blocked for 2 h at room temperature using 2% BSA (Cat No. A3803, Sigma) in PBS. Plates were washed three times with PBS-Tween20 and once with cell culture medium. The splenocytes were resuspended to 5×10⁶ cells/ml and plated in dilution serie 1:5 per well. Following 5 h incubation at 37° C. the plates were washed five times with PBS-Tween20 and incubated with goat anti-mouse IgG antibody (Cat No. AT-2306-2; EY Laboratories) diluted 1:1000 in 2% BSA/PBS overnight at room temperature. After washing the plates were incubated with donkey anti-goat IgG-AP coupled (Cat No. 705-055-147; Jackson ImmunoResearch Laboratories, Inc.) 3 h at 37° C. For the color reaction 1 ml/well of substrate solution containing 4 parts of alkaline buffer solution (Cat No Sigma Diagnostic Inc., St Louis, USA) containing 1 mg/ml BCIP 5-Bromo-4-chloro-3-indolylphosphate p-toluidine salt (Cat No. 16670; Fluka BioChemika) and 1 part 3% Agarose in H₂O. Dots were counted and normalized to 10⁶ cells per well. For calculation of arithmetic means and standard error of the mean (SEM) EXCEL software (MS Office; Microsoft) was used. The Qβ specific antibody-forming cells were decreased at least by a factor of three in the group of mice obtained mB7-H5-Fc fusion protein compared to the control group (Table 3). This result is in accordance with the reduction of Qβ specific B cells described in example 16a. The Qβ specific B cell detected using AFC assay reflecting B cell secreting specific antibodies such as plasma cells. On the other hand Qβ specific B cell detected via flow cytometry as in example 16a reflecting B memory cells. The data indicated a clear reduction of the humoral immune response.

TABLE 3
Qβ specific antibody forming cells
Dots per 10⁶ cells

| Experimental | Arithmetic | |
|---|---|---|
| group | mean | SEM |
| Control | 133 | 14 |
| mB7-H5-Fc | 37 | 5 |
| Naïve | 0 | 0 |

c) Measurement of Qβ specific IgM and IgG antibody titers in serum at day 10. For the measurement 96-well F96 MaxiSorp Nunc-Immunoplates (Cat No. 442404; Nalge Nunc International), that were pre-coated at 4° C. overnight with 3 μg/ml wildtype Qβ (batch Qx 2.2; Cytos Biotechnology AG, Schlieren) in 0.1 M NaHCO₃ pH 9.6 were used. Plates were washed four times with PBS-Tween20 and background was reduced by incubating plates 2 h at 37° C. in blocking buffer (2% BSA (Cat No. A-3803; Sigma) in PBS-Tween20). The serum was diluted in serum dilution buffer (2% BSA, 1% FCS in PBS-Tween20. Every sample was analyzed in duplicates and lowest serum dilution was 1:40. Twofold dilution steps were done and incubated for 2 h at room temperature on ELISA plate shaker (Heidolph Titramax 100). Plates were washed five times and 1:1000 diluted detection antibody (anti mouse IgM HRPO-coupled (Cat No. A8786; Sigma) and anti mouse IgG HRPO coupled (Cat No. A3673; Sigma), respectively) was incubated for 1 h at room temperature. Plated were washed five times with PBS-Tween20 and detection was performed using OPD substrate solution (0.066 M Na₂HPO₄, 0.035 M citric acid pH5.0 containing 10 mg OPD (Cat No. 78446; Fluka) and 8 μl of 30% H₂O₂ (Cat No. 95302; Fluka) per 25 ml) and 5% H₂SO₄ in H₂O as stop solution. The absorbance was measured using ELISA reader (BioRad Benchmark) at 450 μm and for calculation of arithmetic means and standard error of the mean (SEM) EXCEL software (MS Office; Microsoft) was used. The Qβ specific IgM and IgG antibody titers were threefold reduced for the group, that obtained mB7-H5-Fc compared with the control group (Table 4). This result was in accordance with the reduction of Qβ specific antibody forming cells observed in Example 16b. Note that IgM and IgG titers are similarly affected, indicating that mB7-H5 acts directly on B cells

TABLE 4
Qβ specific IgM and IgG antibody titers at day 10
Serum dilution giving half maximal Absorption
(OD450 ηm)

| Experimental | | |
|---|---|---|
| group | IgM | IgG |
| Control | 1452 ± 56 | 1932 ± 114 |
| mB7-H5-Fc | 482 ± 28 | 711 ± 118 |
| Naïve | 116 ± 18 | 0 ± 0 |

d) Measurement of total IgM and IgG serum levels at day 10 in the different experimental groups. For the measurement 96-well F96 MaxiSorp Nunc-Immunoplates (Cat No. 442404; Nalge Nunc International), that were pre-coated at 4° C. overnight with serum of each mice, diluted 1:600 in 0.1 M NaHCO₃ pH 9.6 (in triplicates) were used. Plates were washed four times with PBS-Tween20 and background was reduced by incubating plates 2 h at 37° C. in blocking buffer (2% BSA (Cat No. A-3803; Sigma) in PBS-Tween20). Plates were washed five times and 1:1000 diluted detection antibody (anti mouse IgM HRPO-coupled (Cat No. A8786; Sigma) and anti mouse IgG HRPO coupled (Cat No. A3673; Sigma), respectively) was incubated for 1 h at room temperature. Plated were washed five times with PBS-Tween20 and detection was performed using OPD substrate solution (0.066 M Na₂HPO₄, 0.035 M citric acid pH5.0 containing 10 mg OPD (Cat No. 78446; Fluka) and 8 μl of 30% H₂O₂ (Cat No. 95302; Fluka) per 25 ml) and 5% H₂SO₄ in H₂O as stop solution. The absorbance was measured using ELISA reader (BioRad Benchmark) at 450 ηm and for calculation of arithmetic means and standard error of mean (SEM) EXCEL software (MS Office; Microsoft) was used. The serum levels of total IgM and IgG were twofold increased for the group that obtained mB7-H5-Fc fusion protein compared to control group or naïve mice (Table 5).

TABLE 5
Total IgM and IgG serum levels at day 10
Absorption (OD450 ηm)

| Experimental | | |
|---|---|---|
| group | Total IgM | Total IgG |
| Control | 0.189 ± 0.014 | 0.342 ± 0.030 |
| mB7-H5-Fc | 0.320 ± 0.020 | 0.630 ± 0.021 |
| Naïve | 0.120 ± 0.003 | 0.330 ± 0.022 |

Thus the administration of mB7-H5-Fc fusion protein leaded to shift in the balance of the numbers of different lymphocytes. The reduced Qβ specific immune response observed in the different assays might be a secondary effect, which is the consequence of an increased number of isotype switched B cells. The mechanisms which regulate the total number of T and B cells are poorly understood. In summary mB7-H5 may act as a regulator of B cell homeostasis and modulator of the specific B cell response

### EXAMPLE 17

Administration of mB7-H6-Fc Fusion Protein and Additional Qβp33xNKpt Immunization in Mice: In Vivo Reduction of T Cell Responses

The mB7-H6-Fc fusion protein (example 9) was used to inject mice three times. The injection of the mB7-H6-Fc fusion protein and additional Qβp33xNKpt immunization resulted in a reduction of the immune response compared to control mice. The mice used in this experiment were 6-18 weeks old female C57B16. Groups of three mice were injected i.p. 500 μg of mB7-H6-Fc fusion protein, or alternatively human IgG1κ (Cat No. I-5154; Sigma-Aldrich Chemie Gmbh, Steinheim, Germany) on days −1, 1 and 3. On day 0 an additional injection of 50 μg Qβp33xNKpt (short form) s.c. was done. At day 4 the mice were anesthetized by methoxyflurane inhalation and retrobulbar blood letting was performed to obtain serum for Qβ specific antibody and total IgM and IgG antibody level determinations. At day 10 the mice were anesthetized by methoxyflurane inhalation and retrobulbar blood letting was performed. The mice were sacrificed by cervical dislocation and spleen was dissected from each animal. Splenocytes were obtained by passing through 70 μm Nylon cell strainer (Cat No. 352350; Falcon). Four color staining of the splenocytes was perfomed to analyse the ratio of Qβ-specific B cells, isotype switched B cells, naïve mature B cells and T cell, macrophages, granulocytes. Further a Qβ specific antibody-forming cell assay and ELISA were performed. To monitor the T cell response a Gp33-H2-D^{b}-tetramer staining of blood lymphocytes and an intracellular interferon-γ staining of in vitro Qβ or p33 stimulated T cells were performed.

a) To investigate the role of mB7-H6 in the modulation of the CTL response, 3 drops of fresh blood was mixed in FACS buffer (2% FCS in PBS, 5 mM EDTA, pH 8.0) to detect p33 specific T cells by FACS analysis. The lymphocytes were incubated in Gp33-H2-D^{b}-tetramer-PE for 10 min. at room temperature. Rat anti-mouse CD8a (Ly2)-APC monoclonal antibody (Cat No. 553035; BD Bioscience, Pharmigen, San Diego, Calif.) was added and the incubation was prolonged for 30 min at 4° C. The lymphocytes were washed in FACS buffer and resuspended in 10% FACS™ Lysing solution (Cat No. 349202; BD Bioscience, California). The lymphocytes were washed and resuspended in FACS buffer for FACS analysis. For the group obtained mB7-H6-Fc fusion protein a twofold reduction of the p33 specific T cells was observed compared to control group (Table 6). This data was consistent with the negative regulation of T cell activation observed in vitro (FIGS. 2A and 2B). The reduction of the p33 specific T cells may be explained by the downregulation of the T cell response after mB7-H6-Fc fusion protein administration.

TABLE 6
Percentage of p33 specific T cells

| Experimental group | Average % gated (± SEM) |
|---|---|
| Control | 6.19 ± 1.62 |
| mB7-H6-Fc | 3.66 ± 1.13 |
| Naïve | 0.16 |

b) To investigate the role of mB7-H6 in the modulation of the T_{H} response, 2.5 10⁶ splenocytes from immunized mice were added to 96 well flat bottom plates and placed on ice. Anti CD11c MACS beads systems (Milteny Biotec, Auburn, Calif.) purified mouse dendritic cells (DC) were pulsed either with 20 μg/ml Qβ or 2 μM p33 peptide for 2 h at 37° C. Pulsed DCs were added to the splenocytes and incubated for 2 h at 37° C. 2.5 μg/well BrefeldinA was added and incubation prolonged for 6h. The cell were resuspended in FACS buffer (2% FCS, 0.05% NaN₃ in PBS) and incubated in rat anti-mouse CD8-FITC monoclonal antibody (Cat No. 553031; BD Bioscience, Pharmigen, San Diego, Calif.) for 20 min on ice. Cells were washed with FACS buffer and resuspended in 4% formalin in PBS. The fixed cell were washed, resuspended with rat anti-mouse Interferon-γ-APC monoclonal antibody (Cat No. 554413; BD Bioscience, Pharmigen, San Diego, Calif.) in 0.5% saponin, FACS buffer and incubated for 30 min. at room temperature. The cells were washed and FACS analysis was performed. For the group of mB7-H6-Fc fusion protein a reduction of the percentage of Interferon-γ producing CD8 positive T cells was observed compared with control mice (Table 7). Thus mB7-H6 induced a downregulation of the T_{H} response in vivo.

TABLE 7
Intracellular Interfon-γ

| Experimental | | % of CD8/p33 |
|---|---|---|
| group | % of CD8/Qβ (± SEM) | (± SEM) |
| Control | 0.41 ± 0.14 | 0.45 ± 0.12 |
| mB7-H6-Fc | 0.25 ± 0.08 | 0.31 ± 0.12 |
| naive | 0.11 | 0.14 |

Qβ induces T_{H} cell independent IgM antibodies followed by T_{H} cell dependent IgG responses. Thus, reduced IgM responses upon immunization with Qβ reflect impaired B cell responses while reduced IgG responses along with normal IgM responses indicates reduced T helper cell (Bachmann M. F and Kundig T. M. (1994) Curr. Opin. Immunol. 6, 320-6).

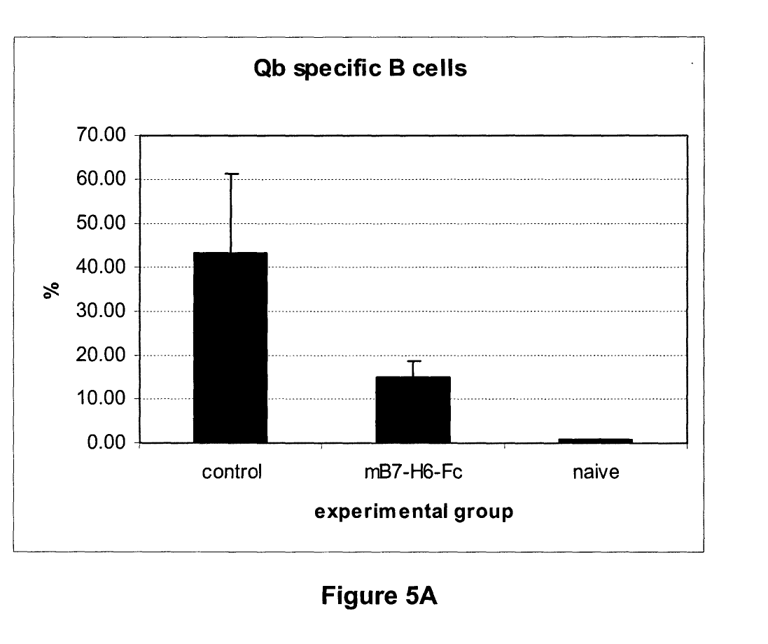

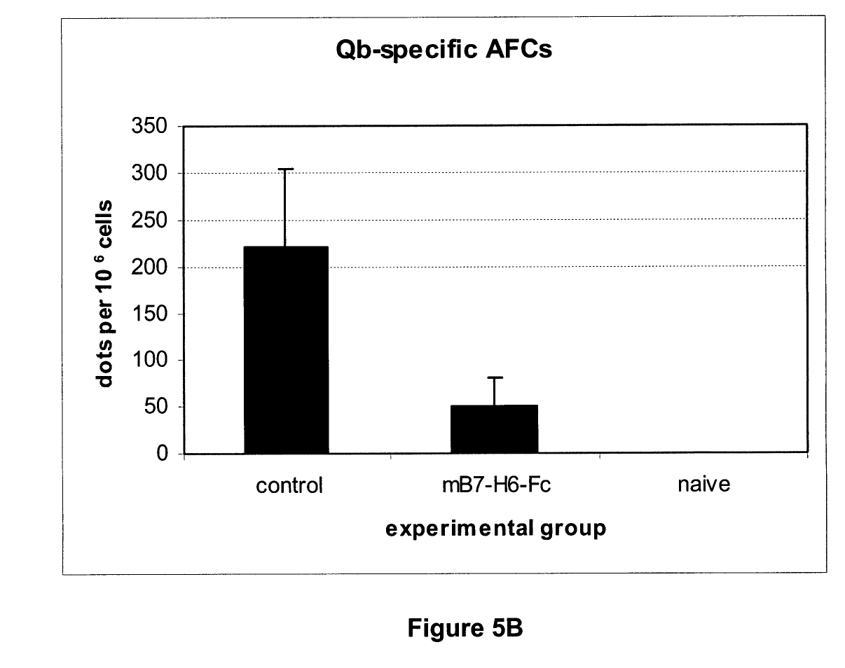

c) Detection of spleen-derived Qβ-specfic B cells, isotype switched B cells (CD19+, IgD− and IgM−), naïve mature B cells (CD19+, IgD+ and IgM+) and T cells, macrophages, granulocytes (CD4+, CD8+ and CD11b+) by a four colour staining using FACS. 2×10⁶ splenocytes from each mouse were used for the analysis. Splenocytes were resuspended with 3 μg/ml Qβ in FACS buffer (2% FCS, 0.05% NaN3 in PBS) and incubated 30 min at 4° C. Fc receptors of splenocytes were blocked using rat anti-mouse CD16/CD32 (Fc gamma II/III receptor) monoclonal antibodies (Cat No. 01241A; BD Bioscience, Pharmigen, San Diego, Calif.). Splenocytes were washed, resuspended in rabbit anti-Qβ serum ditluted 1:400 in FACS buffer and incubated 30 min at 4° C. After two washing steps the splenocytes were resuspended in an antibody solution mix containing rat anti-mouse CD19-PE monoclonal antibody (Cat No. 557399; BD Bioscience, Pharmigen, San Diego, Calif.), rat anti-mouse IgD-FITC monoclonal antibody (Cat No. 553439; BD Bioscience, Pharmigen, San Diego, Calif.), goat anti-mouse IgM-FITC p chain specific antibody (Cat No. 115-095-020; Jackson ImmunoResearch Laboratories, Inc.), rat anti-mouse CD8a-FITC (Ly-2) monoclonal antibody (Cat No. 553031; BD Bioscience, Pharmigen, San Diego, Calif.), rat anti-mouse CD4-FITC (L3T4) monoclonal antibody (Cat No. 557307; BD Bioscience, Pharmigen, San Diego, Calif.) and rat anti-mouse CD11b-FITC monoclonal antibody (Cat No. 553310; BD Bioscience, Pharmigen, San Diego, Calif.) and incubated for 20 min at 4° C. Splenocytes were washed, resuspended in FACS buffer containing 1 μg/ml PI and analysed. For the groups of mB7-H6-Fc the percentage of isotype switched B-cells (CD19+, IgD− and IgM−) was slightly reduced compared to control. The number of naïve mature B cells (CD19+, IgD+ and IgM+) and the T cells, macrophages and granulocytes remained unaffected. On the other hand the Qβ-specific B cells of the mice, that obtained mB7-H6-Fc fusion protein, were threefold reduced compared to the control mice (FIG. 5A). The lymphocytes homeostasis was not significantly altered by the administration of mB7-H6-Fc fusion protein, and control protein. In comparison the administration of mB7-H5-Fc fusion protein induced a shift in the lymphocyte homeostasis (see example 15 and 16). Therefore this reduction of the percentage of Qβ-specific B cells can not be explained by an increase of isotype switched B cells. In fact, the inhibitory effect of mB7-H6 on T cell activation most likely contribute to this reduction of Qβ-specific B cells.

d) In order to study the role of mB7-H6 on antibody secreting cells, a Qβ-specific IgG antibody forming cell assay (AFC) was performed mB7-H6-Fc administration reduced the number of isotype switched Qβ-specific antibody-forming cells. 24-well plates were pre-coated with 25 μg/ml Qβ in 0.1 M NaHCO₃ pH 9.6 overnight at 4° C. and blocked for 2 h at room temperature using 2% BSA (Cat No. A3803, Sigma) in PBS. Plates were washed three times with PBS-Tween20 and once with cell culture medium. The splenocytes were resuspended to 5×10⁶ cells/ml and plated in dilution serie 1:5 per well. Following 5 h incubation at 37° C. the plates were washed five times with PBS-Tween20 and incubated with goat anti-mouse IgG antibody (Cat No. AT-2306-2; EY Laboratories) diluted 1:1000 in 2% BSA/PBS overnight at room temperature. After washing the plates were incubated with donkey anti-goat IgG-AP coupled (Cat No. 705-055-147; Jackson ImmunoResearch Laboratories, Inc.) 3 h at 37° C. For the color reaction 1 ml/well of substrate solution containing 4 parts of alkaline buffer solution (Cat No.221; Sigma Diagnostic Inc., St Louis, USA) containing 1 mg/ml BCIP 5-Bromo-4-chloro-3-indolylphosphate p-toluidine salt (Cat No. 16670; Fluka BioChemika) and 1 part 3% Agarose in H₂O. Dots were counted and normalized to 10⁶ cells per well. For calculation of arithmetic means and standard deviation EXCEL software (MS Office; Microsoft) was used. The Qβ specific antibody-forming cells were decreased fourfold in the group of mice, that obtained mB7-H6-Fc fusion protein compared to the control mice (FIG. 5B). This result was in agreement with the observation made for Qβ specific B cells (see example 17c, FIG. 5A) and in fact also confirmed the reduction T_{H} response (Example 17b)

e) Since the Qβ specific B memory cells (example 17c) and plasma cells (example 17d) showed a significant reduction for the group that obtained mB7-H6-Fc fusion protein compared to control group Qβ specific IgM and IgG antibody titers in serum at day 4 and 10 were measured. The assay was performed according to detailed description in example 16c. Qβ specific IgM and IgG antibody titers at day 10 were about threefold reduced for the group, that obtained mB7-H6-Fc compared with the control group (Table 8). In contrast the Qβ specific IgM antibody titer at day 4 was only marginally reduced. Thus mB7-H6 plays a role as a negative regulator of the T_{H} cell dependent Ig response in vivo. Thus, normal IgM responses along with reduced IgG responses indicate reduced T help. These results were congruent with the observation, that mB7-H6 acts as a negative modulator of T cell activation in vitro (see Example 14).

TABLE 8
Qβ specific IgM and IgG antibody titers
Serum dilution giving half maximal Absorption (OD450 ηm)

| | |
|---|---|
| IgM | IgG |

| Experimental group | Day 4 | Day 4 | Day 10 |
|---|---|---|---|
| Control | 676 ± 87 | 158 ± 7 | 4250 ± 539 |
| mB7-H6-Fc | 461 ± 27 | 151 ± 2 | 1515 ± 157 |
| Naïve | 99 ± 31 | 156 ± 11 | 339 ± 334 |

f) Measurement of total IgM and IgG serum levels at day 4 and 10 in different experimental groups. The assay was performed according to detailed description in example 15a. No significant difference was observerd for the serum levels of total IgM or IgG at day 4 or 10 (Table 9). Thus the B cell homeostasis was not affected by the administration of any of the proteins.

TABLE 9
Total IgM and IgG serum levels
Absorption (OD450 ηm)

| | Total IgM | | Total IgG | |
|---|---|---|---|---|
| Experimental group | Day 4 | Day 10 | Day 4 | Day 10 |
| Control | 0.220 ± 0.014 | 0.236 ± 0.025 | 0.631 ± 0.057 | 0.667 ± 0.053 |
| mB7-H6-Fc | 0.292 ± 0.039 | 0.265 ± 0.018 | 0.628 ± 0.053 | 0.862 ± 0.072 |
| Naïve | 0.219 ± 0.023 | 0.307 ± 0.027 | 0.699 ± 0.026 | 0.730 ± 0.120 |

In summary the role mB7-H6 as negative regulator of T cell activation can explain the phenotype observed in vivo after administration of mB7-H6-Fc fusion protein. Already the strong inhibitory effect observed in vitro indicated the potential as negative regulator. Due to this property of mB7-H6 a significant downregulation of the immune response could be observed in vivo.

### EXAMPLE 18

Co-Stimulatory Effect of hsB7-H4LV on Lymphocyte Proliferation

To test whether hsB7-H4LV co-stimulates the proliferation of B cells and/or T cells, a co-stimulation assay is performed. In this assay purified B cells and/or T cells are stimulated by immobilized anti-human IgM and/or anti-CD3 antibody in the presence of immobilized B7-H4LV-Fc fusion protein. The proliferation of B cells and/or T cells is determined by [³H]-thymidine-incorporation after 72 hours of incubation. B7-H4LV-Fc fusion protein modulates lymphocyte proliferation in a dose-dependent fashion in the presence of a suboptimal dose of anti-human IgM and/or anti-CD3 antibody (coated onto the tissue culture plate).

### EXAMPLE 19

Stimulation of B Cell Proliferation by Human B7-H5

To test whether hsB7-H5 is a positive regulator of B cell proliferation, a B cell proliferation assay is performed (according to detailed description in example 13). In this assay purified human B cells are stimulated by immobilized anti-human IgM antibody in the presence of immobilized hsB7-H5-Fc or hsB7-H5-compFLAG fusion protein. The proliferation of B cells is determined by [³H]-thymidine-incorporation after 72 hours of incubation. The hsB7-H5 fusion protein increases B cell proliferation in a dose-dependent fashion in the presence of a suboptimal dose of anti-human IgM antibody (coated onto the tissue culture plate).

### EXAMPLE 20

Inhibitory Effect of hsB7-H6 on T Cell Proliferation

To test whether hsB7-H6 inhibites the proliferation T cells, a co-stimulation and inhibition assay is performed (according to detailed description in example 14). In these assays purified human T cells are stimulated by immobilized anti-CD3 antibody in the presence of immobilized hsB7-H6-Fc or hsB7-H6-compFLAG fusion protein (see example 11). The proliferation of T cells is determined by [³H]-thymidine-incorporation after 72 hours of incubation hsB7-H6 fusion proteins modulate lymphocyte proliferation in a dose-dependent fashion in the presence of a suboptimal dose of anti-CD3 antibody and/or anti-CD28 antibody (coated onto the tissue culture plate).

### EXAMPLE 21

Expression Cloning of Counter Receptor of the Novel B7-Family Members

To search for potential counter-receptors for hsB7-H4, mB7-H5, hsB7-H5, mB7-H6, and hsB7-H6, respectively, expression cloning screens are performed. For the screening the Fc or compFLAG fusion protein (described in example 3, 5, 7, 9, or 11) are used as bait. The expression cloning screenings for the corresponding counterreceptor are performed for example as described in the U.S. Pat. No. 6,524,792.

### EXAMPLE 22

In Vivo Modulation of the Acetylcholine Receptor Specific Lymphocyte Response

To demonstrate a role of mB7-H5 and mB7-H6 in antibody mediated autoimmune diseases in mice the experimental autoimmune myasthenia gravis (EAMG) is used. C57BL/6 mice are immunized with 20 μg of acetylcholine receptor (AChR) in CFA emulsion. Mice are injected i.p. with 500 μg of purified mB7-H5 protein, mB7-H6 protein, or control protein on days 0 and 3 after immunization. One group of mice is euthanized seven days after immunization, and lymph node cells (LNC) are collected. LNC are cultured with no antigen, AChR, or AChR α-chain peptide α₁₄₆₋₁₆₂. Proliferation is measured by [³H]thymidine incorporation. Second group of mice is boosted on day 30 with 20 μg of AChR in CFA and are injected i.p. with 500 μg of purified mB7-H5 protein, mB7-H6 protein, or control protein, respectively, on days 30 and 33 after immunization. These mice are assessed for the characteristic symptoms of EAMG, such as muscle weakness. Sera are collected on days 14 and 44 after the first immunization for the measurement of anti AChR antibody. At termination, LNC are collected, and their proliferative and cytokine responses to AchR and dominant peptide α₄₆₋₁₆₂ are assessed in vitro.

### EXAMPLE 23

Immunologic Effects of B7-H5 and B7-H6 Therapy in the Systemic Lupus Erythematosus Mouse Model

To determine the immunologic effect of mB7-H5, and mB7-H6 therapy the systemic lupus erythematosus mouse model is used. Five to six month old (NZB×NZW) F, mice are treated with continuous administration of mB7-H5, mB7-H6, and control protein. Mice are followed up clinically, and their spleens are studied at intervals for B and T cell numbers and subsets and frequency of anti-doublestranded DNA (anti-dsDNA)-producing B cells. T cell-dependent immunity is assessed by studying the humoral response to Qβp33xNKpt antigen. Female (NZB×NZW) F₁ mice are maintained in a conventional animal housing facility. In detail mice are treated at the age of 20 weeks or 26 weeks with 500 μg of purified mB7-H5 protein, mB7-H6 protein, control protein, or no protein given intraperitoneally weekly for 6 month until age 46 weeks. Prior to treatment, mice are randomized into treatment groups. Mice are bled every 2-4 weeks and anti-dsDNA antibody titers are determined by ELISA. Urine is tested for proteinuria by dipstick (Multistick; Fisher, Pittsburgh, Pa.) every 2 weeks. At different time groups of the experimental groups are sacrified and ELISpot assays for DNA-specific anti-IgM and anti-IgG forming cells is done. The spleen cells are analyzed by flow cytometry for B and T cell markers using different antibodies. Mice are followed up until death

All of the references cited herein, including patents, patent applications, and publications, are hereby incorporated in their entireties by reference.

While this invention has been described with an emphasis upon preferred embodiments, variations of the preferred embodiments can be used, and it is intended that the invention can be practiced otherwise than as specifically described herein. Accordingly, this invention includes all modifications encompassed within the spirit and scope of the invention as defined by the claims.

What is claimed is:

1. An isolated nucleic acid, wherein said nucleic acid is selected from the group consisting of:
(i) a nucleic acid comprising at least one of the nucleic acid sequences listed in SEQ ID NOs 1, 3, 5, 7, 9, 11, 13, 15, 41, and 43;
(ii) a nucleic acid having a sequence of at least 80% identity, preferably at least 90% identity, more preferred at least 95% identity, most preferred at least 98% identity with any of the nucleic acid sequences listed in SEQ ID NOs 1, 3, 5, 7, 9, 11, 13, 15, 41, and 43;
(iii) a nucleic acid that hybridizes to a nucleic acid of (i) or (ii);
(iv) a nucleic acid, wherein said nucleic acid is derivable by substitution, addition and/or deletion of one of the nucleic acids of (i), (ii) or (iii);
(v) a fragment of any of the nucleic acids of (i) to (iv), that hybridizes to a nucleic acid of (i).

2. The nucleic acid according to claim 1, wherein said nucleic acid is a DNA, a RNA or a PNA.

3. The nucleic acid according to claim 1, wherein said nucleic acid encodes a polypeptide that is capable of modulating an immune response, wherein preferably said immune response is a T cell response, a B cell response, or a T cell and a B cell response.

4. An isolated polypeptide comprising a polypeptide sequence encoded by a nucleic acid according to claim 1.

5. The polypeptide according to claim 4, wherein said polypeptide sequence is selected from the group consisting of:
(i) hsB7-H4LV (SEQ ID NO:2);
(ii) hsB7-H4LV(ECD) (SEQ ID NO:4);
(iii) hsB7-H5 (SEQ ID NO:6);
(iv) hsB7-H5(ECD) (SEQ ID NO:8);
(v) mB7-H5 (SEQ ID NO: 10);
(vi) mB7-H5(ECD) (SEQ ID NO: 12);
(vii) mB7-H6 (SEQ ID NO: 14);
(viii) mB7-H6(ECD) (SEQ ID NO: 16);
(ix) hsB7-H6 (SEQ ID NO:42);
(x) hsB7-H6(ECD) (SEQ ID NO:44) and;
(xi) a functional derivative of (i), (ii), (iii), (iv), (v), (vi), (vii), (viii), (ix), or (x).

6. The polypeptide according to claim 4, wherein said polypeptide is capable of modulating an immune response, wherein preferably said immune response is a T cell response, a B cell response, or a T cell and a B cell response.

7. The polypeptide according to claim 5, wherein said polypeptide is capable of modulating an immune response, wherein preferably said immune response is a T cell response, a B cell response, or a T cell and a B cell response.

8. A recombinant vector, comprising a nucleic acid according to claim 1.

9. A recombinant vector, wherein said recombinant vector is capable of producing a polypeptide according to claim 4.

10. A host cell comprising a nucleic acid according to claim 1.

11. An antibody that specifically binds a polypeptide according to claim 4.

12. An antibody directed against a polypeptide according to claim 4, wherein said antibody inhibits the polypeptides capability to modulate an immune response.

13. An antibody directed against a polypeptide according to claim 5, wherein said antibody inhibits the polypeptides capability to modulate an immune response.

14. A hybridoma cell line, expressing an antibody that specifically binds a polypeptide according to claim 4.

15. A transfected cell line capable of expressing the antibody according to claim 13.

16. A pharmaceutical composition comprising a polypeptide according to claim 4 and a pharmaceutically acceptable carrier.

17. A pharmaceutical composition comprising a polypeptide according to claim 5 and a pharmaceutically acceptable carrier.

18. A pharmaceutical composition comprising an antibody according to claim 13 and a pharmaceutically acceptable carrier.

19. A polypeptide according to claim 4 for use as a medicament.

20. A polypeptide according to claim 5 for use as a medicament.

21. An antibody according to claim 13 for use as a medicament.

22. Use of a polypeptide according to claim 4 for the preparation of a medicament for modulating the immune response.

23. Use of a polypeptide according to claim 5 for the preparation of a medicament for treating and/or preventing autoimmune diseases including, and preferably consisting of, type I diabetes and multiple sclerosis, asthma, arthritis, myasthenia gravis, arthritis, lupus erythematosus, pemhigus, psoriasis, colitis or rejection of transplanted organs, rejection of xenotransplants, immuno deficiency diseases, and cancer.

24. Use of an antibody according to claim 13 for the preparation of a medicament for treating and/or preventing autoimmune diseases including, and preferably consisting of, type I diabetes and multiple sclerosis, asthma, arthritis, myasthenia gravis, arthritis, lupus erythematosus, pemhigus, psoriasis, colitis or rejection of transplanted organs, rejection of xenotransplants, immuno deficiency diseases, and cancer.

25. A method of identifying a compound that modulates an immune response, which method comprises:
(i) contacting a B cell and/or T cell with a polypeptide according to claim 4 in the absence or presence of a compound of interest;
(ii) comparing the immune response in the absence of said compound of interest with the immune response in the presence of said compound of interest.

26. The method of claim 25, wherein the contacting step (i) is performed by contacting B cells, T cells, or B cells and T cells, with cells expressing said polypeptide, with a polypeptide that is matrix-bound, or with a free polypeptide.

27. A method of treating and/or preventing a disease in a mammal, wherein said disease is selected from autoimmune diseases and diseases that benefit from an enhanced or reduced immune response, preferably type I diabetes and multiple sclerosis, asthma, arthritis, psoriasis, colitis or rejection of transplanted organs, immuno deficiency diseases, or cancer, which method comprises administering to the mammal a therapeutically effective amount of the polypeptide according to claim 4.

28. A method of producing the polypeptide according to claim 4, said method comprising the steps of:
(i) providing the host cell according to claim;
(ii) culturing said host cell under conditions suitable for expression of said polypeptide; and
(iii) isolating said polypeptide from said host cell.

29. A method of producing an antibody, said method comprising the steps of:
(i) providing the hybridoma cell according to claim 14;
(ii) culturing said hybridoma cell under conditions suitable for expression of said antibody; and
(iii) isolating said antibody from said hybridoma cell.

30. A method of producing an antibody, said method comprising the steps of:
(i) providing a cell line transfected to express said antibody according to claim 15;
(ii) culturing said cell line under conditions suitable for expression of said antibody; and
(iii)isolating said antibody from said cell line.


SEQUENCE LISTING (50 sequences)

SEQ ID NO: 1 - DNA, 970 bp, homo sapiens
  CDS (15)..(959)
actgctgacgagagatggtggacctctcagtctccccagactccttgaagccagtatcgctgaccagcagtcttgtcttcctcatgcacctcctcctccttcagcctggggagccgagctcagaggtcaaggtgctaggccctgagtatcccatcctggccctcgtcggggaggaggtggagttcccgtgccacctatggccacagctggatgcccagcaaatggagatccgctggttccggagtcagaccttcaatgtggtacacctgtaccaggagcagcaggagctccctggcaggcagatgccggcgttccggaacaggaccaagttggtcaaggacgacatcgcctatggcagcgtggtcctgcagcttcacagcatcatcccctctgacaagggcacatatggctgccgcttccactccgacaacttctctggcgaagctctctgggaactggaggtagcagggctgggctcagaccctcacctctcccttgagggcttcaaggaaggaggcattcagctgaggctcagatccagtggctggtaccccaagcctaaggttcagtggagagaccaccagggacagtgcctgcctccagagtttgaagccatcgtctgggatgcccaggacctgttcagtctggaaacatctgtggttgtccgagcgggagccctcagcaatgtgtccgtctccatccagaatctcctcttgagccagaagaaagagttggtggtccagatagcagacgtgttcgtacccggagcctctgcgtggaagagcgcgttcgtcgcgaccctgccgctgctgttggtcctcgcggcgctggcgctgggcgtcctccggaagcagcggagaagccgagaaaagctgaggaagcaggcggagaagagacaaggtgagcggggacagggcgttctgcacgcacctgcccaagtgccaaaacccgccgtcatctaaaggctgtg

SEQ ID NO: 2 - protein, 315 aa, homo sapiens
MVDLSVSPDSLKPVSLTSSLVFLMHLLLLQPGEPSSEVKVLGPEYPILALVGEEVEFPCHLWPQLDAQQMEIRWFRSQTFNVVHLYQEQQELPGRQMPAFRNRTKLVKDDIAYGSVVLQLHSIIPSDKGTYGCRFHSDNFSGEALWELEVAGLGSDPHLSLEGFKEGGIQLRLRSSGWYPKPKVQWRDHQGQCLPPEFEAIVWDAQDLFSLETSVVVRAGALSNVSVSIQNLLLSQKKELVVQIADVFVPGASAWKSAFVATLPLLLVLAALALGVLRKQRRSREKLRKQAEKRQGERGQGVLHAPAQVPKPAVI

SEQ ID NO: 3 - DNA, 781 bp, homo sapiens
  CDS (22)..(780)
gggggtacctgctgacgagagatggtggacctctcagtctccccagactccttgaagccagtatcgctgaccagcagtcttgtcttcctcatgcacctcctcctccttcagcctggggagccgagctcagaggtcaaggtgctaggccctgagtatcccatcctggccctcgtcggggaggaggtggagttcccgtgccacctatggccacagctggatgcccagcaaatggagatccgctggttccggagtcagaccttcaatgtggtacacctgtaccaggagcagcaggagctccctggcaggcagatgccggcgttccggaacaggaccaagttggtcaaggacgacatcgcctatggcagcgtggtcctgcagcttcacagcatcatcccctctgacaagggcacatatggctgccgcttccactccgacaacttctctggcgaagctctctgggaactggaggtagcagggctgggctcagaccctcacctctcccttgagggcttcaaggaaggaggcattcagctgaggctcagatccagtggctggtaccccaagcctaaggttcagtggagagaccaccagggacagtgcctgcctccagagtttgaagccatcgtctgggatgcccaggacctgttcagtctggaaacatctgtggttgtccgagcgggagccctcagcaatgtgtccgtctccatccagaatctcctcttgagccagaagaaagagttggtggtccagatagcagacgtgttcgtacccgggctagccg

SEQ ID NO: 4 - protein, 253 aa, homo sapiens
MVDLSVSPDSLKPVSLTSSLVFLMHLLLLQPGEPSSEVKVLGPEYPILALVGEEVEFPCHLWPQLDAQQMEIRWFRSQTFNVVHLYQEQQELPGRQMPAFRNRTKLVKDDIAYGSVVLQLHSIIPSDKGTYGCRFHSDNFSGEALWELEVAGLGSDPHLSLEGFKEGGIQLRLRSSGWYPKPKVQWRDHQGQCLPPEFEAIVWDAQDLFSLETSVVVRAGALSNVSVSIQNLLLSQKKELVVQIADVFVPGLA

SEQ ID NO: 5 - DNA, 1905 bp, homo sapiens
  CDS (175)..(1464)
ccgcagtgtgtgagaaagaggccctctctcagatgaatggataaagaaaatgcaggacatatggggggaggagccaagatggccgaataggaacagctccggtctacagctcccagtgtgagcgacacagaagacaggtgatttctgcatttccatctgaggcaagaagaataaatgtctctggtggaacttttgctctggtggaactgcttttctagaactggtgttgcagcatccctggaagtgtcagagagccctgggagtatccaggtggcccggggtcagacagcagtcctgccctgcactttcactaccagcgctgccctcattaacctcaatgtcatttggatggtcactcctctctccaatgccaaccaacctgaacaggtcatcctgtatcagggtggacagatgtttgatggtgccccccggttccacggtagggtaggatttacaggcaccatgccagctaccaatgtctctatcttcattaataacactcagttatcagacactggcacctaccagtgcctggtcaacaaccttccagacatagggggcaggaacattggggtcaccggtctcacagtgttagttcccccttctgccccacactgccaaatccaaggatcccaggatattggcagcgatgtcatcctgctctgtagctcagaggaaggcattcctcgaccaacttacctttgggagaagttagacaataccctcaaactacctccaacagctactcaggaccaggtccagggaacagtcaccatccggaacatcagtgccctgtcttcaggtttgtaccagtgcgtggcttctaatgctattggaaccagcacctgtcttctggatctccaggttatttcaccccagcccaggaacattggactaatagctggagccattggcactggtgcagttattatcattttttgcattgcactaattttaggggcattcttttactggagaagcaaaaataaagaggaggaagaagaagaaattcctaatgaaataagagaggatgatcttccacccaagtgttcttctgccaaagcatttcacactgagatttcctcctcggacaacaacacactaacctcttccaatgcctacaacagtcgatactggagcaacaatccaaaagttcatagaaacacagagtcagtcagccacttcagtgacttgggccaatctttctctttccactcaggcaatgccaacataccatccatttatgctaatgggacccatctggtcccgggtcaacataagactctggtagtgacagccaacagagggtcatcaccacaggtgatgtccaggagcaatggctcagtcagtaggaagcctcggcctccacacactcattcctacaccatcagccacgcaacactggaacgaattggtgcagtacctgtcatggtaccagcccagagtcgggccgggtccttggtataggacatgaggaaatgttgtgttcagaaatgaataaatggaatgccctcatacaagggggagggtggggtggggagtgctgggaaagaaacacttccttataattatattagtaaaatgcacaaagaagaaggcagtgctgttacttggccactaagatgtgtaaaatggactgaaatgctccatcatgaagacttgcttccccaccaaagatgtcctgggattctgctggatctcaaagatgtgccaagccaaggaaaaagatacaagagcagaatagtacttaaaatccaaactgccgcccagatgggcttgttcttcatgcctaacttaataatttttaagagattaaagtgccagatggagtttaaaaaaaaaaaaaaaaaaaaaaaaaaaaaaaaaaaaaaaaaaaaaaaaaaaaaaaaaaaaaaaaaaaaaaaa

SEQ ID NO: 6 - protein, 430 aa, homo sapiens
MSLVELLLWWNCFSRTGVAASLEVSESPGSIQVARGQTAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLIAGAIGTGAVIIIFCIALILGAFFYWRSKNKEEEEEEIPNEIREDDLPPKCSSAKAFHTEISSSDNNTLTSSNAYNSRYWSNNPKVHRNTESVSHFSDLGQSFSFHSGNANIPSIYANGTHLVPGQHKTLVVTANRGSSPQVMSRSNGSVSRKPRPPHTHSYTISHATLERIGAVPVMVPAQSRAGSLV

SEQ ID NO: 7 - DNA, 735 bp, homo sapiens
  CDS (9)..(734)
ggggtaccatgtctctggtggaacttttgctctggtggaactgcttttctagaactggtgttgcagcatccctggaagtgtcagagagccctgggagtatccaggtggcccggggtcagacagcagtcctgccctgcactttcactaccagcgctgccctcattaacctcaatgtcatttggatggtcactcctctctccaatgccaaccaacctgaacaggtcatcctgtatcagggtggacagatgtttgatggtgccccccggttccacggtagggtaggatttacaggcaccatgccagctaccaatgtctctatcttcattaataacactcagttatcagacactggcacctaccagtgcctggtcaacaaccttccagacatagggggcaggaacattggggtcaccggtctcacagtgttagttcccccttctgccccacactgccaaatccaaggatcccaggatattggcagcgatgtcatcctgctctgtagctcagaggaaggcattcctcgaccaacttacctttgggagaagttagacaataccctcaaactacctccaacagctactcaggaccaggtccagggaacagtcaccatccggaacatcagtgccctgtcttcaggtttgtaccagtgcgtggcttctaatgctattggaaccagcacctgtcttctggatctccaggttatttcaccccagcccaggaacattgggctagccg

SEQ ID NO: 8 - protein, 242 aa, homo sapiens
MSLVELLLWWNCFSRTGVAASLEVSESPGSIQVARGQTAVLPCTFTTSAALINLNVIWMVTPLSNANQPEQVILYQGGQMFDGAPRFHGRVGFTGTMPATNVSIFINNTQLSDTGTYQCLVNNLPDIGGRNIGVTGLTVLVPPSAPHCQIQGSQDIGSDVILLCSSEEGIPRPTYLWEKLDNTLKLPPTATQDQVQGTVTIRNISALSSGLYQCVASNAIGTSTCLLDLQVISPQPRNIGLA

SEQ ID NO: 9 - DNA, 1395 bp, Mus musculus
  CDS (64)..(1347)
cctacgctgctaccccgtccgcccaggagcccggcggacggcggctcccccggcggctccggcatgactcggcggcgctccgctccggcgtcctggctgctcgtgtcgctgctcggtgtcgcaacatccctggaagtgtccgagagcccaggcagtgtccaggtggcccggggccagacagcagtcctgccctgcgccttctccaccagtgctgccctcctgaacctcaatgtcatttggatggtcattcccctctccaatgcaaaccagcccgaacaggtcattctttatcagggtggacaaatgtttgacggcgccctccggttccacgggagggtaggatttaccggcaccatgcctgctaccaatgtctcgatcttcatcaataacacacagctgtcagatacgggcacgtaccagtgcttggtgaataaccttccagacagagggggcagaaacatcggggtcactggcctcacagtgttagtccccccttctgctccacaatgccaaatccaaggatcccaggacctcggcagtgacgtcatccttctgtgtagttcagaggaaggcatccctcggcccacgtacctttgggagaagttagataatacgctcaagctacctccaacagccactcaggaccaggtccagggaacagtcaccatccggaatatcagtgccctctcttccggtctgtaccagtgtgtggcttctaatgccatcgggaccagcacctgtctgctggacctccaggttatctcaccccagccccggagcgttggagtaatagccggagcggttggcaccggtgctgttcttatcgtcatctgccttgcactaatttcaggggcgttcttttactggagaagcaaaaacaaagaggaggaggaggaagaaattcctaatgaaatcagagaggatgatcttccccctaaatgctcttctgccaaagccttccacacggagatatcctcctcagaaaataacacgctgacctcttccaatacctacaacagtcgatactggaacaacaatccaaaaccccatagaaacacagagtctttcaaccacttcagtgacttacgccagtctttctctggcaatgcagttatcccatcaatctatgcaaatgggaaccatctggttttgggtccacataagactctggtagttacagccaacagagggtcatcacctcaggtcttgcccaggaacaatggttcagtcagcaggaagccttggcctcaacacactcattcctacacagtaagccaaatgaccctggagcgcatcggtgcagtgcctgtcatggtgcctgcccagagtcgagcagggtccctggtataggatgactgaggaaaccatgttcagaagagaataaatggaccgcct

SEQ ID NO: 10 - protein, 428 aa, Mus musculus
MTRRRSAPASWLLVSLLGVATSLEVSESPGSVQVARGQTAVLPCAFSTSAALLNLNVIWMVIPLSNANQPEQVILYQGGQMFDGALRFHGRVGFTGTMPATNVSIFINNTQLSDTGTYQCLVNNLPDRGGRNIGVTGLTVLVPPSAPQCQIQGSQDLGSDVILLCSSEEGIPRPTYLWEKLDNTLKLPPTATQDQVQGTVTIRNISALSSGLYQCVASNAIGTSTCLLDLQVISPQPRSVGVIAGAVGTGAVLIVICLALISGAFFYWRSKNKEEEEEEIPNEIREDDLPPKCSSAKAFHTEISSSENNTLTSSNTYNSRYWNNNPKPHRNTESFNHFSDLRQSFSGNAVIPSIYANGNHLVLGPHKTLVVTANRGSSPQVLPRNNGSVSRKPWPQHTHSYTVSQMTLERIGAVPVMVPAQSRAGSLV

SEQ ID NO: 11 - DNA, 723 bp, Mus musculus
  CDS (9)..(722)
ggggtaccatgactcggcggcgctccgctccggcgtcctggctgctcgtgtcgctgctcggtgtcgcaacatccctggaagtgtccgagagcccaggcagtgtccaggtggcccggggccagacagcagtcctgccctgcgccttctccaccagtgctgccctcctgaacctcaatgtcatttggatggtcattcccctctccaatgcaaaccagcccgaacaggtcattctttatcagggtggacaaatgtttgacggcgccctccggttccacgggagggtaggatttaccggcaccatgcctgctaccaatgtctcgatcttcatcaataacacacagctgtcagatacgggcacgtaccagtgcttggtgaataaccttccagacagagggggcagaaacatcggggtcactggcctcacagtgttagtccccccttctgctccacaatgccaaatccaaggatcccaggacctcggcagtgacgtcatccttctgtgtagttcagaggaaggcatccctcggcccacgtacctttgggagaagttagataatacgctcaagctacctccaacagccactcaggaccaggtccagggaacagtcaccatccggaatatcagtgccctctcttccggtctgtaccagtgtgtggcttctaatgccatcgggaccagcacctgtctgctggacctccaggttatctcacccgtgctagccc

SEQ ID NO: 12 - protein, 238 aa, Mus musculus
MTRRRSAPASWLLVSLLGVATSLEVSESPGSVQVARGQTAVLPCAFSTSAALLNLNVIWMVIPLSNANQPEQVILYQGGQMFDGALRFHGRVGFTGTMPATNVSIFINNTQLSDTGTYQCLVNNLPDRGGRNIGVTGLTVLVPPSAPQCQIQGSQDLGSDVILLCSSEEGIPRPTYLWEKLDNTLKLPPTATQDQVQGTVTIRNISALSSGLYQCVASNAIGTSTCLLDLQVISPVLA

SEQ ID NO: 13 - DNA, 867 bp, Mus musculus
  CDS (16)..(855)
gtagcttcaaataggatggagatctcatcaggcttgctgttcctgggccacctaatagtgctcacctatggccaccccaccctaaaaacacctgagagtgtgacagggacctggaaaggagatgtgaagattcagtgcatctatgatcccctgagaggctacaggcaagttttggtgaaatggctggtaagacacggctctgactccgtcaccatcttcctacgtgactccactggagaccatatccagcaggcaaagtacagaggccgcctgaaagtgagccacaaagttccaggagatgtgtccctccaaataaataccctgcagatggatgacaggaatcactatacatgtgaggtcacctggcagactcctgatggaaaccaagtaataagagataagatcattgagctccgtgttcggaaatataatccacctagaatcaatactgaagcacctacaaccctgcactcctctttggaagcaacaactataatgagttcaacctctgacttgaccactaatgggactggaaaacttgaggagaccattgctggttcagggaggaacctgccaatctttgccataatcttcatcatctccctttgctgcatagtagctgtcaccataccttatatcttgttccgctgcaggacattccaacaagagtatgtctatggagtgagcagggtgtttgccaggaagacaagcaactctgaagaaaccacaagggtgactaccatcgcaactgatgaaccagattcccaggctctgattagtgactactctgatgatccttgcctcagccaggagtaccaaataaccatcagatcaacaatgtctattcctgcctgctgaacacagttt

SEQ ID NO: 14 - protein, 280 aa, Mus musculus
MEISSGLLFLGHLIVLTYGHPTLKTPESVTGTWKGDVKIQCIYDPLRGYRQVLVKWLVRHGSDSVTIFLRDSTGDHIQQAKYRGRLKVSHKVPGDVSLQINTLQMDDRNHYTCEVTWQTPDGNQVIRDKIIELRVRKYNPPRINTEAPTTLHSSLEATTIMSSTSDLTTNGTGKLEETIAGSGRNLPIFAIIFIISLCCIVAVTIPYILFRCRTFQQEYVYGVSRVFARKTSNSEETTRVTTIATDEPDSQALISDYSDDPCLSQEYQITIRSTMSIPAC

SEQ ID NO: 15 - DNA, 574 bp, Mus musculus
  CDS (11)..(574)
gggtaccaggatggagatctcatcaggcttgctgttcctgggccacctaatagtgctcacctatggccaccccaccctaaaaacacctgagagtgtgacagggacctggaaaggagatgtgaagattcagtgcatctatgatcccctgagaggctacaggcaagttttggtgaaatggctggtaagacacggctctgactccgtcaccatcttcctacgtgactccactggagaccatatccagcaggcaaagtacagaggccgcctgaaagtgagccacaaagttccaggagatgtgtccctccaaataaataccctgcagatggatgacaggaatcactatacatgtgaggtcacctggcagactcctgatggaaaccaagtaataagagataagatcattgagctccgtgttcggaaatataatccacctagaatcaatactgaagcacctacaaccctgcactcctctttggaagcaacaactataatgagttcaacctctgacttgaccactaatgggactggaaaacttgaggagaccattgctggttcagggaggaacctgctagcc

SEQ ID NO: 16 - protein, 188 aa, Mus musculus
MEISSGLLFLGHLIVLTYGHPTLKTPESVTGTWKGDVKIQCIYDPLRGYRQVLVKWLVRHGSDSVTIFLRDSTGDHIQQAKYRGRLKVSHKVPGDVSLQINTLQMDDRNHYTCEVTWQTPDGNQVIRDKIIELRVRKYNPPRINTEAPTTLHSSLEATTIMSSTSDLTTNGTGKLEETIAGSGRNLLA

SEQ ID NO: 17 - DNA, 11006 bp, Artificial Sequence
  pCEP-hsB7-H4(ECD)-Fc
gccccgccgccggacgaactaaacctgactacggcatctctgccccttcttcgctggtacgaggagcgcttttgttttgtattcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcatgctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcgccgcgtgcggctgctggagatggcggacgcgatggatatgttctgccaagggttggtttgcgcattcacagttctccgcaagaattgattggctccaattcttggagtggtgaatccgttagcgaggccatccagcctcgcgtcgaactagatgatccgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggagggtgaccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgaccccgaccgccacatcgaacgcgtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgacccggtccgacggcggcccacgggtcccaggggggtcgacctcgaaacttgtttattgcagcttataatggttacaaataaagcaatagcatcacaaatttcacaaataaagcatttttttcactgcattctagttgtggtttgtccaaactcatcaatgtatcttatcatgtctggatcgatccgaaccccttcctcgaccaattctcatgtttgacagcttatcatcgcagatccgggcaacgttgttgcattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtacctgctgacgagagatggtggacctctcagtctccccagactccttgaagccagtatcgctgaccagcagtcttgtcttcctcatgcacctcctcctccttcagcctggggagccgagctcagaggtcaaggtgctaggccctgagtatcccatcctggccctcgtcggggaggaggtggagttcccgtgccacctatggccacagctggatgcccagcaaatggagatccgctggttccggagtcagaccttcaatgtggtacacctgtaccaggagcagcaggagctccctggcaggcagatgccggcgttccggaacaggaccaagttggtcaaggacgacatcgcctatggcagcgtggtcctgcagcttcacagcatcatcccctctgacaagggcacatatggctgccgcttccactccgacaacttctctggcgaagctctctgggaactggaggtagcagggctgggctcagaccctcacctctcccttgagggcttcaaggaaggaggcattcagctgaggctcagatccagtggctggtaccccaagcctaaggttcagtggagagaccaccagggacagtgcctgcctccagagtttgaagccatcgtctgggatgcccaggacctgttcagtctggaaacatctgtggttgtccgagcgggagccctcagcaatgtgtccgtctccatccagaatctcctcttgagccagaagaaagagttggtggtccagatagcagacgtgttcgtacccgggctagcgatcgaaggtcgcaagcttactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgaggcccgaacaaaaactcatctcagaagaggatctgaatagcgccgtcgaccatcatcatcatcatcattgagtttnaacgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtggggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaccggtcgactctaga

SEQ ID NO: 18 - DNA, 10561 bp, Artificial Sequence
  pCEP-hsB7-H4(ECD)-comp-FL-C
gccccgccgccggacgaactaaacctgactacggcatctctgccccttcttcgctggtacgaggagcgcttttgttttgtattcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcgccgcgtgcggctgctggagatggcggacgcgatggatatgttctgccaagggttggtttgcgcattcacagttctccgcaagaattgattggctccaattcttggagtggtgaatccgttagcgaggccatccagcctcgcgtcgaactagatgatccgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggagggtgaccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgaccccgaccgccacatcgaacgcgtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgacccggtccgacggcggcccacgggtcccaggggggtcgacctcgaaacttgtttattgcagcttataatggttacaaataaagcaatagcatcacaaatttcacaaataaagcatttttttcactgcattctagttgtggtttgtccaaactcatcaatgtatcttatcatgtctggatcgatccgaaccccttcctcgaccaattctcatgtttgacagcttatcatcgcagatccgggcaacgttgttgcattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtacctgctgacgagagatggtggacctctcagtctccccagactccttgaagccagtatcgctgaccagcagtcttgtcttcctcatgcacctcctcctccttcagcctggggagccgagctcagaggtcaaggtgctaggccctgagtatcccatcctggccctcgtcggggaggaggtggagttcccgtgccacctatggccacagctggatgcccagcaaatggagatccgctggttccggagtcagaccttcaatgtggtacacctgtaccaggagcagcaggagctccctggcaggcagatgccggcgttccggaacaggaccaagttggtcaaggacgacatcgcctatggcagcgtggtcctgcagcttcacagcatcatcccctctgacaagggcacatatggctgccgcttccactccgacaacttctctggcgaagctctctgggaactggaggtagcagggctgggctcagaccctcacctctcccttgagggcttcaaggaaggaggcattcagctgaggctcagatccagtggctggtaccccaagcctaaggttcagtggagagaccaccagggacagtgcctgcctccagagtttgaagccatcgtctgggatgcccaggacctgttcagtctggaaacatctgtggttgtccgagcgggagccctcagcaatgtgtccgtctccatccagaatctcctcttgagccagaagaaagagttggtggtccagatagcagacgtgttcgtacccgggctagcgcagccgcagccgaaaccgcagccgcagccgcagccgcagccgaaaccgcagccgaaaccggaaccggaagctttgggagactgctgcccacagatgcttcgagaactccaggagactaatgcggcgctgcaagacgtgagagagctcttgcgacagcaggtcaaggagatcaccttcctgaagaatacggtgatggaatgtgacgcttgcggaggatctggtctagacgactacaaggatgacgacgacaagtaggggcccgaacaaaaactcatctcagaagaggatctgaatagcgccgtcgaccatcatcatcatcatcattgagtttaaacgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtggggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaccggtcgactctaga

SEQ ID NO: 19 - DNA, 10961 bp, Artificial Sequence
  pCEP-hsB7-H5(ECD)-Fc
gccccgccgccggacgaactaaacctgactacggcatctctgccccttcttcgctggtacgaggagcgcttttgttttgtattcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcatgctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcgccgcgtgcggctgctggagatggcggacgcgatggatatgttctgccaagggttggtttgcgcattcacagttctccgcaagaattgattggctccaattcttggagtggtgaatccgttagcgaggccatccagcctcgcgtcgaactagatgatccgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggagggtgaccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgaccccgaccgccacatcgaacgcgtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgacccggtccgacggcggcccacgggtcccaggggggtcgacctcgaaacttgtttattgcagcttataatggttacaaataaagcaatagcatcacaaatttcacaaataaagcatttttttcactgcattctagttgtggtttgtccaaactcatcaatgtatcttatcatgtctggatcgatccgaaccccttcctcgaccaattctcatgtttgacagcttatcatcgcagatccgggcaacgttgttgcattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccatgtctctggtggaacttttgctctggtggaactgcttttctagaactggtgttgcagcatccctggaagtgtcagagagccctgggagtatccaggtggcccggggtcagacagcagtcctgccctgcactttcactaccagcgctgccctcattaacctcaatgtcatttggatggtcactcctctctccaatgccaaccaacctgaacaggtcatcctgtatcagggtggacagatgtttgatggtgccccccggttccacggtagggtaggatttacaggcaccatgccagctaccaatgtctctatcttcattaataacactcagttatcagacactggcacctaccagtgcctggtcaacaaccttccagacatagggggcaggaacattggggtcaccggtctcacagtgttagttcccccttctgccccacactgccaaatccaaggatcccaggatattggcagcgatgtcatcctgctctgtagctcagaggaaggcattcctcgaccaacttacctttgggagaagttagacaataccctcaaactacctccaacagctactcaggaccaggtccagggaacagtcaccatccggaacatcagtgccctgtcttcaggtttgtaccagtgcgtggcttctaatgctattggaaccagcacctgtcttctggatctccaggttatttcaccccagcccaggaacattgggctagcgatcgaaggtcgcaagcttactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgaggcccgaacaaaaactcatctcagaagaggatctgaatagcgccgtcgaccatcatcatcatcatcattgagtttnaacgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtggggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaccggtcgactctaga

SEQ ID NO: 20 - DNA, 10516 bp, Artificial Sequence
  pCEP-hsB7-H5(ECD)-comp-FL-C
gccccgccgccggacgaactaaacctgactacggcatctctgccccttcttcgctggtacgaggagcgcttttgttttgtattcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcgccgcgtgcggctgctggagatggcggacgcgatggatatgttctgccaagggttggtttgcgcattcacagttctccgcaagaattgattggctccaattcttggagtggtgaatccgttagcgaggccatccagcctcgcgtcgaactagatgatccgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggagggtgaccgccacgaccggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgaccccgaccgccacatcgaacgcgtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgacccggtccgacggcggcccacgggtcccaggggggtcgacctcgaaacttgtttattgcagcttataatggttacaaataaagcaatagcatcacaaatttcacaaataaagcatttttttcactgcattctagttgtggtttgtccaaactcatcaatgtatcttatcatgtctggatcgatccgaaccccttcctcgaccaattctcatgtttgacagcttatcatcgcagatccgggcaacgttgttgcattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccatgtctctggtggaacttttgctctggtggaactgcttttctagaactggtgttgcagcatccctggaagtgtcagagagccctgggagtatccaggtggcccggggtcagacagcagtcctgccctgcactttcactaccagcgctgccctcattaacctcaatgtcatttggatggtcactcctctctccaatgccaaccaacctgaacaggtcatcctgtatcagggtggacagatgtttgatggtgccccccggttccacggtagggtaggatttacaggcaccatgccagctaccaatgtctctatcttcattaataacactcagttatcagacactggcacctaccagtgcctggtcaacaaccttccagacatagggggcaggaacattggggtcaccggtctcacagtgttagttcccccttctgccccacactgccaaatccaaggatcccaggatattggcagcgatgtcatcctgctctgtagctcagaggaaggcattcctcgaccaacttacctttgggagaagttagacaataccctcaaactacctccaacagctactcaggaccaggtccagggaacagtcaccatccggaacatcagtgccctgtcttcaggtttgtaccagtgcgtggcttctaatgctattggaaccagcacctgtcttctggatctccaggttatttcaccccagcccaggaacattgggctagcgcagccgcagccgaaaccgcagccgcagccgcagccgcagccgaaaccgcagccgaaaccggaaccggaagctttgggagactgctgcccacagatgcttcgagaactccaggagactaatgcggcgctgcaagacgtgagagagctcttgcgacagcaggtcaaggagatcaccttcctgaagaatacggtgatggaatgtgacgcttgcggaggatctggtctagacgactacaaggatgacgacgacaagtaggggcccgaacaaaaactcatctcagaagaggatctgaatagcgccgtcgaccatcatcatcatcatcattgagtttaaacgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtggggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaccggtcgactctaga

SEQ ID NO: 21 - DNA, 10921 bp, Artificial Sequence
  pCEP-mB7-H5(ECD)-Fc
ggatcgatccccgccgccggacgaactaaacctgactacggcatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcgccgcgtgcggctgctggagatggcggacgcgatggatatgttctgccaagggttggtttgcgcattcacagttctccgcaagaattgattggctccaattcttggagtggtgaatccgttagcgaggccatccagcctcgcgtcgaactagatgatccgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggagggtgaccgccacgaggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgaccccgaccgccacatcgaacgcgtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgacccggtccgacggcggcccacgggtcccaggggggtcgacctcgaaacttgtttattgcagcttataatggttacaaataaagcaatagcatcacaaatttcacaaataaagcatttttttcactgcattctagttgtggtttgtccaaactcatcaatgtatcttatcatgtctggatcgatccgaaccccttcctcgaccaattctcatgtttgacagcttatcatcgcagatccgggcaacgttgttgcattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccatgactcggcggcgctccgctccggcgtcctggctgctcgtgtcgctgctcggtgtcgcaacatccctggaagtgtccgagagcccaggcagtgtccaggtggcccggggccagacagcagtcctgccctgcgccttctccaccagtgctgccctcctgaacctcaatgtcatttggatggtcattcccctctccaatgcaaaccagcccgaacaggtcattctttatcagggtggacaaatgtttgacggcgccctccggttccacgggagggtaggatttaccggcaccatgcctgctaccaatgtctcgatcttcatcaataacacacagctgtcagatacgggcacgtaccagtgcttggtgaataaccttccagacagagggggcagaaacatcggggtcactggcctcacagtgttagtccccccttctgctccacaatgccaaatccaaggatcccaggacctcggcagtgacgtcatccttctgtgtagttcagaggaaggcatccctcggcccacgtacctttgggagaagttagataatacgctcaagctacctccaacagccactcaggaccaggtccagggaacagtcaccatccggaatatcagtgccctctcttccggtctgtaccagtgtgtggcttctaatgccatcgggaccagcacctgtctgctggacctccaggttatctcacccgtgctagcgatcgaaggtcgcaagcttactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgaggcccgaacaaaaactcatctcagaagaggatctgaatagcgccgtcgaccatcatcatcatcatcattgagtttaacgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtggggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaggtcgactctaga

SEQ ID NO: 22 - DNA, 10477 bp, Artificial Sequence
  pCEP-mB7-H5(ECD)-comp-FL-C
ggatcgatccccgccgccggacgaactaaacctgactacggcatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcgccgcgtgcggctgctggagatggcggacgcgatggatatgttctgccaagggttggtttgcgcattcacagttctccgcaagaattgattggctccaattcttggagtggtgaatccgttagcgaggccatccagcctcgcgtcgaactagatgatccgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggagggtgaccgccacgaggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgaccccgaccgccacatcgaacgcgtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgacccggtccgacggcggcccacgggtcccaggggggtcgacctcgaaacttgtttattgcagcttataatggttacaaataaagcaatagcatcacaaatttcacaaataaagcatttttttcactgcattctagttgtggtttgtccaaactcatcaatgtatcttatcatgtctggatcgatccgaaccccttcctcgaccaattctcatgtttgacagcttatcatcgcagatccgggcaacgttgttgcattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccatgactcggcggcgctccgctccggcgtcctggctgctcgtgtcgctgctcggtgtcgcaacatccctggaagtgtccgagagcccaggcagtgtccaggtggcccggggccagacagcagtcctgccctgcgccttctccaccagtgctgccctcctgaacctcaatgtcatttggatggtcattcccctctccaatgcaaaccagcccgaacaggtcattctttatcagggtggacaaatgtttgacggcgccctccggttccacgggagggtaggatttaccggcaccatgcctgctaccaatgtctcgatcttcatcaataacacacagctgtcagatacgggcacgtaccagtgcttggtgaataaccttccagacagagggggcagaaacatcggggtcactggcctcacagtgttagtccccccttctgctccacaatgccaaatccaaggatcccaggacctcggcagtgacgtcatccttctgtgtagttcagaggaaggcatccctcggcccacgtacctttgggagaagttagataatacgctcaagctacctccaacagccactcaggaccaggtccagggaacagtcaccatccggaatatcagtgccctctcttccggtctgtaccagtgtgtggcttctaatgccatcgggaccagcacctgtctgctggacctccaggttatctcacccgtgctagcgcagccgcagccgaaaccgcagccgcagccgcagccgcagccgaaaccgcagccgaaaccggaaccggaagctttgggagactgctgcccacagatgcttcgagaactccaggagactaatgcggcgctgcaagacgtgagagagctcttgcgacagcaggtcaaggagatcaccttcctgaagaatacggtgatggaatgtgacgcttgcggaggatctggtctagacgactacaaggatgacgacgacaagtaggggcccgaacaaaaactcatctcagaagaggatctgaatagcgccgtcgaccatcatcatcatcatcattgagtttaaacgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtggggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaggtcgactctaga

SEQ ID NO: 23 - DNA, 10774 bp, Artificial Sequence
  pCEP-mB7-H6(ECD)-Fc
ggatcgatccccgccgccggacgaactaaacctgactacggcatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcgccgcgtgcggctgctggagatggcggacgcgatggatatgttctgccaagggttggtttgcgcattcacagttctccgcaagaattgattggctccaattcttggagtggtgaatccgttagcgaggccatccagcctcgcgtcgaactagatgatccgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggagggtgaccgccacgaggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgaccccgaccgccacatcgaacgcgtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgacccggtccgacggcggcccacgggtcccaggggggtcgacctcgaaacttgtttattgcagcttataatggttacaaataaagcaatagcatcacaaatttcacaaataaagcatttttttcactgcattctagttgtggtttgtccaaactcatcaatgtatcttatcatgtctggatcgatccgaaccccttcctcgaccaattctcatgtttgacagcttatcatcgcagatccgggcaacgttgttgcattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccaggatggagatctcatcaggcttgctgttcctgggccacctaatagtgctcacctatggccaccccaccctaaaaacacctgagagtgtgacagggacctggaaaggagatgtgaagattcagtgcatctatgatcccctgagaggctacaggcaagttttggtgaaatggctggtaagacacggctctgactccgtcaccatcttcctacgtgactccactggagaccatatccagcaggcaaagtacagaggccgcctgaaagtgagccacaaagttccaggagatgtgtccctccaaataaataccctgcagatggatgacaggaatcactatacatgtgaggtcacctggcagactcctgatggaaaccaagtaataagagataagatcattgagctccgtgttcggaaatataatccacctagaatcaatactgaagcacctacaaccctgcactcctctttggaagcaacaactataatgagttcaacctctgacttgaccactaatgggactggaaaacttgaggagaccattgctggttcagggaggaacctgctagcgatcgaaggtcgcaagcttactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgaggcccgaacaaaaactcatctcagaagaggatctgaatagcgccgtcgaccatcatcatcatcatcattgagtttaacgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtggggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaggtcgactctaga

SEQ ID NO: 24 - DNA, 10330 bp, Artificial Sequence
  pCEP-mB7-H6(ECD)-comp-FL-C
ggatcgatccccgccgccggacgaactaaacctgactacggcatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcgccgcgtgcggctgctggagatggcggacgcgatggatatgttctgccaagggttggtttgcgcattcacagttctccgcaagaattgattggctccaattcttggagtggtgaatccgttagcgaggccatccagcctcgcgtcgaactagatgatccgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggagggtgaccgccacgaggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgaccccgaccgccacatcgaacgcgtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgacccggtccgacggcggcccacgggtcccaggggggtcgacctcgaaacttgtttattgcagcttataatggttacaaataaagcaatagcatcacaaatttcacaaataaagcatttttttcactgcattctagttgtggtttgtccaaactcatcaatgtatcttatcatgtctggatcgatccgaaccccttcctcgaccaattctcatgtttgacagcttatcatcgcagatccgggcaacgttgttgcattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccaggatggagatctcatcaggcttgctgttcctgggccacctaatagtgctcacctatggccaccccaccctaaaaacacctgagagtgtgacagggacctggaaaggagatgtgaagattcagtgcatctatgatcccctgagaggctacaggcaagttttggtgaaatggctggtaagacacggctctgactccgtcaccatcttcctacgtgactccactggagaccatatccagcaggcaaagtacagaggccgcctgaaagtgagccacaaagttccaggagatgtgtccctccaaataaataccctgcagatggatgacaggaatcactatacatgtgaggtcacctggcagactcctgatggaaaccaagtaataagagataagatcattgagctccgtgttcggaaatataatccacctagaatcaatactgaagcacctacaaccctgcactcctctttggaagcaacaactataatgagttcaacctctgacttgaccactaatgggactggaaaacttgaggagaccattgctggttcagggaggaacctgctagcgcagccgcagccgaaaccgcagccgcagccgcagccgcagccgaaaccgcagccgaaaccggaaccggaagctttgggagactgctgcccacagatgcttcgagaactccaggagactaatgcggcgctgcaagacgtgagagagctcttgcgacagcaggtcaaggagatcaccttcctgaagaatacggtgatggaatgtgacgcttgcggaggatctggtctagacgactacaaggatgacgacgacaagtaggggcccgaacaaaaactcatctcagaagaggatctgaatagcgccgtcgaccatcatcatcatcatcattgagtttaaacgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtggggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaggtcgactctaga

SEQ ID NO: 25 - DNA, 19 bp, Artificial Sequence
  LV43-XM087714f primer
tgctgacgagagatggtgg

SEQ ID NO: 26 - DNA, 20 bp, Artificial Sequence
  LV44-XM087714b primer
ccacagcctttagatgacgg

SEQ ID NO: 27 - DNA, 27 bp, Artificial Sequence
  LV49-XM087714f primer
gggggtacctgctgacgagagatggtg

SEQ ID NO: 28 - DNA, 25 bp, Artificial Sequence
  LV48-XM087714b primer
cggctagcccgggtacgaacacgtc

SEQ ID NO: 29 - DNA, 20 bp, Artificial Sequence
  LV50-XP087460f primer
tttccatctgaggcaagaag

SEQ ID NO: 30 - DNA, 22 bp, Artificial Sequence
  LV60-hsB7-H5b primer
ttcctcatgtcctataccaagg

SEQ ID NO: 31 - DNA, 30 bp, Artificial Sequence
  LV56-sec-hsB7-H5f primer
ggggtaccatgtctctggtggaacttttgc

SEQ ID NO: 32 - DNA, 26 bp, Artificial Sequence
  LV57-sec-hsB7-H5b primer
cggctagcccaatgttcctgggctgg

SEQ ID NO: 33 - DNA, 17 bp, Artificial Sequence
  JS7-mB7-H5f primer
atgactcggcggcgctc

SEQ ID NO: 34 - DNA, 23 bp, Artificial Sequence
  JS8-mB7-H5r primer
ctataccagggaccctgctcgac

SEQ ID NO: 35 - DNA, 26 bp, Artificial Sequence
  MSt-1mB7-H5for primer
ggggtaccatgactcggcggcgctcc

SEQ ID NO: 36 - DNA, 28 bp, Artificial Sequence
  MSt-2mB7-H5rev primer
gggctagcacgggtgagataacctggag

SEQ ID NO: 37 - DNA, 21 bp, Artificial Sequence
  LV80-mC18f
gtagcttcaaataggatggag

SEQ ID NO: 38 - DNA, 20 bp, Artificial Sequence
  LV81-mC18b
aaactgtgttcagcaggcag

SEQ ID NO: 39 - DNA, 26 bp, Artificial Sequence
  LV82-mC18f primer
gggtaccaggatggagatctcatcag

SEQ ID NO: 40 - DNA, 23 bp, Artificial Sequence
  LV83-mC18b primer
ggctagcaggttcctccctgaac

SEQ ID NO: 41 - DNA, 1210 bp, homo sapiens
  CDS (6)..(1202)
ctgtgatggggatcttactgggcctgctactcctggggcacctaacagtggacacttatggccgtcccatcctggaagtgccagagagtgtaacaggaccttggaaaggggatgtgaatcttccctgcacctatgaccccctgcaaggctacacccaagtcttggtgaagtggctggtacaacgtggctcagaccctgtcaccatctttctacgtgactcttctggagaccatatccagcaggcaaagtaccagggccgcctgcatgtgagccacaaggttccaggagatgtatccctccaattgagcaccctggagatggatgaccggagccactacacgtgtgaagtcacctggcagactcctgatggcaaccaagtcgtgagagataagattactgagctccgtgtccagaaactctctgtctccaagcccacagtgacaactggcagcggttatggcttcacggtgccccagggaatgaggattagccttcaatgccaggctcggggttctcctcccatcagttatatttggtataagcaacagactaataaccaggaacccatcaaagtagcaaccctaagtaccttactcttcaagcctgcggtgatagccgactcaggctcctatttctgcactgccaagggccaggttggctctgagcagcacagcgacattgtgaagtttgtggtcaaagactcctcaaagctactcaagaccaagactgaggcacctacaaccatgacataccccttgaaagcaacatctacagtgaagcagtcctgggactggaccactgacatggatggctaccttggagagaccagtgctgggccaggaaagagcctgcctgtctttgccatcatcctcatcatctccttgtgctgtatggtggtttttaccatggcctatatcatgctctgtcggaagacatcccaacaagagcatgtctacgaagcagccagggcacatgccagagaggccaacgactctggagaaaccatgagggtggccatcttcgcaagtggctgctccagtgatgagccaacttcccagaatctgggcaacaactactctgatgagccctgcataggacaggagtaccagatcatcgcccagatcaatggcaactacgcccgcctgctggacacagttcctctggattatgagtttctggccactgagggcaaaagtgtctgttaaaaatg

SEQ ID NO: 42 - protein, 399 aa, homo sapiens
MGILLGLLLLGHLTVDTYGRPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQKLSVSKPTVTTGSGYGFTVPQGMRISLQCQARGSPPISYIWYKQQTNNQEPIKVATLSTLLFKPAVIADSGSYFCTAKGQVGSEQHSDIVKFVVKDSSKLLKTKTEAPTTMTYPLKATSTVKQSWDWTTDMDGYLGETSAGPGKSLPVFAIILIISLCCMVVFTMAYIMLCRKTSQQEHVYEAARAHAREANDSGETMRVAIFASGCSSDEPTSQNLGNNYSDEPCIGQEYQIIAQINGNYARLLDTVPLDYEFLATEGKSVC

SEQ ID NO: 43 - DNA, 844 bp, homo sapiens
  CDS (1)..(843)
  misc_feature (513)..(513): T at position 513 might be a C (silent mutation)
atggggatcttactgggcctgctactcctggggcacctaacagtggacacttatggccgtcccatcctggaagtgccagagagtgtaacaggaccttggaaaggggatgtgaatcttccctgcacctatgaccccctgcaaggctacacccaagtcttggtgaagtggctggtacaacgtggctcagaccctgtcaccatctttctacgtgactcttctggagaccatatccagcaggcaaagtaccagggccgcctgcatgtgagccacaaggttccaggagatgtatccctccaattgagcaccctggagatggatgaccggagccactacacgtgtgaagtcacctggcagactcctgatggcaaccaagtcgtgagagataagattactgagctccgtgtccagaaactctctgtctccaagcccacagtgacaactggcagcggttatggcttcacggtgccccagggaatgaggattagccttcaatgccaggctcggggttctcctcccatcagttatatttggtataagcaacagactaataaccaggaacccatcaaagtagcaaccctaagtaccttactcttcaagcctgcggtgatagccgactcaggctcctatttctgcactgccaagggccaggttggctctgagcagcacagcgacattgtgaagtttgtggtcaaagactcctcaaagctactcaagaccaagactgaggcacctacaaccatgacataccccttgaaagcaacatctacagtgaagcagtcctgggactggaccactgacatggatggctaccttggagagaccagtgctgggccaggaaagctag

SEQ ID NO: 44 - protein, 281 aa, homo sapiens
MGILLGLLLLGHLTVDTYGRPILEVPESVTGPWKGDVNLPCTYDPLQGYTQVLVKWLVQRGSDPVTIFLRDSSGDHIQQAKYQGRLHVSHKVPGDVSLQLSTLEMDDRSHYTCEVTWQTPDGNQVVRDKITELRVQKLSVSKPTVTTGSGYGFTVPQGMRISLQCQARGSPPISYIWYKQQTNNQEPIKVATLSTLLFKPAVIADSGSYFCTAKGQVGSEQHSDIVKFVVKDSSKLLKTKTEAPTTMTYPLKATSTVKQSWDWTTDMDGYLGETSAGPGKL

SEQ ID NO: 45 - DNA, 10615 bp, Artificial Sequence
  pCEP-hsB7-H6-COMP-FLAG
gcattctagttgtggtttgtccaaactcatcaatgtatcttatcatgtctggatcgatccgaaccccttcctcgaccaattctcatgtttgacagcttatcatcgcagatccgggcaacgttgttgcattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccgccaccatggggatcttactgggcctgctactcctggggcacctaacagtggacacttatggccgtcccatcctggaagtgccagagagtgtaacaggaccttggaaaggggatgtgaatcttccctgcacctatgaccccctgcaaggctacacccaagtcttggtgaagtggctggtacaacgtggctcagaccctgtcaccatctttctacgtgactcttctggagaccatatccagcaggcaaagtaccagggccgcctgcatgtgagccacaaggttccaggagatgtatccctccaattgagcaccctggagatggatgaccggagccactacacgtgtgaagtcacctggcagactcctgatggcaaccaagtcgtgagagataagattactgagctccgtgtccagaaactctctgtctccaagcccacagtgacaactggcagcggttatggcttcacggtgccccagggaatgaggattagccttcaatgccaggctcggggttctcctcccatcagttatatttggtataagcaacagactaataaccaggaacccatcaaagtagcaaccctaagtaccttactcttcaagcctgcggtgatagccgactcaggctcctatttctgcactgccaagggccaggttggctctgagcagcacagcgacattgtgaagtttgtggtcaaagactcctcaaagctactcaagaccaagactgaggcacctacaaccatgacataccccttgaaagcaacatctacagtgaagcagtcctgggactggaccactgacatggatggctaccttggagagaccagtgctgggccaggaaagctagcgcagccgcagccgaaaccgcagccgcagccgcagccgcagccgaaaccgcagccgaaaccggaaccggaagctttgggagactgctgcccacagatgcttcgagaactccaggagactaatgcggcgctgcaagacgtgagagagctcttgcgacagcaggtcaaggagatcaccttcctgaagaatacggtgatggaatgtgacgcttgcggaggatctggtctagacgactacaaggatgacgacgacaagtaggggcccgaacaaaaactcatctcagaagaggatctgaatagcgccgtcgaccatcatcatcatcatcattgagtttaaacgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtggggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaggtcgactctagaggatcgatccccgccgccggacgaactaaacctgactacggcatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcgccgcgtgcggctgctggagatggcggacgcgatggatatgttctgccaagggttggtttgcgcattcacagttctccgcaagaattgattggctccaattcttggagtggtgaatccgttagcgaggccatccagcctcgcgtcgaactagatgatccgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggagggtgaccgccacgaggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgaccccgaccgccacatcgaacgcgtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgacccggtccgacggcggcccacgggtcccaggggggtcgacctcgaaacttgtttattgcagcttataatggttacaaataaagcaatagcatcacaaatttcacaaataaagcatttttttcact

SEQ ID NO: 46 - DNA, 11059 bp, Artificial Sequence
  pCEP-hsB7-H6-Xa1-Fc*
gcattctagttgtggtttgtccaaactcatcaatgtatcttatcatgtctggatcgatccgaaccccttcctcgaccaattctcatgtttgacagcttatcatcgcagatccgggcaacgttgttgcattgctgcaggcgcagaactggtaggtatggaagatctatacattgaatcaatattggcaattagccatattagtcattggttatatagcataaatcaatattggctattggccattgcatacgttgtatctatatcataatatgtacatttatattggctcatgtccaatatgaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtccgccccctattgacgtcaatgacggtaaatggcccgcctggcattatgcccagtacatgaccttacgggactttcctacttggcagtacatctacgtattagtcatcgctattaccatggtgatgcggttttggcagtacaccaatgggcgtggatagcggtttgactcacggggatttccaagtctccaccccattgacgtcaatgggagtttgttttggcaccaaaatcaacgggactttccaaaatgtcgtaataaccccgccccgttgacgcaaatgggcggtaggcgtgtacggtgggaggtctatataagcagagctcgtttagtgaaccgtcagatctctagaagctgggtaccgccaccatggggatcttactgggcctgctactcctggggcacctaacagtggacacttatggccgtcccatcctggaagtgccagagagtgtaacaggaccttggaaaggggatgtgaatcttccctgcacctatgaccccctgcaaggctacacccaagtcttggtgaagtggctggtacaacgtggctcagaccctgtcaccatctttctacgtgactcttctggagaccatatccagcaggcaaagtaccagggccgcctgcatgtgagccacaaggttccaggagatgtatccctccaattgagcaccctggagatggatgaccggagccactacacgtgtgaagtcacctggcagactcctgatggcaaccaagtcgtgagagataagattactgagctccgtgtccagaaactctctgtctccaagcccacagtgacaactggcagcggttatggcttcacggtgccccagggaatgaggattagccttcaatgccaggctcggggttctcctcccatcagttatatttggtataagcaacagactaataaccaggaacccatcaaagtagcaaccctaagtaccttactcttcaagcctgcggtgatagccgactcaggctcctatttctgcactgccaagggccaggttggctctgagcagcacagcgacattgtgaagtttgtggtcaaagactcctcaaagctactcaagaccaagactgaggcacctacaaccatgacataccccttgaaagcaacatctacagtgaagcagtcctgggactggaccactgacatggatggctaccttggagagaccagtgctgggccaggaaagctagcgatcgaaggtcgcaagcttactcacacatgcccaccgtgcccagcacctgaagccgagggggcaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgtgtggtcagcgtcctcaccgtcctgcaccaggactggctgaatggcaaggagtacaagtgcaaggtctccaacaaagccctcccagcctccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgttggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaatgactcgaggcccgaacaaaaactcatctcagaagaggatctgaatagcgccgtcgaccatcatcatcatcatcattgagtttaacgatccagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtggggaggttttttaaagcaagtaaaacctctacaaatgtggtatggctgattatgatccggctgcctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggcgcagccatgaggtcgactctagaggatcgatccccgccgccggacgaactaaacctgactacggcatctctgccccttcttcgcggggcagtgcatgtaatcccttcagttggttggtacaacttgccaactgggccctgttccacatgtgacacggggggggaccaaacacaaaggggttctctgactgtagttgacatccttataaatggatgtgcacatttgccaacactgagtggctttcatcctggagcagactttgcagtctgtggactgcaacacaacattgcctttatgtgtaactcttggctgaagctcttacaccaatgctgggggacatgtacctcccaggggcccaggaagactacgggaggctacaccaacgtcaatcagaggggcctgtgtagctaccgataagcggaccctcaagagggcattagcaatagtgtttataaggcccccttgttaaccctaaacgggtagcatatgcttcccgggtagtagtatatactatccagactaaccctaattcaatagcatatgttacccaacgggaagcatatgctatcgaattagggttagtaaaagggtcctaaggaacagcgatatctcccaccccatgagctgtcacggttttatttacatggggtcaggattccacgagggtagtgaaccattttagtcacaagggcagtggctgaagatcaaggagcgggcagtgaactctcctgaatcttcgcctgcttcttcattctccttcgtttagctaatagaataactgctgagttgtgaacagtaaggtgtatgtgaggtgctcgaaaacaaggtttcaggtgacgcccccagaataaaatttggacggggggttcagtggtggcattgtgctatgacaccaatataaccctcacaaaccccttgggcaataaatactagtgtaggaatgaaacattctgaatatctttaacaatagaaatccatggggtggggacaagccgtaaagactggatgtccatctcacacgaatttatggctatgggcaacacataatcctagtgcaatatgatactggggttattaagatgtgtcccaggcagggaccaagacaggtgaaccatgttgttacactctatttgtaacaaggggaaagagagtggacgccgacagcagcggactccactggttgtctctaacacccccgaaaattaaacggggctccacgccaatggggcccataaacaaagacaagtggccactcttttttttgaaattgtggagtgggggcacgcgtcagcccccacacgccgccctgcggttttggactgtaaaataagggtgtaataacttggctgattgtaaccccgctaaccactgcggtcaaaccacttgcccacaaaaccactaatggcaccccggggaatacctgcataagtaggtgggcgggccaagataggggcgcgattgctgcgatctggaggacaaattacacacacttgcgcctgagcgccaagcacagggttgttggtcctcatattcacgaggtcgctgagagcacggtgggctaatgttgccatgggtagcatatactacccaaatatctggatagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctaatagagattagggtagtatatgctatcctaatttatatctgggtagcatatactacccaaatatctggatagcatatgctatcctaatctatatctgggtagcatatgctatcctaatctatatctgggtagcataggctatcctaatctatatctgggtagcatatgctatcctaatctatatctgggtagtatatgctatcctaatttatatctgggtagcataggctatcctaatctatatctgggtagcatatgctatcctaatctatatctgggtagtatatgctatcctaatctgtatccgggtagcatatgctatcctcatgcatatacagtcagcatatgatacccagtagtagagtgggagtgctatcctttgcatatgccgccacctcccaagggggcgtgaattttcgctgcttgtccttttcctgcatgctggttgctcccattcttaggtgaatttaaggaggccaggctaaagccgtcgcatgtctgattgctcaccaggtaaatgtcgctaatgttttccaacgcgagaaggtgttgagcgcggagctgagtgacgtgacaacatgggtatgcccaattgccccatgttgggaggacgaaaatggtgacaagacagatggccagaaatacaccaacagcacgcatgatgtctactggggatttattctttagtgcgggggaatacacggcttttaatacgattgagggcgtctcctaacaagttacatcactcctgcccttcctcaccctcatctccatcacctccttcatctccgtcatctccgtcatcaccctccgcggcagccccttccaccataggtggaaaccagggaggcaaatctactccatcgtcaaagctgcacacagtcaccctgatattgcaggtaggagcgggctttgtcataacaaggtccttaatcgcatccttcaaaacctcagcaaatatatgagtttgtaaaaagaccatgaaataacagacaatggactcccttagcgggccaggttgtgggccgggtccaggggccattccaaaggggagacgactcaatggtgtaagacgacattgtggaatagcaagggcagttcctcgccttaggttgtaaagggaggtcttactacctccatatacgaacacaccggcgacccaagttccttcgtcggtagtcctttctacgtgactcctagccaggagagctcttaaaccttctgcaatgttctcaaatttcgggttggaacctccttgaccacgatgctttccaaaccaccctccttttttgcgcctgcctccatcaccctgaccccggggtccagtgcttgggccttctcctgggtcatctgcggggccctgctctatcgctcccgggggcacgtcaggctcaccatctgggccaccttcttggtggtattcaaaataatcggcttcccctacagggtggaaaaatggccttctacctggagggggcctgcgcggtggagacccggatgatgatgactgactactgggactcctgggcctcttttctccacgtccacgacctctccccctggctctttcacgacttccccccctggctctttcacgtcctctaccccggcggcctccactacctcctcgaccccggcctccactacctcctcgaccccggcctccactgcctcctcgaccccggcctccacctcctgctcctgcccctcctgctcctgcccctcctcctgctcctgcccctcctgcccctcctgctcctgcccctcctgcccctcctgctcctgcccctcctgcccctcctgctcctgcccctcctgcccctcctcctgctcctgcccctcctgcccctcctcctgctcctgcccctcctgcccctcctgctcctgcccctcctgcccctcctgctcctgcccctcctgcccctcctgctcctgcccctcctgctcctgcccctcctgctcctgcccctcctgctcctgcccctcctgcccctcctgcccctcctcctgctcctgcccctcctgctcctgcccctcctgcccctcctgcccctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctgcccctcctcctgctcctgcccctcctcctgctcctgcccctcctgcccctcctgcccctcctcctgctcctgcccctcctcctgctcctgcccctcctgcccctcctgcccctcctgcccctcctcctgctcctgcccctcctcctgctcctgcccctcctgctcctgcccctcccgctcctgctcctgctcctgttccaccgtgggtccctttgcagccaatgcaacttggacgtttttggggtctccggacaccatctctatgtcttggccctgatcctgagccgcccggggctcctggtcttccgcctcctcgtcctcgtcctcttccccgtcctcgtccatggttatcaccccctcttctttgaggtccactgccgccggagccttctggtccagatgtgtctcccttctctcctaggccatttccaggtcctgtacctggcccctcgtcagacatgattcacactaaaagagatcaatagacatctttattagacgacgctcagtgaatacagggagtgcagactcctgccccctccaacagcccccccaccctcatccccttcatggtcgctgtcagacagatccaggtctgaaaattccccatcctccgaaccatcctcgtcctcatcaccaattactcgcagcccggaaaactcccgctgaacatcctcaagatttgcgtcctgagcctcaagccaggcctcaaattcctcgtccccctttttgctggacggtagggatggggattctcgggacccctcctcttcctcttcaaggtcaccagacagagatgctactggggcaacggaagaaaagctgggtgcggcctgtgaggatcagcttatcgatgataagctgtcaaacatgagaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtgttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctgcgccgcgtgcggctgctggagatggcggacgcgatggatatgttctgccaagggttggtttgcgcattcacagttctccgcaagaattgattggctccaattcttggagtggtgaatccgttagcgaggccatccagcctcgcgtcgaactagatgatccgctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggagggtgaccgccacgaggtgccgccaccatcccctgacccacgcccctgacccctcacaaggagacgaccttccatgaccgagtacaagcccacggtgcgcctcgccacccgcgacgacgtcccccgggccgtacgcaccctcgccgccgcgttcgccgactaccccgccacgcgccacaccgtcgaccccgaccgccacatcgaacgcgtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgagtgcccgaaggaccgcgcgacctggtgcatgacccgcaagcccggtgcctgacgcccgccccacgacccgcagcgcccgaccgaaaggagcgcacgacccggtccgacggcggcccacgggtcccaggggggtcgacctcgaaacttgtttattgcagcttataatggttacaaataaagcaatagcatcacaaatttcacaaataaagcatttttttcact

SEQ ID NO: 47 - DNA, 20 bp, Artificial Sequence
  B76-1 oligonucleotide
aggaggctggaagaaaggac

SEQ ID NO: 48 - DNA, 18 bp, Artificial Sequence
  B76-2 oligonucleotide
cccccggcagagatacta

SEQ ID NO: 49 - DNA, 32 bp, Artificial Sequence
  B76-3 oligonucleotide
ggtaccgccaccatggggatcttactgggcct

SEQ ID NO: 50 - DNA, 23 bp, Artificial Sequence
  B76-4 oligonucleotide
gctagctttcctggcccagcact